(19) United States
(12) Patent Application Publication
(10) Pub. No.: US 2021/0330807 A1
(43) Pub. Date: Oct. 28, 2021

(54) NOVEL HYDROGEL CONJUGATES

(71) Applicant: Ascendis Pharma A/S, Hellerup (DK)

(72) Inventors: Sebastian STARK, Heidelberg (DE); Tobias VOIGT, Heidelberg (DE); Nicola BISEK, Heidelberg (DE)

(21) Appl. No.: 17/280,738

(22) PCT Filed: Sep. 25, 2019
(86) PCT No.: PCT/EP2019/075881

§ 371 Date: Mar. 26, 2021

(30) Foreign Application Priority Data
Sep. 26, 2018 (EP) 18196864.5
Jan. 4, 2019 (EP) 19150396.0
Jun. 21, 2019 (EP) 19181814.5

(51) Int. Cl. A61K 47/69; A61K 47/60; A61K 47/54; A61K 31/665; A61K 31/42; A61K 38/12; A61K 38/15; A61K 31/65; A61K 31/395; A61K 31/43; A61K 31/545; A61K 31/4166; A61K 31/409; A61K 31/7056

(52) U.S. Cl. CPC A61K 47/6903; A61K 47/545; A61K 47/542; A61K 31/665; A61K 31/42; A61K 38/12; A61K 38/15; A61K 31/65; A61K 31/395; A61K 31/43; A61K 31/545; A61K 31/4166; A61K 31/409; A61K 31/7056; A61K 47/60

(57) ABSTRACT

The present invention relates to conjugates comprising backbone moieties that are crosslinked via particular crosslinker moieties to which a plurality of drug moieties are covalently and reversibly conjugated. It also relates to their use as medicaments and their use in the diagnosis, prevention and treatment of diseases.

The present invention relates to conjugates comprising backbone moieties that are crosslinked via particular crosslinker moieties to which a plurality of drug moieties are covalently and reversibly conjugated. It also relates to their use as medicaments and their use in the diagnosis, prevention and treatment of diseases.

Hydrogels are three-dimensional, hydrophilic or amphiphilic polymeric networks capable of taking up large quantities of water. These networks may be composed of various polymers and are insoluble due to the presence of covalent chemical and/or physical crosslinks.

Hydrogels can be used for many applications, such as for the sustained release of drug molecules. Such drug molecules may either be non-covalently embedded or covalently and reversibly attached to the hydrogel. When hydrogels are used for covalent attachment of drugs, they may need to have specific characteristics, such as a particular drug loading capacity or a certain degradation profile. Examples for such hydrogels are shown in WO2011/012715A1 and WO2014/056926A1. However, there is always a need for hydrogels with different features.

Thus, it is an object of the present invention to provide additional novel hydrogels having useful and surprising characteristics.

This object is achieved with a conjugate comprising a water-insoluble hydrogel Z, wherein said conjugate comprises a plurality of moieties -L²-L¹-D covalently conjugated to Z,
wherein
each -D is drug moiety;
each -L¹- is independently a linker moiety to which -D is covalently and reversibly conjugated;
each -L²- is independently either a chemical bond or a spacer moiety;
Z is a PEG-based hydrogel comprising a plurality of backbone moieties that are crosslinked via crosslinker moieties —CL-, either directly or via a spacer moiety —SP— between a backbone moiety and —CL-, and wherein —CL- is of formula (A)

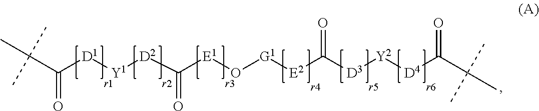

wherein
dashed lines indicate attachment to a backbone moiety or to a spacer moiety —SP—;
—Y¹— is of formula

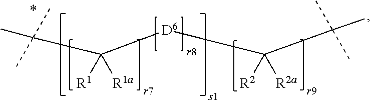

wherein the dashed line marked with the asterisk indicates attachment to -D¹- and the unmarked dashed line indicates attachment to -D²-;
—Y²— is of formula

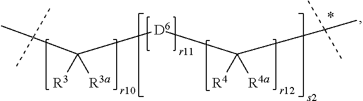

wherein the dashed line marked with the asterisk indicates attachment to -D⁴- and the unmarked dashed line indicates attachment to -D³-;
-E¹- is of formula

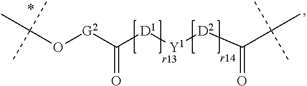

wherein the dashed line marked with the asterisk indicates attachment to —(C═O)— and the unmarked dashed line indicates attachment to —O—;
-E²- is of formula

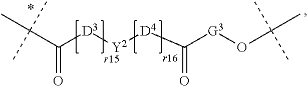

wherein the dashed line marked with the asterisk indicates attachment to -G¹- and the unmarked dashed line indicates attachment to —(C═O)—;
-G¹- is of formula

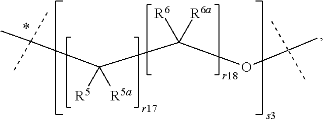

wherein the dashed line marked with the asterisk indicates attachment to —O— and the unmarked dashed line indicates attachment to -E²-;
-G²- is of formula

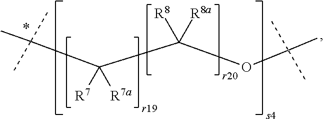

wherein the dashed line marked with the asterisk indicates attachment to —O— and the unmarked dashed line indicates attachment to —(C═O)—;
-G³- is of formula

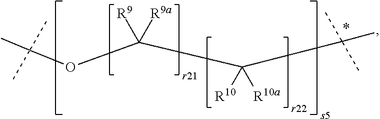

wherein the dashed line marked with the asterisk indicates attachment to —O— and the unmarked dashed line indicates attachment to —(C═O)—;
-D¹-, -D²-, -D³-, -D⁴-, -D⁵- and -D⁶- are identical or different and each is independently of the others selected from the group comprising —O—, —NR¹¹—, —N⁺R¹²R^{12a}, —, —S—, —(S═O)—, —(S(O)₂)—, —C(O)—, —P(O)R¹³—, —P(O)(OR¹³) and —CR¹⁴R^{14a}—;
—R¹, —R^{1a}, —R², —R^{2a}, —R³, —R^{3a}, —R⁴, —R^{4a}, —R⁵, —R^{5a}, —R⁶, —R^{6a}, —R⁷, —R^{7a}, —R⁸, —R^{8a}, —R⁹, —R^{9a}, —R¹⁰, —R^{10a}, —R¹¹, —R¹², —R^{12a}, —R¹³, —R¹⁴ and —R^{14a} are identical or different and each is independently of the others selected from the group consisting of —H and C₁₋₆ alkyl;

optionally, one or more of the pairs —R¹/—R^{1a}, —R²/—R^{2a}, —R³/—R^{3a}, —R⁴/—R^{4a}, —R¹/—R², —R³/—R⁴, —R^{1a}/—R^{2a}, —R^{3a}/—R^{4a}, —R¹²/—R^{12a}, and —R¹⁴/—R^{14a} form a chemical bond or are joined together with the atom to which they are attached to form a C₃₋₈ cycloalkyl or to form a ring A or are joined together with the atom to which they are attached to form a 4- to 7-membered heterocyclyl or 8- to 11-membered heterobicyclyl or adamantyl; A is selected from the group consisting of phenyl, naphthyl, indenyl, indanyl and tetralinyl;
r1, r2, r5, r6, r13, r14, r15 and r16 are independently 0 or 1;
r3, r4 are independently 0, 1, 2, 3, or 4, with the provision that r3+r4≥1;
r7, r8, r9, r10, r11, r12 are independently 0, 1, 2, 3, or 4;
r17, r18, r19, r20, r21 and r22 are independently 1, 2, 3, 4, 5, 6, 7, 8, 9 or 10;
s1, s2, s4, s5 are independently 1, 2, 3, 4, 5 or 6; and
s3 ranges from 1 to 900.

It was surprisingly found that such hydrogels provide not only a high degree of drug loading, but also beneficial degradation kinetics.

Within the present invention the terms are used having the meaning as follows.

As used herein the term “spacer” refers to a moiety that connects at least two other moieties with each other.

As used herein the term “crosslinker” refers to a moiety that is a connection between two backbone moieties, either directly or via a spacer moiety.

As used herein, the term “water-insoluble” refers to a compound of which less than 1 g can be dissolved in one liter of water at 20° C. to form a homogeneous solution. Accordingly, the term “water-soluble” refers to a compound of which 1 g or more can be dissolved in one liter of water at 20° C. to form a homogeneous solution.

As used herein, the term “a π-electron-pair-donating heteroaromatic N-comprising moiety” refers to the moiety which after cleavage of the linkage between -D and -L¹- results in a drug D-H and wherein the drug moiety -D and analogously the corresponding D-H comprises at least one, such as one, two, three, four, five, six, seven, eight, nine or ten heteroaromatic nitrogen atoms that donate a π-electron pair to the aromatic π-system. Examples of chemical structures comprising such heteroaromatic nitrogens that donate a π-electron pair to the aromatic π-system include, but are not limited to, pyrrole, pyrazole, imidazole, isoindazole, indole, indazole, purine, tetrazole, triazole and carbazole. For example, in the imidazole ring below the heteroaromatic nitrogen which donates a π-electron pair to the aromatic π-system is marked with “#”:

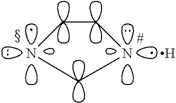

The π-electron-pair-donating heteroaromatic nitrogen atoms do not comprise heteroaromatic nitrogen atoms which only donate one electron (i.e. not a pair of π-electrons) to the aromatic π-system, such as for example the nitrogen that is marked with “§” in the abovementioned imidazole ring structure. The drug D-H may exist in one or more tautomeric forms, such as with one hydrogen atom moving between at least two heteroaromatic nitrogen atoms. In all such cases, the linker moiety is covalently and reversibly attached at a heteroaromatic nitrogen that donates a π-electron pair to the aromatic π-system.

As used herein, the term “drug” refers to a substance used in the treatment, cure, prevention or diagnosis of a disease or used to otherwise enhance physical or mental well-being of a patient. If a drug is conjugated to another moiety, the moiety of the resulting product that originated from the drug is referred to as “drug moiety”.

It is understood that the conjugates of the present invention are prodrugs.

As used herein the term “prodrug” refers to a biologically active moiety reversibly and covalently connected to a specialized protective group through a reversible prodrug linker moiety which is a linker moiety comprising a reversible linkage with the biologically active moiety and wherein the specialized protective group alters or eliminates undesirable properties in the parent molecule. This also includes the enhancement of desirable properties in the drug and the suppression of undesirable properties. The specialized non-toxic protective group may also be referred to as “carrier”. A prodrug releases the reversibly and covalently bound biologically active moiety in the form of its corresponding drug. In other words, a prodrug is a conjugate comprising a drug moiety, which is covalently and reversibly conjugated to a carrier moiety via a reversible prodrug linker moiety, which covalent and reversible conjugation of the carrier to the reversible prodrug linker moiety is either directly or through a spacer. Such conjugate preferably releases the formerly conjugated drug moiety in the form of a free drug, in which case the reversible linker or reversible prodrug linker is a traceless linker. The conjugates of the present invention are prodrugs.

As used herein, the term “sustained release” refers to the property of a compound, such as the conjugates of the present invention, to release a drug, such as one or more antibiotic but also any other class of drug, with a release half-life of at least 1 day.

As used herein, the term “free form” of a drug means the drug in its unmodified, pharmacologically active form.

As used herein, the term “reversible”, “reversibly”, “degradable” or “degradably” refers to a bond that is cleavable under physiological conditions, which are aqueous buffer at pH 7.4 and 37° C., with a half-life ranging from one day to three months, preferably from two days to two months, even more preferably from two days to one month. Cleavage is preferably non-enzymatically. Accordingly, the term “stable” with regard to the attachment of a first moiety to a second moiety means that the linkage that connects said first and second moiety exhibits a half-life of more than three months under physiological conditions.

As used herein, the term “reagent” means a chemical compound, which comprises at least one functional group for reaction with the functional group of another chemical compound or drug. It is understood that a drug comprising a functional group is also a reagent.

As used herein, the term “moiety” means a part of a molecule, which lacks one or more atoms compared to the corresponding reagent. If, for example, a reagent of the formula “H—X—H” reacts with another reagent and becomes part of the reaction product, the corresponding moiety of the reaction product has the structure “H—X—” or “—X—”, whereas each indicates attachment to another moiety. Accordingly, a drug moiety is released from a reversible linkage as a drug.

It is understood that if the chemical structure of a group of atoms is provided which group of atoms is attached to two moieties or is interrupting a moiety, said sequence or chemical structure can be attached to the two moieties in either orientation, unless explicitly stated otherwise. For example, a moiety “—C(O)N(R¹)—” can be attached to two moieties or interrupting a moiety either as “—C(O)N(R¹)—” or as “—N(R¹)C(O)—”. Similarly, a moiety

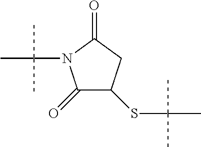

can be attached to two moieties or can interrupt a moiety either as

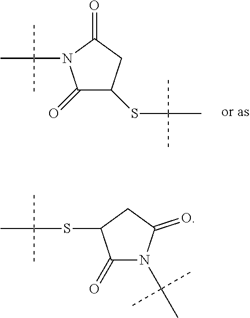

The term “substituted” as used herein means that one or more —H atom(s) of a molecule or moiety are replaced by a different atom or a group of atoms, which are referred to as “substituent”.

As used herein, the term “substituent” refers in certain embodiments to a moiety selected from the group consisting of halogen, —CN, —COOR^{x1}, —OR^{x1}, —C(O)R^{x1}, —C(O)N(R^{x1}R^{x1a}), —S(O)₂N(R^{x1}R^{x1a}), —S(O)N(R^{x1}R^{x1a}), —S(O)₂R^{x1}, —S(O)R^{x1}, —N(R^{x1})S(O)₂N(R^{x1a}R^{x1b}), —SR^{x1}, —N(R^{x1}R^{x1a}), —NO₂, —OC(O)R^{x1}, —N(R^{x1})C(O)R^{x1a}, —N(R^{x1})S(O)₂R^{x1a}, —N(R^{x1})S(O)R^{x1a}, —N(R^{x1})C(O)OR^{x1a}, —N(R^{x1})C(O)N(R^{x1a}R^{x1b}), —OC(O)N(R^{x1}R^{x1a}), -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—, —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—;
—R^{x1}, —R^{x1a}, —R^{x1b} are independently of each other selected from the group consisting of —H, -T⁰, C₁₋₅₀ alkyl, C₂₋₅₀ alkenyl, and C₂₋₅₀ alkynyl; wherein -T⁰, C₁₋₅₀ alkyl, C₂₋₅₀ alkenyl, and C₂₋₅₀ alkynyl are optionally substituted with one or more —R^{x2}, which are the same or different and wherein C₁₋₅₀ alkyl, C₂₋₅₀ alkenyl, and C₂₋₅₀ alkynyl are optionally interrupted by one or more groups selected from the group consisting of -T⁰-, —C(O)O—, —O—, —C(O)—, —C(O)N(R^{x3})—, —S(O)₂N(R^{x3})—, —S(O)N(R^{x3})—; —S(O)₂—, —S(O)—, —N(R^{x3})S(O)₂N(R^{x3a})—, —S—, —N(R^{x3})—, —OC(OR^{x3})(R^{x3a})—, —N(R^{x3})C(O)N(R^{x3a})—, and —OC(O)N(R^{x3})—; each T° is independently selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T° is independently optionally substituted with one or more —R^{x2}, which are the same or different;
each —R^{x2} is independently selected from the group consisting of halogen, —CN, oxo (═O), —COOR^{x4}, —OR^{x4}, —C(O)R^{x4}, —C(O)N(R^{x4}R^{x4a}), —S(O)₂N(R^{x4}R^{x4a}), —S(O)N(R^{x4}R^{x4a}), —S(O)₂R^{x4}, —S(O)R^{x4}, —N(R^{x4})S(O)₂N(R^{x4a}R^{x4b}), —SR^{x4}, —N(R^{x4}R^{x4a}), —NO₂, —OC(O)R^{x4}, —N(R^{x4})C(O)R^{x4a}, —N(R^{x4})S(O)₂R^{x4a}, —N(R^{x4})S(O)R^{x4a}, —N(R^{x4})C(O)OR^{x4a}, —N(R^{X4})C(O)N(R^{x4a}R^{x4b}), —OC(O)N(R^{x4}R^{x4a}), and C₁₋₆ alkyl; wherein C₁₋₆ alkyl is optionally substituted with one or more halogen, which are the same or different;
each —R^{x3}, —R^{x3a}, —R^{x4}, —R^{x4a}, —R^{x4b} is independently selected from the group consisting of —H and C₁₋₆ alkyl; wherein C₁₋₆ alkyl is optionally substituted with one or more halogen, which are the same or different.

In certain embodiments a maximum of 6 —H atoms of an optionally substituted molecule are independently replaced by a substituent, e.g. 5 —H atoms are independently replaced by a substituent, 4 —H atoms are independently replaced by a substituent, 3 —H atoms are independently replaced by a substituent, 2 —H atoms are independently replaced by a substituent, or 1 —H atom is replaced by a substituent.

As used herein, the term “hydrogel” means a hydrophilic or amphiphilic polymeric network composed of homopolymers or copolymers, which is insoluble due to the presence of hydrophobic interactions, hydrogen bonds, ionic interactions and/or covalent chemical crosslinks. The crosslinks provide the network structure and physical integrity.

As used herein the term “about” in combination with a numerical value is used to indicate a range ranging from and including the numerical value plus and minus no more than 25% of said numerical value, more preferably no more than 20% of said numerical value and most preferably no more than 10% of said numerical value. For example, the phrase “about 200” is used to mean a range ranging from and including 200+/−25%, i.e. ranging from and including 150 to 250; preferably 200+/−20%, i.e. ranging from and including 160 to 240; even more preferably ranging from and including 200+/−10%, i.e. ranging from and including 180 to 220. It is understood that a percentage given as “about 50%” does not mean “50%+/−25%”, i.e. ranging from and including 25 to 75%, but “about 50%” means ranging from and including 37.5 to 62.5%, i.e. plus and minus 25% of the numerical value which is 50.

As used herein, the term “polymer” means a molecule comprising repeating structural units, i.e. the monomers, connected by chemical bonds in a linear, circular, branched, crosslinked or dendrimeric way or a combination thereof, which may be of synthetic or biological origin or a combination of both. The monomers may be identical, in which case the polymer is a homopolymer, or may be different, in which case the polymer is a heteropolymer. A heteropolymer may also be referred to as a “copolymer” and includes for example alternating copolymers in which monomers of different types alternate; periodic copolymers in which monomers of different types of monomers are arranged in a repeating sequence; statistical copolymers in which monomers of different types are arranged randomly; block copolymers in which blocks of different homopolymers consisting of only one type of monomers are linked by a covalent bond; and gradient copolymers in which the composition of different monomers changes gradually along a polymer chain. It is understood that a polymer may also comprise one or more other moieties, such as, for example, one or more functional groups. Likewise, it is understood that also a peptide or protein is a polymer, even though the side chains of individual amino acid residues may be different. It is understood that for covalently crosslinked polymers, such as hydrogels, no meaningful molecular weight ranges can be provided.

As used herein, the term “polymeric” refers to a reagent or a moiety comprising one or more polymers or polymer moieties. A polymeric reagent or moiety may optionally also comprise one or more other moieties, which in certain embodiments are selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group comprising

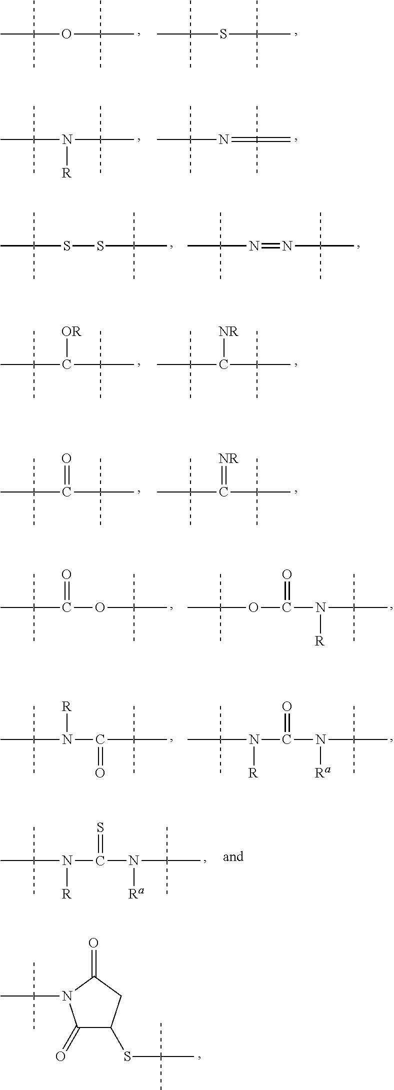

wherein
dashed lines indicate attachment to the remainder of the moiety or reagent, and —R and —R^{a} are independently of each other selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl;
which moieties and linkages are optionally further substituted.

The person skilled in the art understands that the polymerization products obtained from a polymerization reaction do not all have the same molecular weight, but rather exhibit a molecular weight distribution. Consequently, the molecular weight ranges, molecular weights, ranges of numbers of monomers in a polymer and numbers of monomers in a polymer as used herein, refer to the number average molecular weight and number average of monomers, i.e. to the arithmetic mean of the molecular weight of the polymer or polymeric moiety and the arithmetic mean of the number of monomers of the polymer or polymeric moiety.

Accordingly, in a polymeric moiety comprising “x” monomer units any integer given for “x” therefore corresponds to the arithmetic mean number of monomers. Any range of integers given for “x” provides the range of integers in which the arithmetic mean numbers of monomers lies. An integer for “x” given as “about x” means that the arithmetic mean numbers of monomers lies in a range of integers of x+/−25%, preferably x+/−20% and more preferably x+/−10%.

As used herein, the term “number average molecular weight” means the ordinary arithmetic mean of the molecular weights of the individual polymers.

As used herein, the term “PEG-based” in relation to a moiety or reagent means that said moiety or reagent comprises PEG. Such PEG-based moiety or reagent comprises at least 10% (w/w) PEG, such as at least 20% (w/w) PEG, such as at least 30% (w/w) PEG, such as at least 40% (w/w) PEG, such as at least 50% (w/w), such as at least 60 (w/w) PEG, such as at least 70% (w/w) PEG, such as at least 80% (w/w) PEG, such as at least 90% (w/w) PEG, or such as at least 95% (w/w) PEG. The remaining weight percentage of the PEG-based moiety or reagent may be other moieties, such as those selected from the group consisting of:
C₁₋₅₀ alkyl, C₂₋₅₀ alkenyl, C₂₋₅₀ alkynyl, C₃₋₁₀ cycloalkyl, 3- to 10-membered heterocyclyl, 8- to 11-membered heterobicyclyl, phenyl, naphthyl, indenyl, indanyl, and tetralinyl; and
linkages selected from the group consisting of

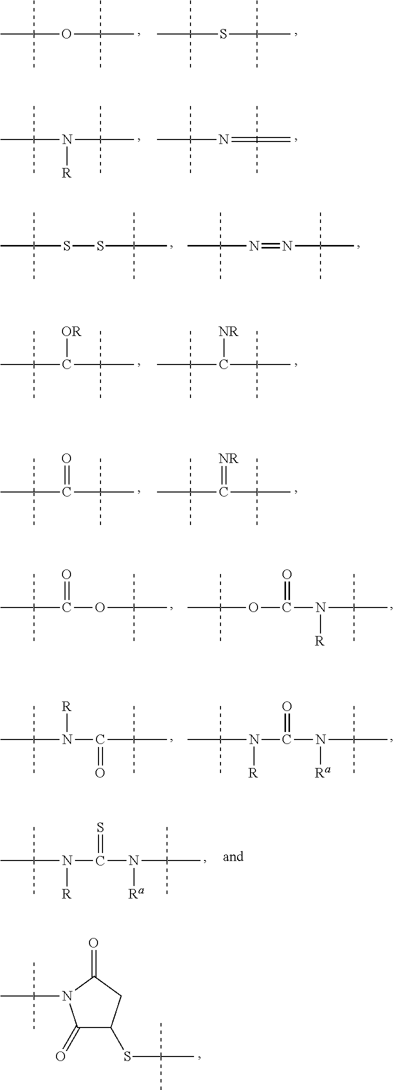

wherein
dashed lines indicate attachment to the remainder of the moiety or reagent, and —R and —R^{a} are independently of each other selected from the group consisting of —H, and C₁₋₆ alkyl; and
which moieties and linkages are optionally further substituted.

The term “interrupted” means that a moiety is inserted between two carbon atoms or—if the insertion is at one of the moiety's ends—between a carbon or heteroatom and a hydrogen atom.

As used herein, the term “C₁₋₄ alkyl” alone or in combination means a straight-chain or branched alkyl moiety having 1 to 4 carbon atoms. If present at the end of a molecule, examples of straight-chain or branched C₁₋₄ alkyl are methyl, ethyl, n-propyl, isopropyl, n-butyl, isobutyl, sec-butyl and tert-butyl. When two moieties of a molecule are linked by the C₁₋₄ alkyl, then examples for such C₁₋₄ alkyl groups are —CH₂—, —CH₂—CH₂—, —CH(CH₃)—, —CH₂—CH₂—CH₂—, —CH(C₂H₅)—, —C(CH₃)₂—. Each hydrogen of a C₁₋₄ alkyl carbon may optionally be replaced by a substituent as defined above. Optionally, a C₁₋₄ alkyl may be interrupted by one or more moieties as defined below.

As used herein, the term “C₁₋₆ alkyl” alone or in combination means a straight-chain or branched alkyl moiety having 1 to 6 carbon atoms. If present at the end of a molecule, examples of straight-chain and branched C₁₋₆ alkyl groups are methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. When two moieties of a molecule are linked by the C₁₋₆ alkyl group, then examples for such C₁₋₆ alkyl groups are —CH₂—, —CH₂—CH₂—, —CH(CH₃)—, —CH₂—CH₂—CH₂—,
—CH(C₂H₅)— and —C(CH₃)₂—. Each hydrogen atom of a C₁₋₆ carbon may optionally be replaced by a substituent as defined above. Optionally, a C₁₋₆ alkyl may be interrupted by one or more moieties as defined below.

Accordingly, “C₁₋₁₀ alkyl”, “C₁₋₂₀ alkyl” or “C₁₋₅₀ alkyl” means an alkyl chain having 1 to 10, 1 to 20 or 1 to 50 carbon atoms, respectively, wherein each hydrogen atom of the C₁₋₁₀, C₁₋₂₀ or C₁₋₅₀ carbon may optionally be replaced by a substituent as defined above. Optionally, a C₁₋₁₀ or C₁₋₅₀ alkyl may be interrupted by one or more moieties as defined below.

As used herein, the term “C₂₋₆ alkenyl” alone or in combination means a straight-chain or branched hydrocarbon moiety comprising at least one carbon-carbon double bond having 2 to 6 carbon atoms. If present at the end of a molecule, examples are —CH═CH₂, —CH═CH—CH₃, —CH₂—CH═CH₂, —CH═CHCH₂—CH₃ and —CH═CH—CH═CH₂. When two moieties of a molecule are linked by the C₂₋₆ alkenyl group, then an example for such C₂₋₆ alkenyl is —CH═CH—. Each hydrogen atom of a C₂₋₆ alkenyl moiety may optionally be replaced by a substituent as defined above. Optionally, a C₂₋₆ alkenyl may be interrupted by one or more moieties as defined below.

Accordingly, the terms “C₂₋₁₀ alkenyl”, “C₂₋₂₀ alkenyl” or “C₂₋₅₀ alkenyl” alone or in combination mean a straight-chain or branched hydrocarbon moiety comprising at least one carbon-carbon double bond having 2 to 10, 2 to 20 or 2 to 50 carbon atoms, respectively. Each hydrogen atom of a C₂₋₁₀ alkenyl, C₂₋₂₀ alkenyl or C₂₋₅₀ alkenyl group may optionally be replaced by a substituent as defined above. Optionally, a C₂₋₁₀ alkenyl, C₂₋₂₀ alkenyl or C₂₋₅₀ alkenyl may be interrupted by one or more moieties as defined below.

As used herein, the term “C₂₋₆ alkynyl” alone or in combination means a straight-chain or branched hydrocarbon moiety comprising at least one carbon-carbon triple bond having 2 to 6 carbon atoms. If present at the end of a molecule, examples are —C≡CH, —CH₂—C≡CH, CH₂—CH₂—C≡CH and CH₂—C≡C≡CH₃. When two moieties of a molecule are linked by the alkynyl group, then an example is —C≡C—. Each hydrogen atom of a C₂₋₆ alkynyl group may optionally be replaced by a substituent as defined above. Optionally, one or more double bond(s) may occur. Optionally, a C₂₋₆ alkynyl may be interrupted by one or more moieties as defined below.

Accordingly, as used herein, the term “C₂₋₁₀ alkynyl”, “C₂₋₂₀ alkynyl” and “C₂₋₅₀ alkynyl” alone or in combination means a straight-chain or branched hydrocarbon moiety comprising at least one carbon-carbon triple bond having 2 to 10, 2 to 20 or 2 to 50 carbon atoms, respectively. Each hydrogen atom of a C₂₋₁₀ alkynyl, C₂₋₂₀ alkynyl or C₂₋₅₀ alkynyl group may optionally be replaced by a substituent as defined above. Optionally, one or more double bond(s) may occur. Optionally, a C₂₋₁₀ alkynyl, C₂₋₂₀ alkynyl or C₂₋₅₀ alkynyl may be interrupted by one or more moieties as defined below.

As mentioned above, a C₁₋₄ alkyl, C₁₋₆ alkyl, C₁₋₁₀ alkyl, C₁₋₂₀ alkyl, C₁₋₅₀ alkyl, C₂₋₆ alkenyl, C₂₋₁₀ alkenyl, C₂₋₂₀ alkenyl, C₂₋₅₀ alkenyl, C₂₋₆ alkynyl, C₂₋₁₀ alkynyl, C₂₋₂₀ alkenyl or C₂₋₅₀ alkynyl may optionally be interrupted by one or more moieties which are preferably selected from the group consisting of

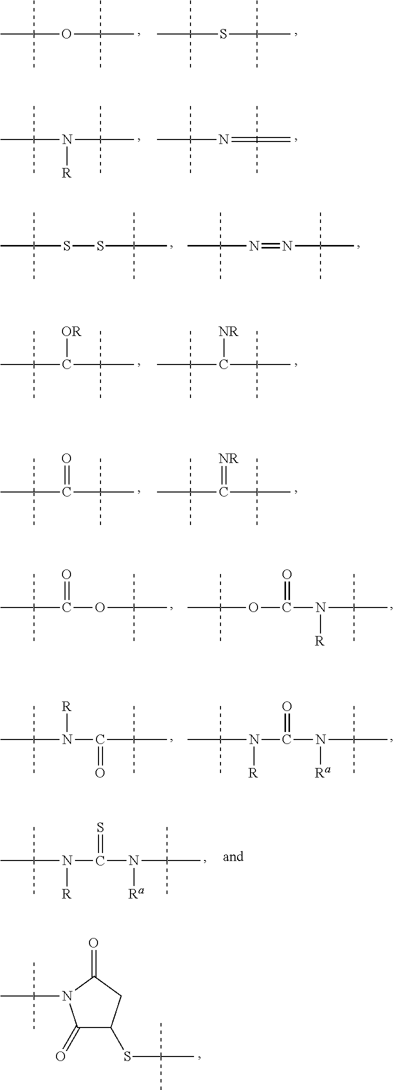

wherein
dashed lines indicate attachment to the remainder of the moiety or reagent; and —R and —R^{a} are independently of each other selected from the group consisting of —H and C₁₋₆ alkyl.

As used herein, the term “C₃₋₁₀ cycloalkyl” means a cyclic alkyl chain having 3 to 10 carbon atoms, which may be saturated or unsaturated, e.g. cyclopropyl, cyclobutyl, cyclopentyl, cyclohexyl, cyclohexenyl, cycloheptyl, cyclooctyl, cyclononyl or cyclodecyl. Each hydrogen atom of a C₃₋₁₀ cycloalkyl carbon may be replaced by a substituent as defined above. The term “C₃₋₁₀ cycloalkyl” also includes bridged bicycles like norbomane or norbomene.

The term “8- to 30-membered carbopolycyclyl” or “8- to 30-membered carbopolycycle” means a cyclic moiety of two or more rings with 8 to 30 ring atoms, where two neighboring rings share at least one ring atom and that may contain up to the maximum number of double bonds (aromatic or non-aromatic ring which is fully, partially or un-saturated). Preferably a 8- to 30-membered carbopolycyclyl means a cyclic moiety of two, three, four or five rings, more preferably of two, three or four rings.

As used herein, the term “3- to 10-membered heterocyclyl” or “3- to 10-membered heterocycle” means a ring with 3, 4, 5, 6, 7, 8, 9 or 10 ring atoms that may contain up to the maximum number of double bonds (aromatic or non-aromatic ring which is fully, partially or un-saturated) wherein at least one ring atom up to 4 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for 3- to 10-membered heterocycles include but are not limited to aziridine, oxirane, thiirane, azirine, oxirene, thiirene, azetidine, oxetane, thietane, furan, thiophene, pyrrole, pyrroline, imidazole, imidazoline, pyrazole, pyrazoline, oxazole, oxazoline, isoxazole, isoxazoline, thiazole, thiazoline, isothiazole, isothiazoline, thiadiazole, thiadiazoline, tetrahydrofuran, tetrahydrothiophene, pyrrolidine, imidazolidine, pyrazolidine, oxazolidine, isoxazolidine, thiazolidine, isothiazolidine, thiadiazolidine, sulfolane, pyran, dihydropyran, tetrahydropyran, imidazolidine, pyridine, pyridazine, pyrazine, pyrimidine, piperazine, piperidine, morpholine, tetrazole, triazole, triazolidine, tetrazolidine, diazepane, azepine and homopiperazine. Each hydrogen atom of a 3- to 10-membered heterocyclyl or 3- to 10-membered heterocyclic group may be replaced by a substituent.

As used herein, the term “8- to 11-membered heterobicyclyl” or “8- to 11-membered heterobicycle” means a heterocyclic moiety of two rings with 8 to 11 ring atoms, where at least one ring atom is shared by both rings and that may contain up to the maximum number of double bonds (aromatic or non-aromatic ring which is fully, partially or un-saturated) wherein at least one ring atom up to 6 ring atoms are replaced by a heteroatom selected from the group consisting of sulfur (including —S(O)—, —S(O)₂—), oxygen and nitrogen (including ═N(O)—) and wherein the ring is linked to the rest of the molecule via a carbon or nitrogen atom. Examples for an 8- to 11-membered heterobicycle are indole, indoline, benzofuran, benzothiophene, benzoxazole, benzisoxazole, benzothiazole, benzisothiazole, benzimidazole, benzimidazoline, quinoline, quinazoline, dihydroquinazoline, quinoline, dihydroquinoline, tetrahydroquinoline, decahydroquinoline, isoquinoline, decahydroisoquinoline, tetrahydroisoquinoline, dihydroisoquinoline, benzazepine, purine and pteridine. The term 8- to 11-membered heterobicycle also includes spiro structures of two rings like 1,4-dioxa-8-azaspiro[4.5]decane or bridged heterocycles like 8-aza-bicyclo[3.2.1]octane. Each hydrogen atom of an 8- to 11-membered heterobicyclyl or 8- to 11-membered heterobicycle carbon may be replaced by a substituent.

Similary, the term “8- to 30-membered heteropolycyclyl” or “8- to 30-membered heteropolycycle” means a heterocyclic moiety of more than two rings with 8 to 30 ring atoms, preferably of three, four or five rings, where two neighboring rings share at least one ring atom and that may contain up to the maximum number of double bonds (aromatic or non-aromatic ring which is fully, partially or unsaturated), wherein at least one ring atom up to 10 ring atoms are replaced by a heteroatom selected from the group of sulfur (including —S(O)—, —S(O)₂—), oxygen and nitrogen (including ═N(O)—) and wherein the ring is linked to the rest of a molecule via a carbon or nitrogen atom.

It is understood that the phrase “the pair R^{x}/R^{y} is joined together with the atom to which they are attached to form a C₃₋₁₀ cycloalkyl or a 3- to 10-membered heterocyclyl” in relation with a moiety of the structure

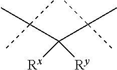

means that R^{x} and R^{y} form the following structure:

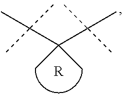

wherein R is a C₃₋₁₀ cycloalkyl or 3- to 10-membered heterocyclyl.

It is also understood that the phrase “the pair R^{x}/R^{y} is joint together with the atoms to which they are attached to form a ring A” in relation with a moiety of the structure

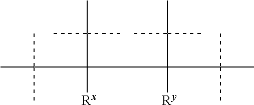

means that R^{x} and R^{y} form the following structure:

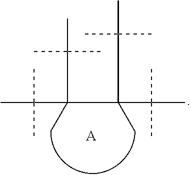

It is also understood that the phrase “—R¹ and an adjacent —R² form a carbon-carbon double bond provided that n is selected from the group consisting of 1, 2, 3 and 4” in relation with a moiety of the structure:

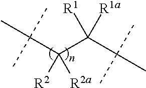

means that for example when n is 1, —R¹ and the adjacent —R² form the following structure:

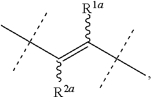

and if for example, n is 2, R¹ and the adjacent —R² form the following structure:

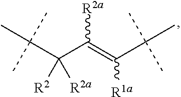

wherein the wavy bond means that —R^{1a} and —R^{2a} may be either on the same side of the double bond, i.e. in cis configuration, or on opposite sides of the double bond, i.e. in trans configuration and wherein the term “adjacent” means that —R¹ and —R² are attached to carbon atoms that are next to each other.

It is also understood that the phrase “two adjacent —R² form a carbon-carbon double bond provided that n is selected from the group consisting of 2, 3 and 4” in relation with a moiety of the structure:

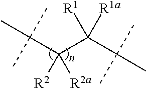

means that for example when n is 2, two adjacent —R² form the following structure:

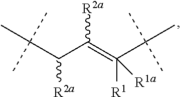

wherein the wavy bond means that each —R^{2a} may be either on the same side of the double bond, i.e. in cis configuration, or on opposite sides of the double bond, i.e. in trans configuration and wherein the term “adjacent” means that two —R² are attached to carbon atoms that are next to each other.

It is understood that the “N” in the phrase “π-electron-pair-donating heteroaromatic N” refers to nitrogen.

It is understood that “N⁺” in the phrases “an electron-donating heteroaromatic N⁺-comprising moiety” and “attachment to the N⁺ of -D⁺” refers to a positively charged nitrogen atom.

As used herein, “halogen” means fluoro, chloro, bromo or iodo. In certain embodiments halogen is fluoro or chloro.

As used herein, the term “functional group” means a group of atoms which can react with other groups of atoms. Exemplary functional groups are carboxylic acid, primary amine, secondary amine, tertiary amine, maleimide, thiol, sulfonic acid, carbonate, carbamate, hydroxyl, aldehyde, ketone, hydrazine, isocyanate, isothiocyanate, phosphoric acid, phosphonic acid, haloacetyl, alkyl halide, acryloyl, aryl fluoride, hydroxylamine, disulfide, sulfonamides, sulfuric acid, vinyl sulfone, vinyl ketone, diazoalkane, oxirane, and aziridine.

In case the conjugates of the present invention comprise one or more acidic or basic groups, the invention also comprises their corresponding pharmaceutically or toxicologically acceptable salts, in particular their pharmaceutically utilizable salts. Thus, the conjugates of the present invention comprising acidic groups can be used according to the invention, for example, as alkali metal salts, alkaline earth metal salts or as ammonium salts. More precise examples of such salts include sodium salts, potassium salts, calcium salts, magnesium salts or salts with ammonia or organic amines such as, for example, ethylamine, ethanolamine, triethanolamine or amino acids, or quaternary ammoniums, such as tetrabutylammonium and cetyl trimethylammonium. Conjugates of the present invention comprising one or more basic groups, i.e. groups which can be protonated, can be present and can be used according to the invention in the form of their addition salts with inorganic or organic acids. Examples for suitable acids include hydrogen chloride, hydrogen bromide, phosphoric acid, sulfuric acid, nitric acid, methanesulfonic acid, p-toluenesulfonic acid, naphthalenedisulfonic acids, oxalic acid, acetic acid, tartaric acid, lactic acid, salicylic acid, benzoic acid, formic acid, propionic acid, pivalic acid, diethylacetic acid, malonic acid, succinic acid, pimelic acid, fumaric acid, maleic acid, malic acid, sulfaminic acid, phenylpropionic acid, gluconic acid, ascorbic acid, isonicotinic acid, citric acid, adipic acid, trifluoroacetic acid and other acids known to the person skilled in the art. For the person skilled in the art further methods are known for converting the basic group into a cation like the alkylation of an amine group resulting in a positively-charge ammonium group and an appropriate counterion of the salt. If the conjugates of the present invention simultaneously comprise acidic and basic groups, the invention also includes, in addition to the salt forms mentioned, inner salts or betaines (zwitterions). The respective salts can be obtained by customary methods, which are known to the person skilled in the art like, for example by contacting these prodrugs with an organic or inorganic acid or base in a solvent or dispersant, or by anion exchange or cation exchange with other salts. The present invention also includes all salts of the conjugates of the present invention which, owing to low physiological compatibility, are not directly suitable for use in pharmaceuticals but which can be used, for example, as intermediates for chemical reactions or for the preparation of pharmaceutically acceptable salts.

The term “pharmaceutically acceptable” means a substance that does not cause harm when administered to a patient and preferably means approved by a regulatory agency, such as the EMA (Europe) and/or the FDA (US) and/or any other national regulatory agency for use in animals, preferably for use in humans.

As used herein, the term “excipient” refers to a diluent, adjuvant, or vehicle with which the therapeutic, such as a drug or prodrug, is administered. Such pharmaceutical excipient can be sterile liquids, such as water and oils, including those of petroleum, animal, vegetable or synthetic origin, including but not limited to peanut oil, soybean oil, mineral oil, sesame oil and the like. Water is a preferred excipient when the pharmaceutical composition is administered orally. Saline and aqueous dextrose are preferred excipients when the pharmaceutical composition is administered intravenously. Saline solutions and aqueous dextrose and glycerol solutions are preferably employed as liquid excipients for injectable solutions. Suitable pharmaceutical excipients include starch, glucose, lactose, sucrose, mannitol, trehalose, gelatin, malt, rice, flour, chalk, silica gel, sodium stearate, glycerol monostearate, talc, sodium chloride, dried skim milk, glycerol, propylene, glycol, hyaluronic acid, propylene glycol, water, ethanol and the like. The pharmaceutical composition, if desired, can also contain minor amounts of wetting or emulsifying agents, pH buffering agents, like, for example, acetate, succinate, tris, carbonate, phosphate, HEPES (4-(2-hydroxyethyl)-1-piperazineethanesulfonic acid), MES (2-(N-morpholino)ethanesulfonic acid), or can contain detergents, like Tween, poloxamers, poloxamines, CHAPS, Igepal, or amino acids like, for example, glycine, lysine, or histidine. These pharmaceutical compositions can take the form of solutions, suspensions, emulsions, tablets, pills, capsules, powders, sustained-release formulations and the like. The pharmaceutical composition can be formulated as a suppository, with traditional binders and excipients such as triglycerides. Oral formulation can include standard excipients such as pharmaceutical grades of mannitol, lactose, starch, magnesium stearate, sodium saccharine, cellulose, magnesium carbonate, etc. Such compositions will contain a therapeutically effective amount of the drug or drug moiety, together with a suitable amount of excipient so as to provide the form for proper administration to the patient. The formulation should suit the mode of administration.

The term “peptide” as used herein refers to a chain of at least 2 and up to and including 50 amino acid monomer moieties, which may also be referred to as “amino acid residues”, linked by peptide (amide) linkages. The amino acid monomers may be selected from the group consisting of proteinogenic amino acids and non-proteinogenic amino acids and may be D- or L-amino acids. The term “peptide” also includes peptidomimetics, such as peptoids, beta-peptides, cyclic peptides and depsipeptides and covers such peptidomimetic chains with up to and including 50 monomer moieties.

As used herein, the term “protein” refers to a chain of more than 50 amino acid monomer moieties, which may also be referred to as “amino acid residues”, linked by peptide linkages, in which preferably no more than 12000 amino acid monomers are linked by peptide linkages, such as no more than 10000 amino acid monomer moieties, no more than 8000 amino acid monomer moieties, no more than 5000 amino acid monomer moieties or no more than 2000 amino acid monomer moieties.

As used herein, the term “oligonucleotide” refers to a nucleic acid polymer of up to 100 bases and may be both DNA and RNA. The term also includes aptamers and morpholinos.

As used herein the term “small molecule drug” refers to drugs that are organic compounds with a molecular weight of no more than 1 kDa, such as up to 900 kDa.

As used herein the term “antibiotic” refers to an antimicrobial drug for the treatment or prevention of bacterial infections, which either kills or inhibits growth of bacteria. The term also refers to drugs having antiprotozoal and antifungal activity.

As used herein, the term “biofilm” refers to a plurality of microorganisms, such as microorganisms selected from the group consisting of bacteria, archaea, protozoa, fungi and algae, such as to a plurality of bacteria, embedded within an extracellular matrix that is composed of extracellular polymeric substances, such as polysaccharides, proteins and DNA, and said extracellular matrix may comprise material from the surrounding environment, such as blood components. Biofilms may form on living and non-living surfaces and may comprise one or more species of microorganism. It is known that during the ageing process of a biofilm it becomes increasingly difficult to eradicate it, because not only do individual cells form tighter bonds with the surface, but the extracellular matrix also provides a protective environment that restricts access of the antibiotics to the microorganisms.

As used herein the terms “compartment” and “body compartment” are used synonymously and refer to any particular space in the body comprising a diffusion barrier impeding the exchange of solutes with the surrounding tissue. Such space may also be artificially introduced by, for example, an implant. This space may be fluid, solid or may contain a gas phase or may be any combination thereof. It is understood that said solutes may comprise pharmacologically active compounds. The terms “compartment” and “body compartment” also refer to body structures that are separated by membranes, sheaths, linings, fascia and other connective tissue, bones, cartilage, or any combination thereof.

As used herein the term “pattern recognition receptor agonist” (“PRRA”) refers to a molecule that binds to and activates one or more immune cell-associated receptor that recognizes pathogen-associated molecular patterns (PAMPs) or damage-associated molecular patterns (DAMPs), leading to immune cell activation and/or pathogen- or damage-induced inflammatory responses. PRRs are typically expressed by cells of the innate immune system such as monocytes, macrophages, dendritic cells (DCs), neutrophils, and epithelial cells, as well as cells of the adaptive immune system.

As used herein the term “tyrosine kinase inhibitor” or “TKI” refers to a molecule that binds to and inhibits one or more cell-associated receptor or non-receptor tyrosine kinases that are activated via polypeptide growth factors, cytokines, hormones, or phosphorylation, and are involved in cellular signaling, cellular development, cellular proliferation, cellular maturation, cellular metabolism, angiogenesis, and in certain instances, tumorigenesis. Tyrosine kinases are ubiquitously expressed by virtually all cells. TKIs inhibit activation of tyrosine kinases by multiple mechanisms such as competing with, or allosterically antagonizing, binding of adenosine triphosphate (ATP) to the tyrosine kinase ATP-binding site, or by inhibiting enzymatic phosphorylation of said binding site, or inhibiting enzymatic kinase activity. In the case of receptor tyrosine kinases (RTKs), receptor TKIs may bind one or more RTKs and inhibit RTK activation as described above or by antagonizing activating ligand interactions, thus preventing receptor tyrosine kinase activation.

As used herein the terms “anti-CTLA4 drug” and “anti-CTLA4 moiety” refer to a drug or moiety, respectively, which binds to CTLA4 and which may block the interaction with its ligands B7.1 and B7.2 (CD80 and CD86). In certain embodiments such anti-CTLA4 drug or anti-CTLA4 moiety may be selected from the group consisting of antibodies, antibody fragments, affibodies, affilins, affimers, affitins, alphamabs, alphabodies, anticalins, avimers, DARPins, Fynomers®, Kunitz domain peptides, monobodies, nanoCLAMPs, cyclic peptides, small molecules and nanobodies.

In general, the terms “comprise” or “comprising” also encompasses “consist of” or “consisting of”.

In certain embodiments the crosslinker moiety has a molecular weight ranging from 0.2 kDa to 25 kDa, such as from 1 kDa to 10 kDa or from 1.5 kDa to 5 kDa. In certain embodiments the crosslinker has a molecular weight of 1 kDa. In certain embodiments the crosslinker has a molecular weight of 1.2 kDa. In certain embodiments the crosslinker has a molecular weight of 1.4 kDa. In certain embodiments the crosslinker has a molecular weight of 1.5 kDa. In certain embodiments the crosslinker has a molecular weight of 1.8 kDa. In certain embodiments the crosslinker has a molecular weight of 2 kDa. In certain embodiments the crosslinker has a molecular weight of 2.2 kDa. In certain embodiments the crosslinker has a molecular weight of 2.4 kDa. In certain embodiments the crosslinker has a molecular weight of 2.5 kDa. In certain embodiments the crosslinker has a molecular weight of 2.8 kDa. In certain embodiments the crosslinker has a molecular weight of 3 kDa. In certain embodiments the crosslinker has a molecular weight of 3.3 kDa. In certain embodiments the crosslinker has a molecular weight of 3.5 kDa. In certain embodiments the crosslinker has a molecular weight of 3.8 kDa. In certain embodiments the crosslinker has a molecular weight of 4 kDa. In certain embodiments the crosslinker has a molecular weight of 4.2 kDa. In certain embodiments the crosslinker has a molecular weight of 4.5 kDa. In certain embodiments the crosslinker has a molecular weight of 4.8 kDa. In certain embodiments the crosslinker has a molecular weight of 5 kDa.

In certain embodiment s3 of formula (A) ranges from 1 to 500. In certain embodiments s3 of formula (A) ranges from 1 to 200.

In certain embodiments r1 of formula (A) is 0. In certain embodiments r1 of formula (A) is 1. In certain embodiments r2 of formula (A) is 0. In certain embodiments r2 of formula (A) is 1. In certain embodiments r5 of formula (A) is 0. In certain embodiments r5 of formula (A) is 1. In certain embodiments r6 of formula (A) is 0. In certain embodiments r6 of formula (A) is 1.

In certain embodiments r1, r2, r5 and r6 of formula (A) are 0.

In certain embodiments r13 of formula (A) is 0. In certain embodiments r13 of formula (A) is 1. In certain embodiments r14 of formula (A) is 0. In certain embodiments r14 of formula (A) is 1. In certain embodiments r15 of formula (A) is 0. In certain embodiments r15 of formula (A) is 1. In certain embodiments r16 of formula (A) is 0. In certain embodiments r16 of formula (A) is 1.

In certain embodiments r3 of formula (A) is 1. In certain embodiments r3 of formula (A) is 2. In certain embodiments r4 of formula (A) is 1. In certain embodiments r4 of formula (A) is 2. In certain embodiments r3 and r4 of formula (A) are both 1. In certain embodiments r3 and r4 of formula (A) are both 2. In certain embodiments r3 and r4 of formula (A) are both 3.

In certain embodiments r7 of formula (A) is 0. In certain embodiments r7 of formula (A) is 1. In certain embodiments r7 of formula (A) is 2. In certain embodiments r8 of formula (A) is 0. In certain embodiments r8 of formula (A) is 1. In certain embodiments r8 of formula (A) is 2. In certain embodiments r9 of formula (A) is 0. In certain embodiments r9 of formula (A) is 1. In certain embodiments r9 of formula (A) is 2. In certain embodiments r10 of formula (A) is 0. In certain embodiments r10 of formula (A) is 1. In certain embodiments r10 of formula (A) is 2. In certain embodiments r11 of formula (A) is 0. In certain embodiments r11 of formula (A) is 1. In certain embodiments r11 of formula (A) is 2. In certain embodiments r12 of formula (A) is 0. In certain embodiments r12 of formula (A) is 1. In certain embodiments r12 of formula (A) is 2.

In certain embodiments r17 of formula (A) is 1. In certain embodiments r18 of formula (A) is 1. In certain embodiments r19 of formula (A) is 1. In certain embodiments r20 of formula (A) is 1. In certain embodiments r21 of formula (A) is 1. In certain embodiments r22 of formula (A) is 1.

In certain embodiments s1 of formula (A) is 1. In certain embodiments s1 of formula (A) is 2. In certain embodiments s2 of formula (A) is 1. In certain embodiments s2 of formula (A) is 2. In certain embodiments s4 of formula (A) is 1. In certain embodiments s4 of formula (A) is 2. In certain embodiments s5 of formula (A) is 1. In certain embodiments s5 of formula (A) is 2.

In certain embodiments s3 of formula (A) ranges from 5 to 500. In certain embodiments s3 of formula (A) ranges from 10 to 250. In certain embodiments s3 of formula (A) ranges from 12 to 150. In certain embodiments s3 of formula (A) ranges from 15 to 100. In certain embodiments s3 of formula (A) ranges from 18 to 75. In certain embodiments s3 of formula (A) ranges from 20 to 50.

In certain embodiments —R¹ of formula (A) is —H. In certain embodiments —R¹ of formula (A) is methyl. In certain embodiments —R¹ of formula (A) is ethyl. In certain embodiments —R^{1a} of formula (A) is —H. In certain embodiments —R^{1a} of formula (A) is methyl. In certain embodiments —R^{1a} of formula (A) is ethyl. In certain embodiments —R² of formula (A) is —H. In certain embodiments —R² of formula (A) is methyl. In certain embodiments —R² of formula (A) is ethyl. In certain embodiments —R^{2a} of formula (A) is —H. In certain embodiments —R^{2a} of formula (A) is methyl. In certain embodiments —R^{2a} of formula (A) is ethyl. In certain embodiments —R³ of formula (A) is —H. In certain embodiments —R³ of formula (A) is methyl. In certain embodiments —R³ of formula (A) is ethyl. In certain embodiments —R^{3a} of formula (A) is —H. In certain embodiments —R^{3a} of formula (A) is methyl. In certain embodiments —R^{3a} of formula (A) is ethyl. In certain embodiments —R⁴ of formula (A) is —H. In certain embodiments —R⁴ of formula (A) is methyl. In certain embodiments —R⁴ of formula (A) is methyl. In certain embodiments —R^{4a} of formula (A) is —H. In certain embodiments —R^{4a} of formula (A) is methyl. In certain embodiments —R^{4a} of formula (A) is ethyl. In certain embodiments —R⁵ of formula (A) is —H. In certain embodiments —R⁵ of formula (A) is methyl. In certain embodiments —R⁵ of formula (A) is ethyl. In certain embodiments —R^{5a} of formula (A) is —H. In certain embodiments —R^{5a} of formula (A) is methyl. In certain embodiments —R^{5a} of formula (A) is ethyl. In certain embodiments —R⁶ of formula (A) is —H. In certain embodiments —R⁶ of formula (A) is methyl. In certain embodiments —R⁶ of formula (A) is ethyl. In certain embodiments —R^{6a} of formula (A) is —H. In certain embodiments —R^{6a} of formula (A) is methyl. In certain embodiments —R^{6a} of formula (A) is ethyl. In certain embodiments —R⁷ of formula (A) is —H. In certain embodiments —R⁷ of formula (A) is methyl. In certain embodiments —R⁷ of formula (A) is ethyl. In certain embodiments —R^{7a} of formula (A) is —H. In certain embodiments —R^{7a} of formula (A) is methyl. In certain embodiments —R^{7a} of formula (A) is ethyl. In certain embodiments —R⁸ of formula (A) is —H. In certain embodiments —R⁸ of formula (A) is methyl. In certain embodiments —R⁸ of formula (A) is ethyl. In certain embodiments —R^{8a} of formula (A) is —H. In certain embodiments —R^{8a} of formula (A) is methyl. In certain embodiments —R^{8a} of formula (A) is ethyl. In certain embodiments —R⁹ of formula (A) is —H. In certain embodiments —R⁹ of formula (A) is methyl. In certain embodiments —R⁹ of formula (A) is ethyl. In certain embodiments —R^{9a} of formula (A) is —H. In certain embodiments —R^{9a} of formula (A) is methyl. In certain embodiments —R^{9a} of formula (A) is ethyl In certain embodiments —R¹⁰ of formula (A) is —H. In certain embodiments —R¹⁰ of formula (A) is methyl. In certain embodiments —R¹⁰ of formula (A) is ethyl. In certain embodiments —R^{10a} of formula (A) is —H. In certain embodiments —R^{10a} of formula (A) is methyl. In certain embodiments —R^{10a} of formula (A) is ethyl. In certain embodiments —R¹¹ of formula (A) is —H. In certain embodiments —R¹¹ of formula (A) is methyl. In certain embodiments —R¹¹ of formula (A) is ethyl. In certain embodiments —R¹² of formula (A) is —H. In certain embodiments —R¹² of formula (A) is methyl. In certain embodiments —R¹² of formula (A) is ethyl. In certain embodiments —R^{12a} of formula (A) is —H. In certain embodiments —R^{12a} of formula (A) is methyl. In certain embodiments —R^{12a} of formula (A) is ethyl. In certain embodiments —R¹³ of formula (A) is —H. In certain embodiments —R¹³ of formula (A) is methyl. In certain embodiments —R¹³ of formula (A) is ethyl. In certain embodiments —R¹⁴ of formula (A) is —H. In certain embodiments —R¹⁴ of formula (A) is methyl. In certain embodiments —R¹⁴ of formula (A) is ethyl. In certain embodiments —R^{14a} of formula (A) is —H. In certain embodiments —R^{14a} of formula (A) is methyl. In certain embodiments —R^{14a} of formula (A) is ethyl.

In certain embodiments -D¹- of formula (A) is —O—. In certain embodiments -D¹- of formula (A) is —NR¹¹—. In certain embodiments -D¹- of formula (A) is —N⁺R¹²R^{12a}—. In certain embodiments -D¹- of formula (A) is —S—. In certain embodiments -D¹- of formula (A) is —(S═O). In certain embodiments -D¹- of formula (A) is —(S(O)₂)—. In certain embodiments -D¹- of formula (A) is —C(O)—. In certain embodiments -D¹- of formula (A) is —P(O)R¹³—. In certain embodiments -D¹- of formula (A) is —P(O)(OR¹³)—. In certain embodiments -D¹- of formula (A) is —CR¹⁴R^{14a}—.

In certain embodiments -D²- of formula (A) is —O—. In certain embodiments -D²- of formula (A) is —NR¹¹—. In certain embodiments -D²- of formula (A) is —N⁺R¹²R^{12a}—. In certain embodiments -D²- of formula (A) is —S—. In certain embodiments -D²- of formula (A) is —(S═O). In certain embodiments -D²- of formula (A) is —(S(O)₂)—. In certain embodiments -D²- of formula (A) is —C(O)—. In certain embodiments -D²- of formula (A) is —P(O)R¹³—. In certain embodiments -D²- of formula (A) is —P(O)(OR¹³)—. In certain embodiments -D²- of formula (A) is —CR¹⁴R^{14a}—.

In certain embodiments -D³- of formula (A) is —O—. In certain embodiments -D³- of formula (A) is —NR¹¹—. In certain embodiments -D³- of formula (A) is —N⁺R¹²R^{12a}—. In certain embodiments -D³- of formula (A) is —S—. In certain embodiments -D³- of formula (A) is —(S═O). In certain embodiments -D³- of formula (A) is —(S(O)₂)—. In certain embodiments -D³- of formula (A) is —C(O)—. In certain embodiments -D³- of formula (A) is —P(O)R¹³—. In certain embodiments -D³- of formula (A) is —P(O)(OR¹³)—. In certain embodiments -D³- of formula (A) is —CR¹⁴R^{14a}—.

In certain embodiments -D⁴- of formula (A) is —O—. In certain embodiments -D⁴- of formula (A) is —NR¹¹—. In certain embodiments -D⁴- of formula (A) is —N⁺R¹²R^{12a}—. In certain embodiments -D⁴- of formula (A) is —S—. In certain embodiments -D⁴- of formula (A) is —(S═O). In certain embodiments -D⁴- of formula (A) is —(S(O)₂)—. In certain embodiments -D⁴- of formula (A) is —C(O)—. In certain embodiments -D⁴- of formula (A) is —P(O)R¹³—. In certain embodiments -D⁴- of formula (A) is —P(O)(OR¹³)—. In certain embodiments -D⁴- of formula (A) is —CR¹⁴R^{14a}—.

In certain embodiments -D⁵- of formula (A) is —O—. In certain embodiments -D⁵- of formula (A) is —NR¹¹—. In certain embodiments -D⁵- of formula (A) is —N⁺R¹²R^{12a}—. In certain embodiments -D⁵- of formula (A) is —S—. In certain embodiments -D⁵- of formula (A) is —(S═O)—. In certain embodiments -D⁵- of formula (A) is —(S(O)₂)—. In certain embodiments -D⁵- of formula (A) is —C(O)—. In certain embodiments -D⁵- of formula (A) is —P(O)R¹³—. In certain embodiments -D⁵- of formula (A) is —P(O)(OR¹³)—. In certain embodiments -D⁵- of formula (A) is —CR¹⁴R^{14a}—.

In certain embodiments -D⁶- of formula (A) is —O—. In certain embodiments -D⁶- of formula (A) is —NR¹¹—. In certain embodiments -D⁶- of formula (A) is —N⁺R¹²R^{12a}—. In certain embodiments -D⁶- of formula (A) is —S—. In certain embodiments -D⁶- of formula (A) is —(S═O). In certain embodiments -D⁶- of formula (A) is —(S(O)₂)—. In certain embodiments -D⁶- of formula (A) is —C(O)—. In certain embodiments -D⁶- of formula (A) is —P(O)R¹³—. In certain embodiments -D⁶- of formula (A) is —P(O)(OR¹³)—. In certain embodiments -D⁶- of formula (A) is —CR¹⁴R^{14a}—.

In certain embodiments —CL- is of formula (A-i)

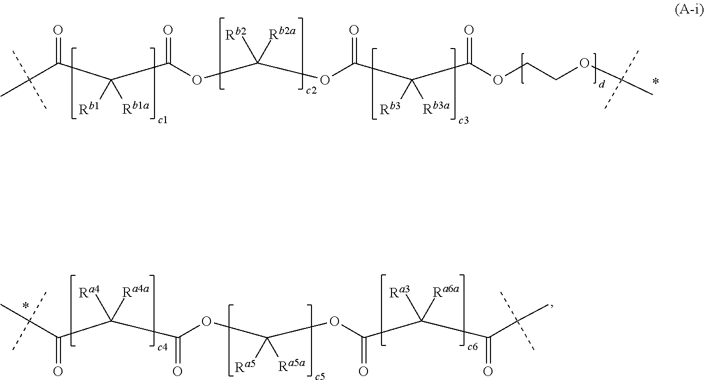

wherein
dashed lines marked with an asterisk indicate the connection point between the upper and the lower substructure, unmarked dashed lines indicate attachment to a backbone moiety or to a spacer moiety —SP—;
—R^{b1}, —R^{b1a}, —R^{b2}, —R^{b2a}, —R^{b3}, —R^{b3a}, —R^{b4}, —R^{b4a}, —R^{b5}, —R^{b5a}, —R^{b6} and —R^{b6} are independently selected from the group consisting of —H and C₁₋₆ alkyl;
c1, c2, c3, c4, c5 and c6 are independently selected from the group consisting of 1, 2, 3, 4, 5 and 6;
d is an integer ranging from 2 to 250.

In certain embodiments d of formula (A-i) ranges from 3 to 200. In certain embodiments d of formula (A-i) ranges from 4 to 150. In certain embodiments d of formula (A-i) ranges from 5 to 100. In certain embodiments d of formula (A-i) ranges from 10 to 50. In certain embodiments d of formula (A-i) ranges from 15 to 30. In certain embodiments d of formula (A-i) is about 23.

In certain embodiments —R^{b1} and —R^{b1a} of formula (A-i) are —H. In certain embodiments —R^{b2} and —R^{b2a} of formula (A-i) are —H. In certain embodiments —R^{b3} and —R^{b3a} of formula (A-i) are —H. In certain embodiments —R^{b4} and —R^{b4a} of formula (A-i) are —H. In certain embodiments —R^{b5} and —R^{b5a} of formula (A-i) are —H. In certain embodiments —R^{b6} and —R^{b6a} of formula (A-i) are —H.

In certain embodiments —R^{b1}, —R^{b1a}, —R^{b2}, —R^{b2a}, —R^{b3}, —R^{b3a}, —R^{b4}, —R^{b4a}, —R^{b5}, —R^{b5a}, —R^{b6} and —R^{b6} of formula (A-i) are all —H.

In certain embodiments c1 of formula (A-i) is 1. In certain embodiments c1 of formula (A-i) is 2. In certain embodiments c1 of formula (A-i) is 3. In certain embodiments c1 of formula (A-i) is 4. In certain embodiments c1 of formula (A-i) is 5. In certain embodiments c1 of formula (A-i) is 6.

In certain embodiments c2 of formula (A-i) is 1. In certain embodiments c2 of formula (A-i) is 2. In certain embodiments c2 of formula (A-i) is 3. In certain embodiments c2 of formula (A-i) is 4. In certain embodiments c2 of formula (A-i) is 5. In certain embodiments c2 of formula (A-i) is 6.

In certain embodiments c3 of formula (A-i) is 1. In certain embodiments c3 of formula (A-i) is 2. In certain embodiments c3 of formula (A-i) is 3. In certain embodiments c3 of formula (A-i) is 4. In certain embodiments c3 of formula (A-i) is 5. In certain embodiments c3 of formula (A-i) is 6.

In certain embodiments c4 of formula (A-i) is 1. In certain embodiments c4 of formula (A-i) is 2. In certain embodiments c4 of formula (A-i) is 3. In certain embodiments c4 of formula (A-i) is 4. In certain embodiments c4 of formula (A-i) is 5. In certain embodiments c4 of formula (A-i) is 6.

In certain embodiments c5 of formula (A-i) is 1. In certain embodiments c5 of formula (A-i) is 2. In certain embodiments c5 of formula (A-i) is 3. In certain embodiments c5 of formula (A-i) is 4. In certain embodiments c5 of formula (A-i) is 5. In certain embodiments c5 of formula (A-i) is 6.

In certain embodiments c6 of formula (A-i) is 1. In certain embodiments c6 of formula (A-i) is 2. In certain embodiments c6 of formula (A-i) is 3. In certain embodiments c6 of formula (A-i) is 4. In certain embodiments c6 of formula (A-i) is 5. In certain embodiments c6 of formula (A-i) is 6.

In certain embodiments a crosslinker moiety —CL- is of formula (A-i1)

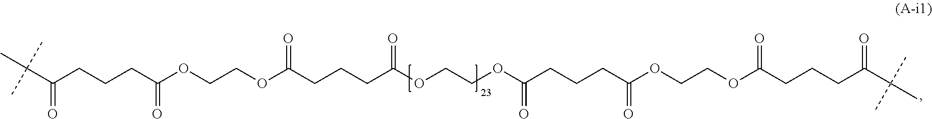

wherein
dashed lines indicate attachment to a backbone moiety or to a spacer moiety —SP—.

In certain embodiments a crosslinker moiety —CL- is selected from the group consisting of

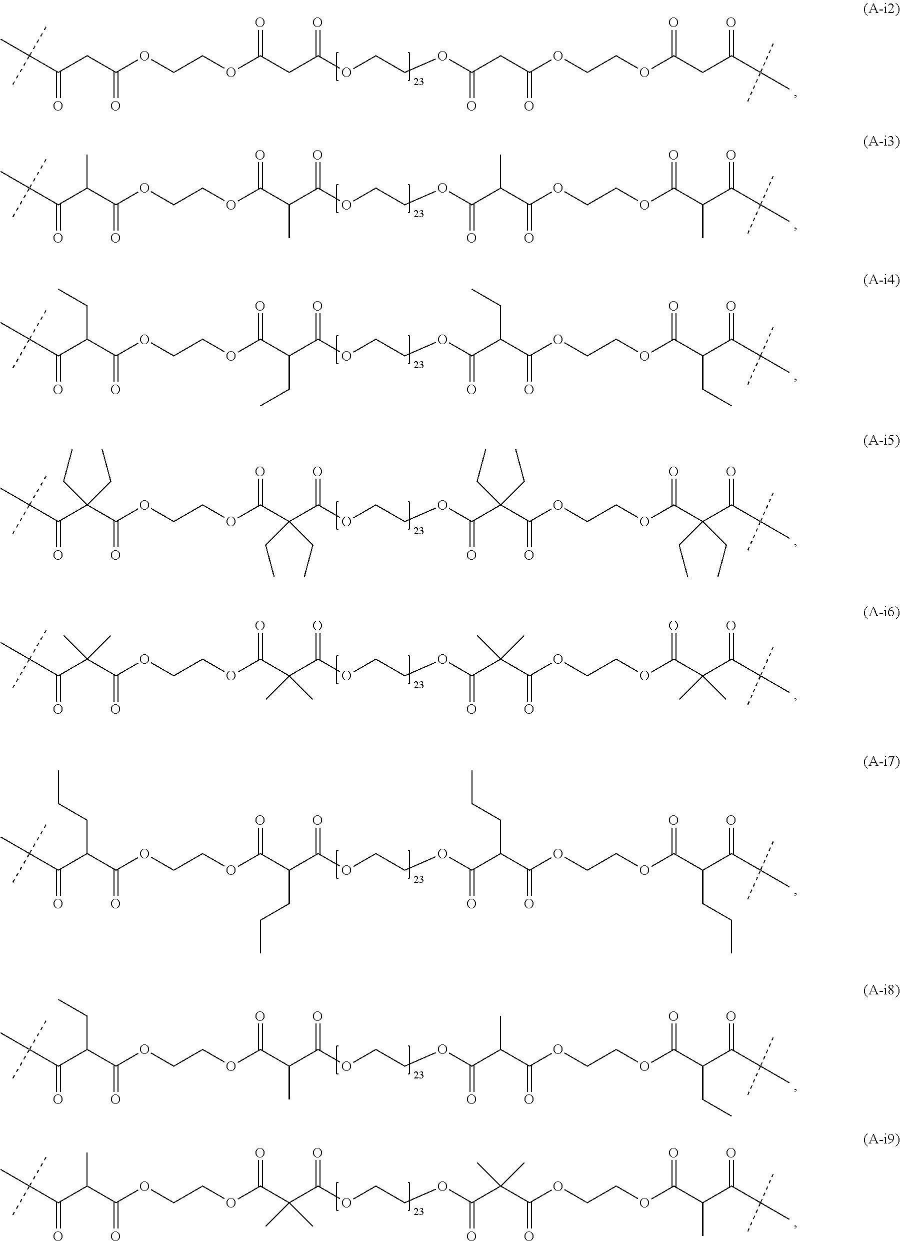

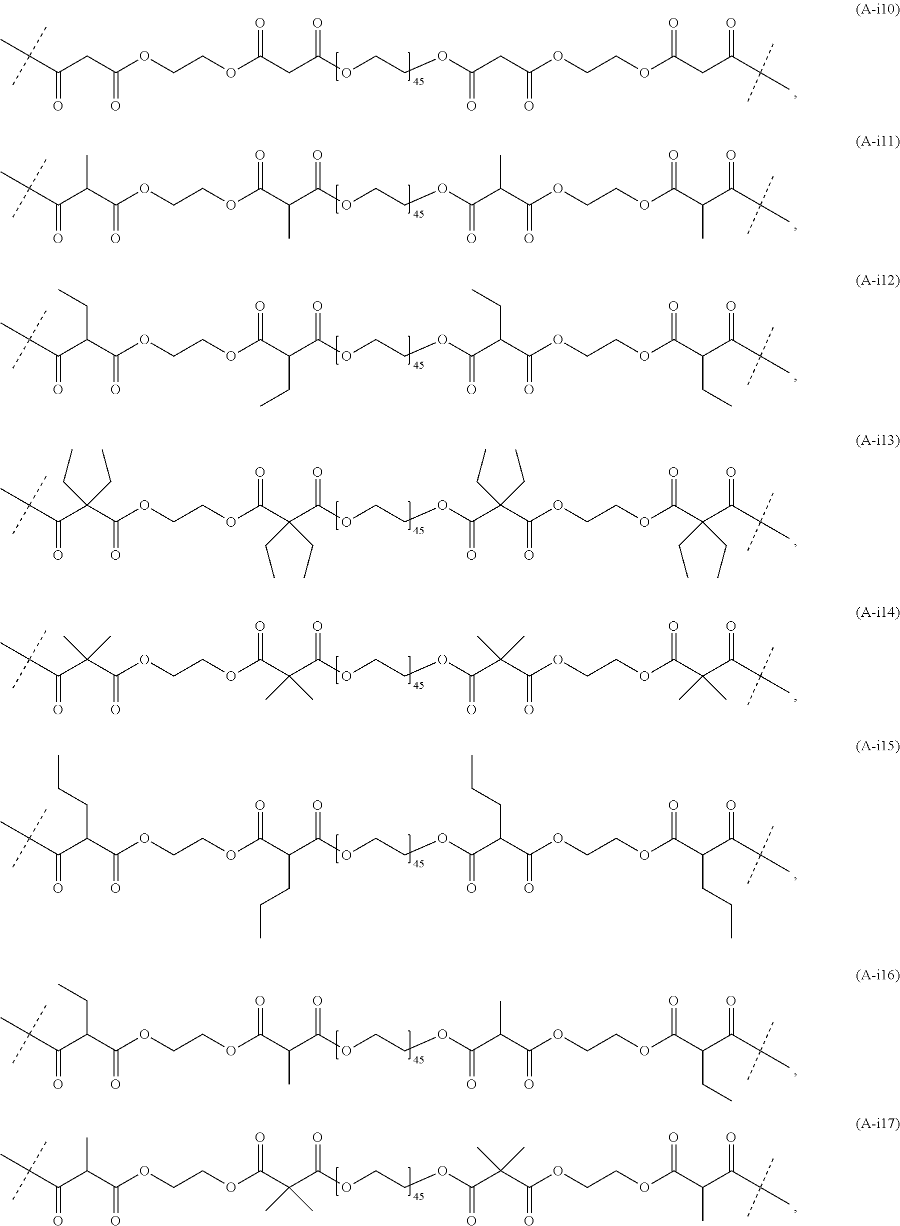

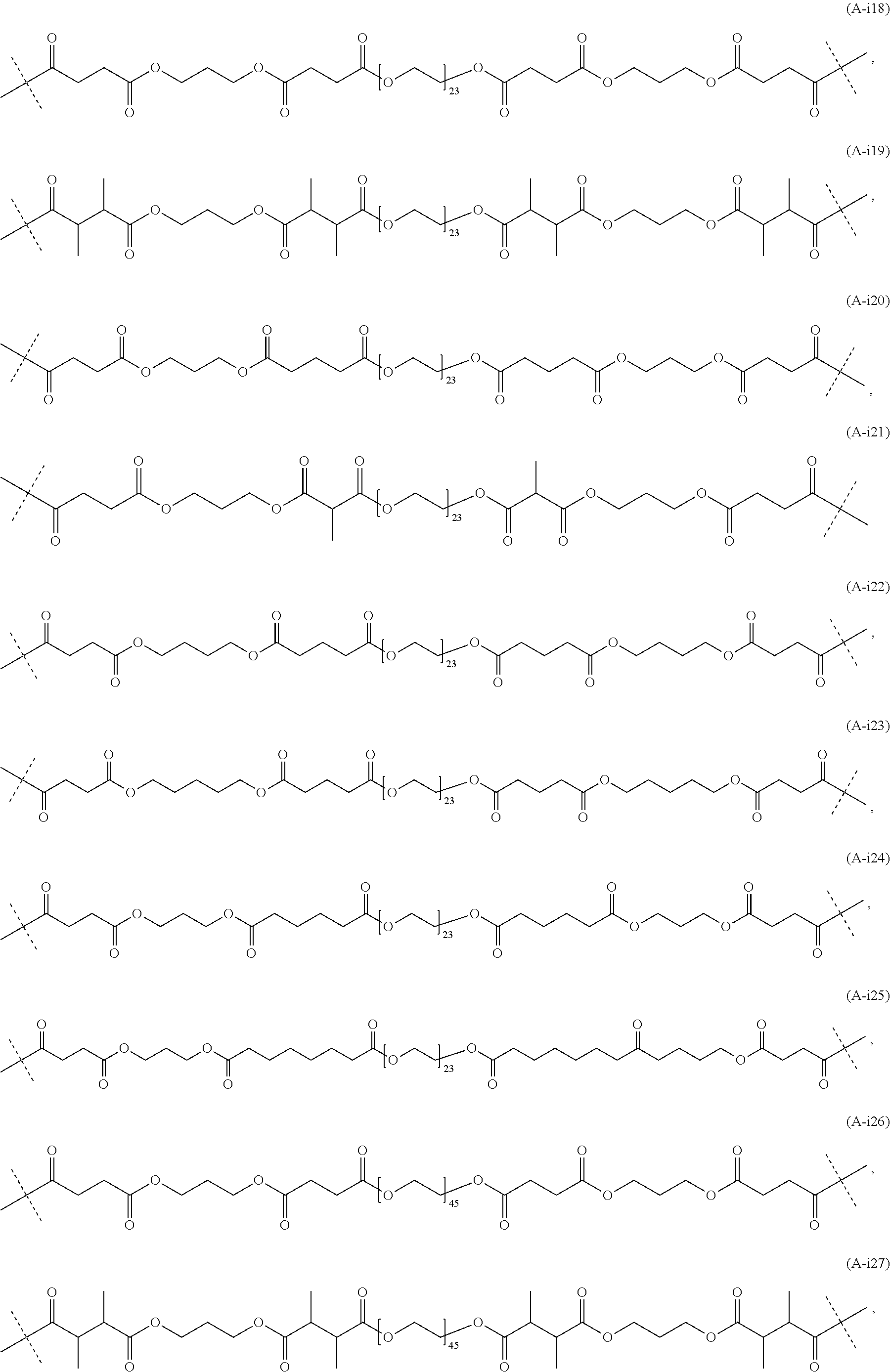

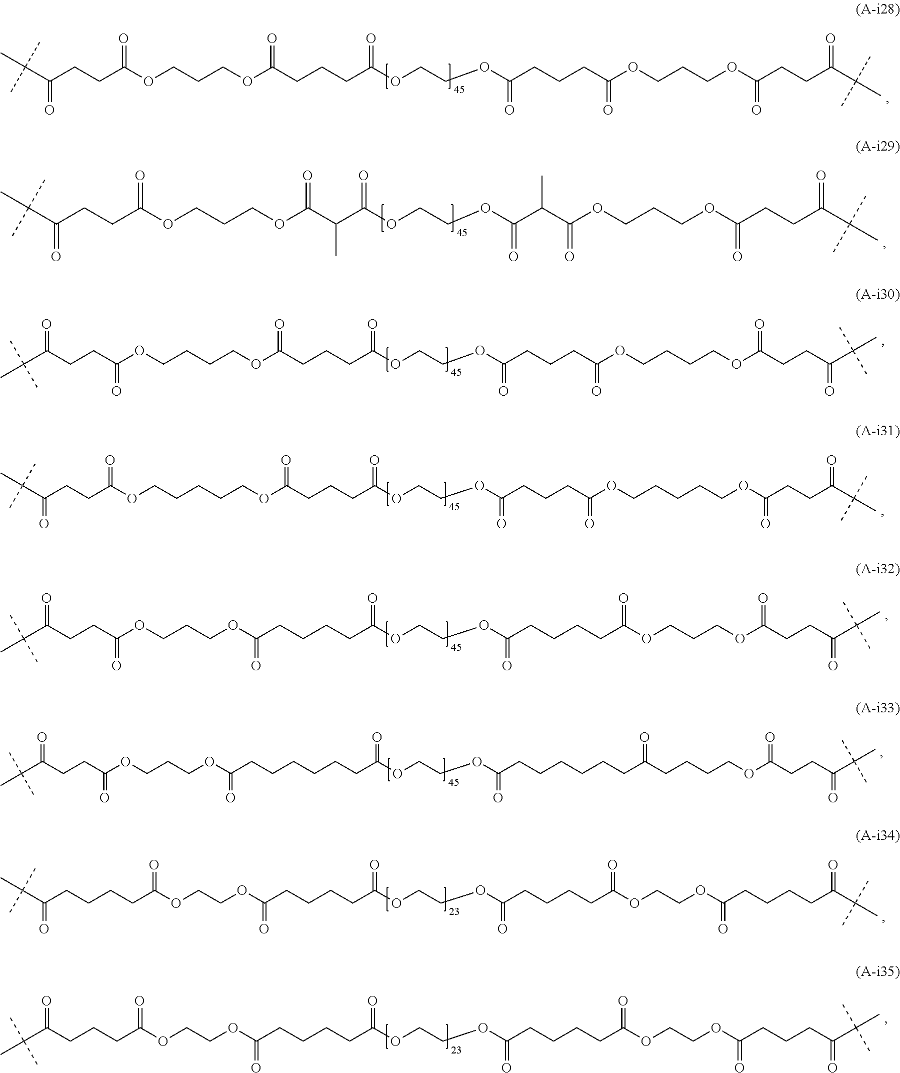

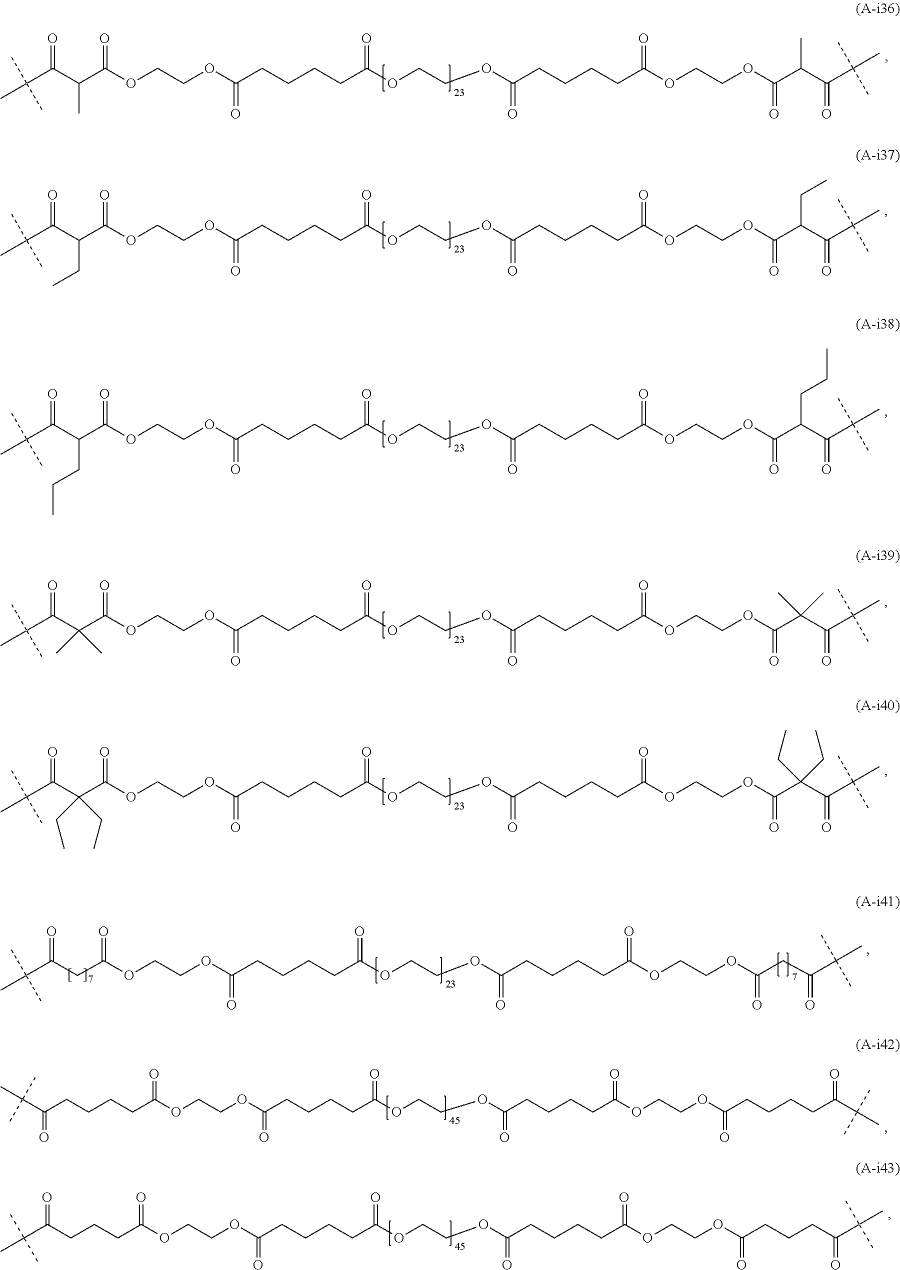

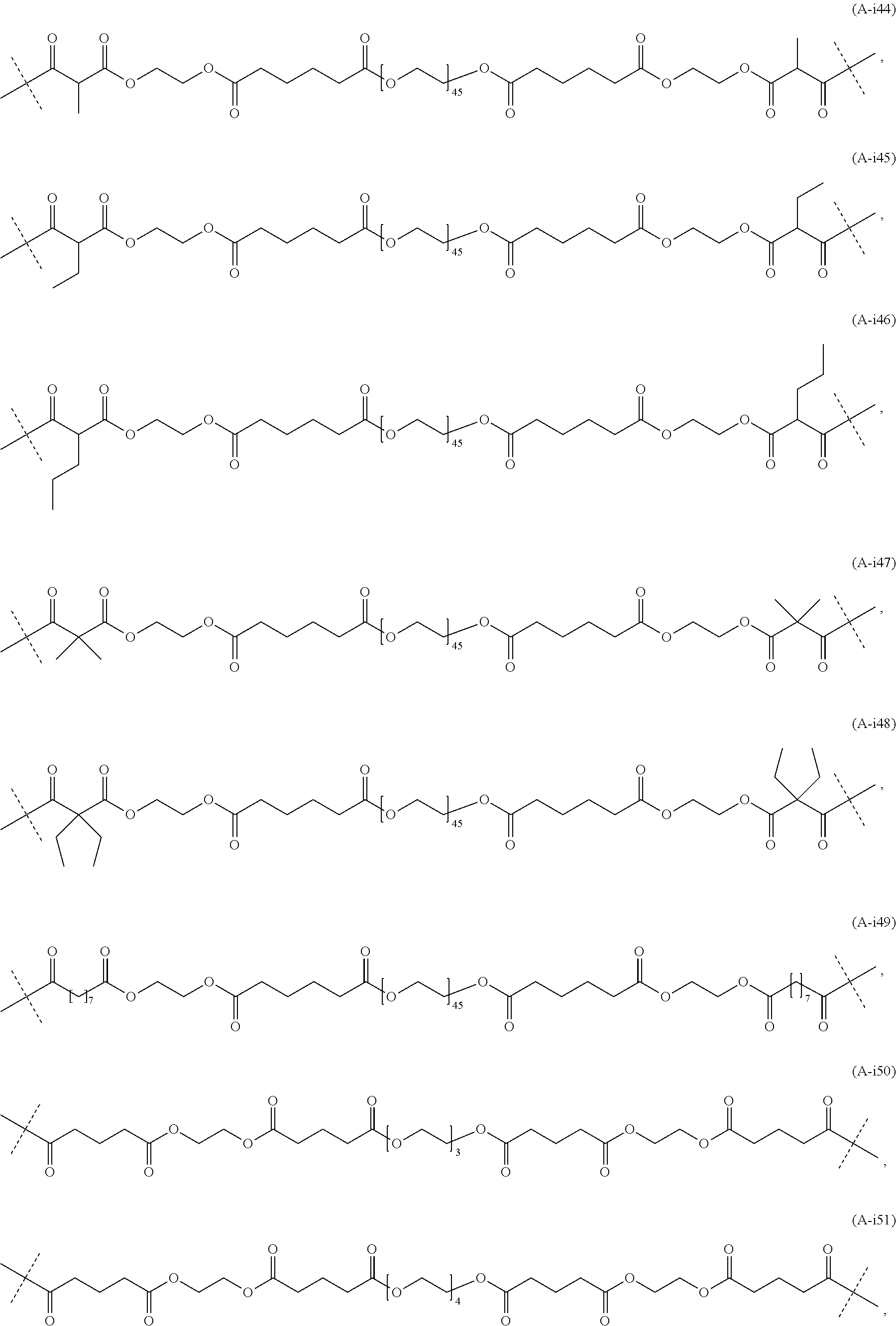

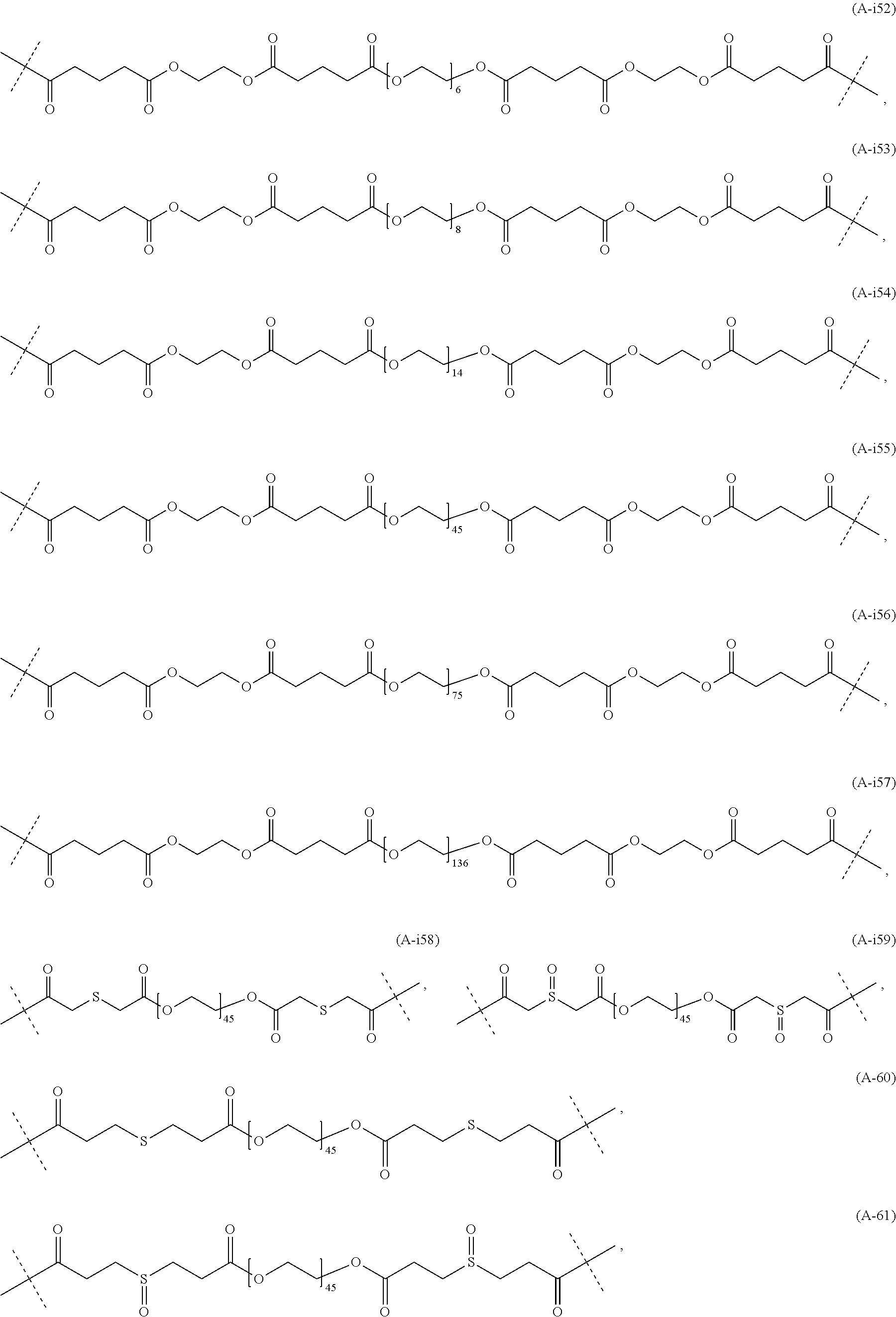

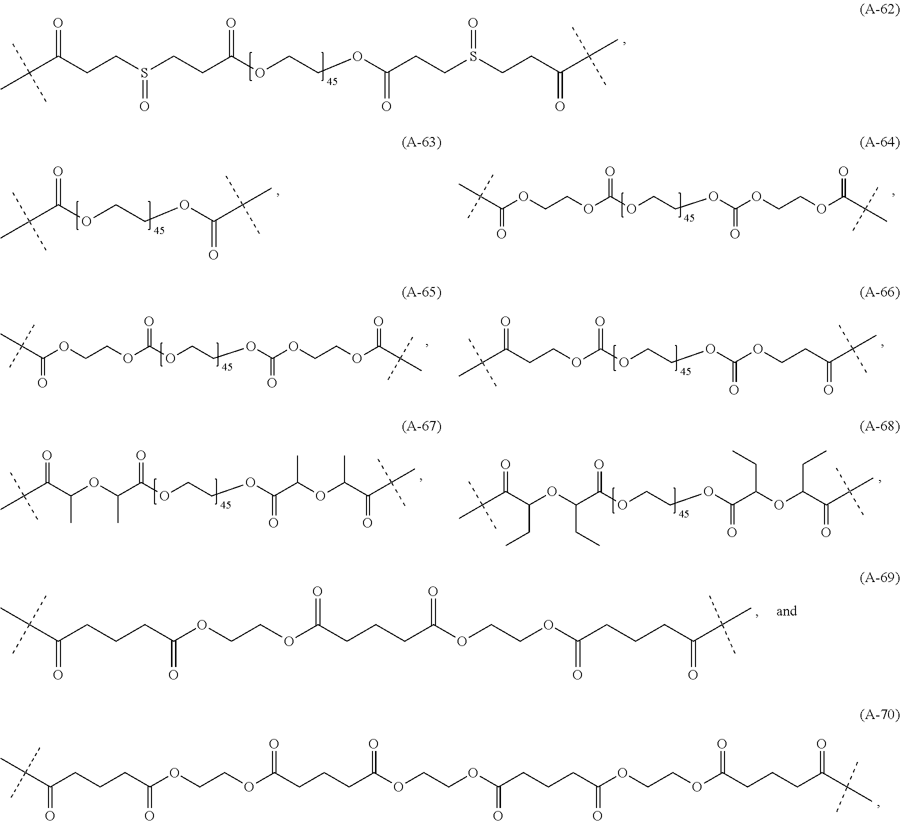

wherein
dashed lines indicate attachment to a backbone moiety or to a spacer moiety —SP—.

In certain embodiments a crosslinker moiety —CL- is of formula (A-i2). In certain embodiments a crosslinker moiety —CL- is of formula (A-i3). In certain embodiments a crosslinker moiety —CL- is of formula (A-i4). In certain embodiments a crosslinker moiety —CL- is of formula (A-i5). In certain embodiments a crosslinker moiety —CL- is of formula (A-i6). In certain embodiments a crosslinker moiety —CL- is of formula (A-i7). In certain embodiments a crosslinker moiety —CL- is of formula (A-i8). In certain embodiments a crosslinker moiety —CL- is of formula (A-i9). In certain embodiments a crosslinker moiety —CL- is of formula (A-i10). In certain embodiments a crosslinker moiety —CL- is of formula (A-i11). In certain embodiments a crosslinker moiety —CL- is of formula (A-i12). In certain embodiments a crosslinker moiety —CL- is of formula (A-i13). In certain embodiments a crosslinker moiety —CL- is of formula (A-i14). In certain embodiments a crosslinker moiety —CL- is of formula (A-i15). In certain embodiments a crosslinker moiety —CL- is of formula (A-i16). In certain embodiments a crosslinker moiety —CL- is of formula (A-i17). In certain embodiments a crosslinker moiety —CL- is of formula (A-i18). In certain embodiments a crosslinker moiety —CL- is of formula (A-i19). In certain embodiments a crosslinker moiety —CL- is of formula (A-i20). In certain embodiments a crosslinker moiety —CL- is of formula (A-i21). In certain embodiments a crosslinker moiety —CL- is of formula (A-i22). In certain embodiments a crosslinker moiety —CL- is of formula (A-i23). In certain embodiments a crosslinker moiety —CL- is of formula (A-i24). In certain embodiments a crosslinker moiety —CL- is of formula (A-i25). In certain embodiments a crosslinker moiety —CL- is of formula (A-i26). In certain embodiments a crosslinker moiety —CL- is of formula (A-i27). In certain embodiments a crosslinker moiety —CL- is of formula (A-i28). In certain embodiments a crosslinker moiety —CL- is of formula (A-i29). In certain embodiments a crosslinker moiety —CL- is of formula (A-i30). In certain embodiments a crosslinker moiety —CL- is of formula (A-i31). In certain embodiments a crosslinker moiety —CL- is of formula (A-i32). In certain embodiments a crosslinker moiety —CL- is of formula (A-i33). In certain embodiments a crosslinker moiety —CL- is of formula (A-i34). In certain embodiments a crosslinker moiety —CL- is of formula (A-i35). In certain embodiments a crosslinker moiety —CL- is of formula (A-i36). In certain embodiments a crosslinker moiety —CL- is of formula (A-i37). In certain embodiments a crosslinker moiety —CL- is of formula (A-i38). In certain embodiments a crosslinker moiety —CL- is of formula (A-i39). In certain embodiments a crosslinker moiety —CL- is of formula (A-i40). In certain embodiments a crosslinker moiety —CL- is of formula (A-i41). In certain embodiments a crosslinker moiety —CL- is of formula (A-i42). In certain embodiments a crosslinker moiety —CL- is of formula (A-i43). In certain embodiments a crosslinker moiety —CL- is of formula (A-i44). In certain embodiments a crosslinker moiety —CL- is of formula (A-i45). In certain embodiments a crosslinker moiety —CL- is of formula (A-i46). In certain embodiments a crosslinker moiety —CL- is of formula (A-i47). In certain embodiments a crosslinker moiety —CL- is of formula (A-i48). In certain embodiments a crosslinker moiety —CL- is of formula (A-i49). In certain embodiments a crosslinker moiety —CL- is of formula (A-i50). In certain embodiments a crosslinker moiety —CL- is of formula (A-i51). In certain embodiments a crosslinker moiety —CL- is of formula (A-i52). In certain embodiments a crosslinker moiety —CL- is of formula (A-i53). In certain embodiments a crosslinker moiety —CL- is of formula (A-i54). In certain embodiments a crosslinker moiety —CL- is of formula (A-i55). In certain embodiments a crosslinker moiety —CL- is of formula (A-i56). In certain embodiments a crosslinker moiety —CL- is of formula (A-i57). In certain embodiments a crosslinker moiety —CL- is of formula (A-i58). In certain embodiments a crosslinker moiety —CL- is of formula (A-i59). In certain embodiments a crosslinker moiety —CL- is of formula (A-i60). In certain embodiments a crosslinker moiety —CL- is of formula (A-i61). In certain embodiments a crosslinker moiety —CL- is of formula (A-i62). In certain embodiments a crosslinker moiety —CL- is of formula (A-i63). In certain embodiments a crosslinker moiety —CL- is of formula (A-i64). In certain embodiments a crosslinker moiety —CL- is of formula (A-i65). In certain embodiments a crosslinker moiety —CL- is of formula (A-i66). In certain embodiments a crosslinker moiety —CL- is of formula (A-i67). In certain embodiments a crosslinker moiety —CL- is of formula (A-i68). In certain embodiments a crosslinker moiety —CL- is of formula (A-i69). In certain embodiments a crosslinker moiety —CL- is of formula (A-i70).

In certain embodiments a backbone moiety has a molecular weight ranging from 1 kDa to 20 kDa, such as from 1 to 18 kDa, from 2 to 15 kDa, from 4 to 13 kDa or from 5 to 12 kDa.

In certain embodiments a backbone moiety comprises at least one polymeric moiety. In certain embodiments a backbone moiety comprises a multi-arm polymer, such as a polymer having 3 to 8 polymeric arms, such as having three polymeric arms, four polymeric arms, five polymeric arms, six polymeric arms, seven polymeric arms or eight polymeric arms. In certain embodiments a backbone moiety comprises 3 to 6 polymeric arms.

In certain embodiments such polymeric arm comprises a polymer selected from the group consisting of the group consisting of 2-methacryloyl-oxyethyl phosphoyl cholins, poly(acrylic acids), poly(acrylates), poly(acrylamides), poly(alkyloxy) polymers, poly(amides), poly(amidoamines), poly(amino acids), poly(anhydrides), poly(aspartamides), poly(butyric acids), poly(glycolic acids), polybutylene terephthalates, poly(caprolactones), poly(carbonates), poly(cyanoacrylates), poly(dimethylacrylamides), poly(esters), poly(ethylenes), poly(ethyleneglycols), poly(ethylene oxides), poly(ethyl phosphates), poly(ethyloxazolines), poly(glycolic acids), poly(hydroxyethyl acrylates), poly(hydroxyethyl-oxazolines), poly(hydroxymethacrylates), poly(hydroxypropylmethacrylamides), poly(hydroxypropyl methacrylates), poly(hydroxypropyloxazolines), poly(iminocarbonates), poly(lactic acids), poly(lactic-co-glycolic acids), poly(methacrylamides), poly(methacrylates), poly(methyloxazolines), poly(organophosphazenes), poly(ortho esters), poly(oxazolines), poly(propylene glycols), poly(siloxanes), poly(urethanes), poly(vinyl alcohols), poly(vinyl amines), poly(vinylmethylethers), poly(vinylpyrrolidones), silicones, celluloses, carbomethyl celluloses, hydroxypropyl methylcelluloses, chitins, chitosans, dextrans, dextrins, gelatins, hyaluronic acids and derivatives, functionalized hyaluronic acids, alginate, mannans, pectins, rhamnogalacturonans, starches, hydroxyalkyl starches, hydroxyethyl starches and other carbohydrate-based polymers, xylans, and copolymers thereof.

In certain embodiments such polymeric arm is a PEG-based polymer. In certain embodiments such polymeric moiety is a hyaluronic acid-based polymer.

In certain embodiments a backbone moiety is of formula (B)

B*-(A-Hyp)ₓ (B),
wherein
B* is a branching core,
A is a PEG-based polymer,
Hyp is a branched moiety,
x is an integer of from 3 to 16;
and wherein each backbone moiety is connected to one or more crosslinker moieties and to one or more moieties -L²-, which crosslinker moieties and moieties -L²- are connected to Hyp, either directly or through a spacer moiety.

In certain embodiments B* of formula (B) is selected from the group consisting of polyalcohol moieties and polyamine moieties. In certain embodiments B* of formula (B) is a polyalcohol moiety. In certain embodiments B* of formula (B) is a polyamine moiety.

In certain embodiments the polyalcohol moieties for B* of formula (B) are selected from the group consisting of a pentaerythritol moiety, tripentaerythritol moiety, hexaglycerine moiety, sucrose moiety, sorbitol moiety, fructose moiety, mannitol moiety and glucose moiety. In certain embodiments B* of formula (B) is a pentaerythritol moiety, i.e. a moiety of formula

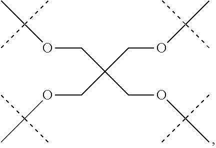

wherein dashed lines indicate attachment to -A-.

In certain embodiments the polyamine moieties for B* of formula (B) is selected from the group consisting of an ornithine moiety, diaminobutyric acid moiety, trilysine moiety, tetralysine moiety, pentalysine moiety, hexalysine moiety, heptalysine moiety, octalysine moiety, nonalysine moiety, decalysine moiety, undecalysine moiety, dodecalysine moiety, tridecalysine moiety, tetradecalysine moiety and pentadecalysine moiety. In certain embodiments B* of formula (B) is selected from the group consisting of an ornithine moiety, diaminobutyric acid moiety and a trilysine moiety.

A backbone moiety of formula (B) may consist of the same or different PEG-based moieties -A- and each moiety -A- may be chosen independently. In certain embodiments all moieties -A- present in a backbone moiety of formula (B) have the same structure. It is understood that the phrase “have the same structure” with regard to polymeric moieties, such as with regard to the PEG-based polymeric moiety -A-, means that the number of monomers of the polymer, such as the number of ethylene glycol monomers, may vary due to the polydisperse nature of polymers. In certain embodiments the number of monomer units does not vary by more than a factor of 2 between all moieties -A- of a hydrogel.

In certain embodiments each -A- of formula (B) has a molecular weight ranging from 0.3 kDa to 40 kDa; e.g. from 0.4 to 30 kDa, from 0.4 to 25 kDa, from 0.4 to 20 kDa, from 0.4 to kDa, from 0.4 to 10 kDa or from 0.4 to 5 kDa. In certain embodiments each -A- may have a molecular weight from 0.4 to 5 kDa. In certain embodiments -A- has a molecular weight of about 0.5 kDa. In certain embodiments -A- has a molecular weight of about 1 kDa. In certain embodiments -A- has a molecular weight of about 2 kDa. In certain embodiments -A- has a molecular weight of about 3 kDa. In certain embodiments -A- has a molecular weight of about 5 kDa.

In certain embodiments -A- of formula (B) is of formula (B-ia)

—(CH₂)ₙ₁(OCH₂CH₂)ₙX— (B-ia),
wherein
n1 is 1 or 2;
n is an integer ranging from 3 to 250, such as from 5 to 200, such as from 8 to 150 or from 10 to 100; and
X is a chemical bond or a linkage covalently linking A and Hyp.

In certain embodiments -A- of formula (B) is of formula (B-ib)

—(CH₂)ₙ₁(OCH₂CH₂)ₙ—(CH₂)ₙ₂X— (B-ib),
wherein
n1 is 1 or 2;
n is an integer ranging from 3 to 250, such as from 5 to 200, such as from 8 to 150 or from 10 to 100;
n2 is 0 or 1; and
X is a chemical bond or a linkage covalently linking A and Hyp.

In certain embodiments -A- of formula (B) is of formula (B-ia′)

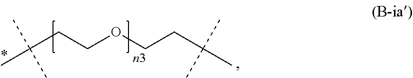

wherein
the dashed line marked with the asterisk indicates attachment to B*,
the unmarked dashed line indicates attachment to -Hyp; and
n3 is an integer ranging from 10 to 50.

In certain embodiments n3 of formula (B-ia′) is 25. In certain embodiments n3 of formula (B-ia′) is 26. In certain embodiments n3 of formula (B-ia′) is 27. In certain embodiments n3 of formula (B-ia′) is 28. In certain embodiments n3 of formula (B-ia′) is 29. In certain embodiments n3 of formula (B-ia′) is 30.

In certain embodiments a moiety B*-(A)₄ is of formula (B-ii)

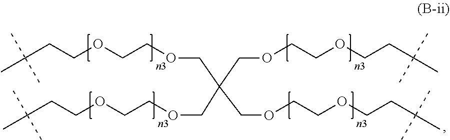

wherein
dashed lines indicate attachment to Hyp; and
each n3 is independently an integer selected from 10 to 50.

In certain embodiments n3 of formula (B-ii) is 25. In certain embodiments n3 of formula (B-a) is 26. In certain embodiments n3 of formula (B-ii) is 27. In certain embodiments n3 of formula (B-ii) is 28. In certain embodiments n3 of formula (B-ii) is 29. In certain embodiments n3 of formula (B-ii) is 30.

A backbone moiety of formula (B) may consist of the same or different dendritic moieties -Hyp and each -Hyp may be chosen independently of the others. In certain embodiments all moieties -Hyp present in a backbone moiety of formula (B) have the same structure.

In certain embodiments each -Hyp of formula (B) has a molecular weight in the range of from 0.3 kDa to 5 kDa.

In certain embodiments -Hyb is selected from the group consisting of a moiety of formula (B-iiia)

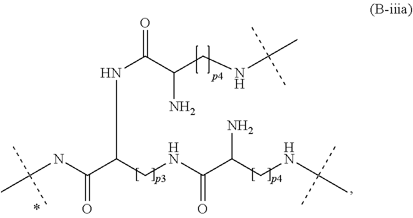

wherein
the dashed line marked with the asterisk indicates attachment to -A-,
the unmarked dashed lines indicate attachment to a spacer moiety —SP—, a crosslinker moiety —CL- or to -L²-; and
p2, p3 and p4 are identical or different and each is independently of the others an integer from 1 to 5;
   a moiety of formula (B-iiib)

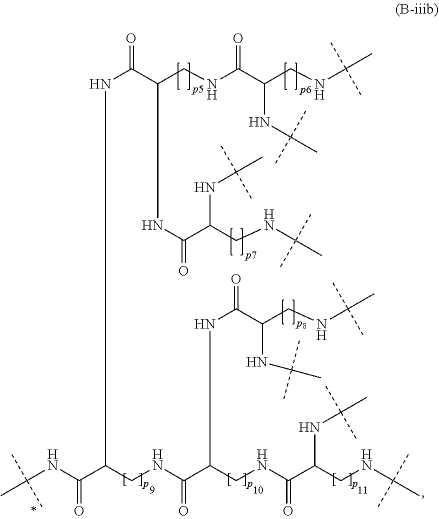

wherein
the dashed line marked with the asterisk indicates attachment to -A-,
the unmarked dashed lines indicate attachment to a spacer moiety —SP—, a crosslinker moiety —CL- or to -L²-; and
p5 to p11 are identical or different and each is independently of the others an integer from 1 to 5;
   a moiety of formula (B-iiic)

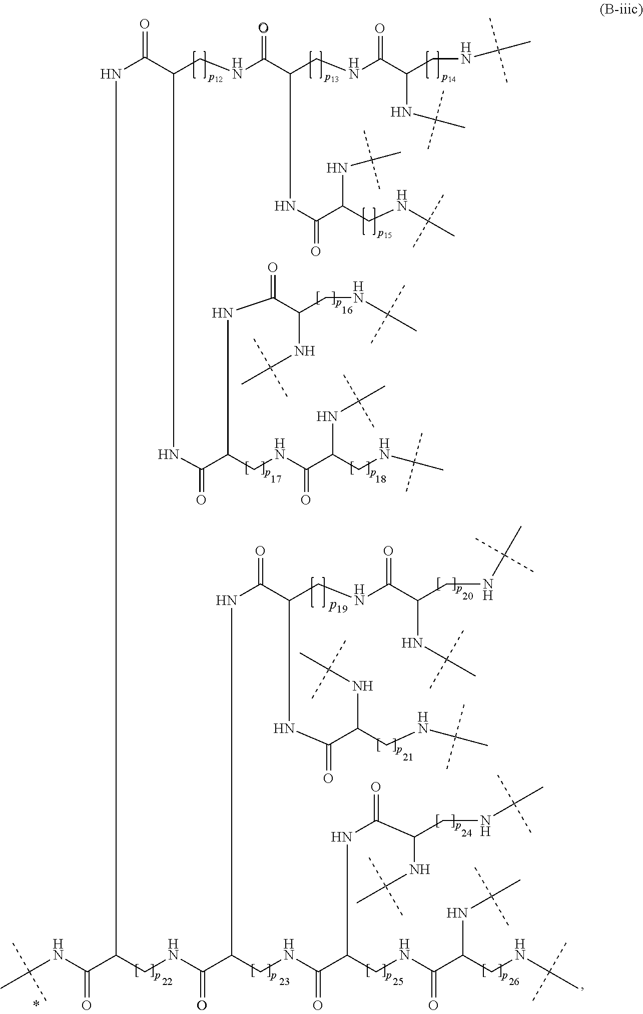

wherein
the dashed line marked with the asterisk indicates attachment to -A-, the unmarked dashed lines indicate attachment to a spacer moiety —SP—, a crosslinker moiety —CL- or to -L²-; and
p12 to p26 are identical or different and each is independently of the others an integer from 1 to 5; and
   a moiety of formula (B-iiid)

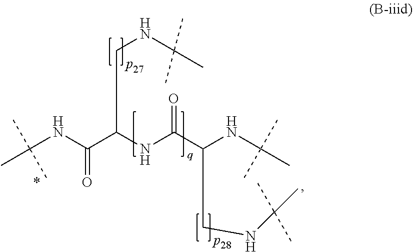

wherein
the dashed line marked with the asterisk indicates attachment to -A-,
the unmarked dashed lines indicate attachment to a spacer moiety —SP—, a crosslinker moiety —CL- or to -L²-;
p27 and p28 are identical or different and each is independently of the other an integer from 1 to 5; and
q is an integer from 1 to 8;
   wherein the moieties (B-iiia), (B-iiib), (B-iiic) and (B-iiid) may at each chiral center be in either R- or S-configuration.

In certain embodiments all chiral centers of a moiety (B-iiia), (B-iiib), (B-iiic) and (B-iiid) are in the same configuration. In certain embodiments all chiral centers of a moiety (B-iiia), (B-iiib), (B-iiic) and (B-iiid) are in R-configuration. In certain embodiments all chiral centers of a moiety (B-iiia), (B-iiib), (B-iiic) and (B-iiid) are in S-configuration.

In certain embodiments p2, p3 and p4 of formula (B-iiia) are identical. In certain embodiments p2, p3 and p4 of formula (B-iiia) are 1. In certain embodiments p2, p3 and p4 of formula (B-iiia) are 2. In certain embodiments p2, p3 and p4 of formula (B-iiia) are 3. In certain embodiments p2, p3 and p4 of formula (B-iiia) are 4. In certain embodiments p2, p3 and p4 of formula (B-iiia) are 5.

In certain embodiments p5 to p11 of formula (B-iiib) are identical. In certain embodiments p5 to p11 of formula (B-iiib) are 1. In certain embodiments p5 to p11 of formula (B-iiib) are 2.

In certain embodiments p5 to p11 of formula (B-iiib) are 3. In certain embodiments p5 to p11 of formula (B-iiib) are 4. In certain embodiments p5 to p11 of formula (B-iiib) are 5.

In certain embodiments p12 to p26 of formula (B-iiic) are identical. In certain embodiments p12 to p26 of formula (B-iiic) are 1. In certain embodiments p12 to p26 of formula (B-iiic) are 2. In certain embodiments p12 to p26 of formula (B-iiic) are 3. In certain embodiments p12 to p26 of formula (B-iiic) are 4. In certain embodiments p12 to p26 of formula (B-iiic) are 5.

In certain embodiments q of formula (B-iiid) q is 1. In certain embodiments q of formula (B-iiid) q is 2. In certain embodiments q of formula (B-iiid) q is 3. In certain embodiments q of formula (B-iiid) q is 4. In certain embodiments q of formula (B-iiid) q is 5. In certain embodiments q of formula (B-iiid) q is 6. In certain embodiments q of formula (B-iiid) q is 7. In certain embodiments q of formula (B-iiid) q is 8. In certain embodiments q of formula (B-iiid) is 2 or 6.

In certain embodiments p27 and p28 of formula (B-iiid) are identical. In certain embodiments p27 and p28 of formula (B-iiid) are 1. In certain embodiments p27 and p28 of formula (B-iiid) are 2. In certain embodiments p27 and p28 of formula (B-iiid) are 3. In certain embodiments p27 and p28 of formula (B-iiid) are 4. In certain embodiments p27 and p28 of formula (B-iiid) are 4.

In certain embodiments -Hyp of formula (B) comprises a branched polypeptide moiety.

In certain embodiments -Hyp is of formula (B-iiie)

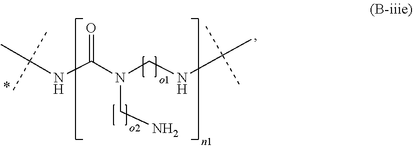

wherein
the dashed line marked with the asterisk indicates attachment to -A-,
the unmarked dashed lines indicate attachment to a spacer moiety —SP—, a crosslinker moiety —CL- or to -L²-;
o1 is an integer ranging from 1 to 10;
o2 is an integer ranging from 1 to 10; and
n1 is an integer ranging from 1 to 8.

In certain embodiments o1 of formula (B-iiie) is 2. In certain embodiments o1 of formula (B-iiie) is 3. In certain embodiments o2 of formula (B-iiie) is 2. In certain embodiments o2 of formula (B-iiie) is 3. In certain embodiments n1 of formula (B-iiie) is 2. In certain embodiments n1 of formula (B-iiie) is 3.

In certain embodiments -Hyp of formula (B) comprises a lysine moiety. In certain embodiments each -Hyp of formula (B) is independently selected from the group consisting of a trilysine moiety, tetralysine moiety, pentalysine moiety, hexalysine moiety, heptalysine moiety, octalysine moiety, nonalysine moiety, decalysine moiety, undecalysine moiety, dodecalysine moiety, tridecalysine moiety, tetradecalysine moiety, pentadecalysine moiety, hexadecalysine moiety, heptadecalysine moiety, octadecalysine moiety and nonadecalysine moiety.

In certain embodiments -Hyp comprises 3 lysine moieties. In certain embodiments -Hyb comprises 7 lysine moieties. In certain embodiments -Hyb comprises 15 lysine moieties. In certain embodiments -Hyp comprises heptalysinyl.

In certain embodiments x of formula (B) is 3. In certain embodiments x of formula (B) is 4.

In certain embodiments x of formula (B) is 4. In certain embodiments x of formula (B) is 5.

In certain embodiments x of formula (B) is 6. In certain embodiments x of formula (B) is 4.

In certain embodiments x of formula (B) is 7. In certain embodiments x of formula (B) is 8.

In certain embodiments -Hyp is of formula (B-iiif):

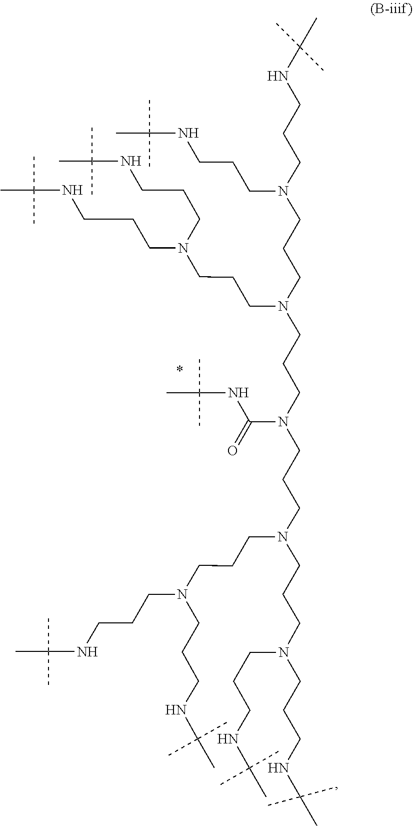

wherein
the dashed line marked with the asterisk indicates attachment to -A-,
the unmarked dashed lines indicate attachment to a spacer moiety —SP—, a crosslinker moiety —CL- or to -L²-;

In certain embodiments the backbone moiety is of formula (B-iv)

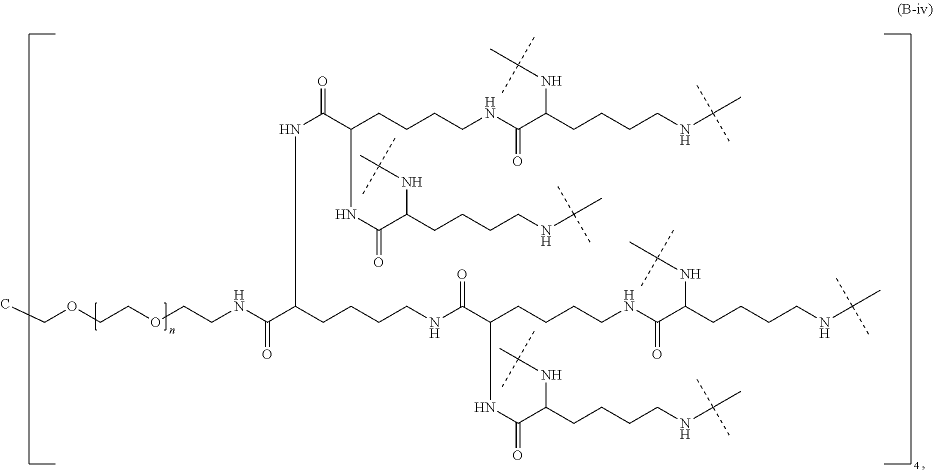

wherein
dashed lines indicate attachment to a spacer moiety —SP—, a crosslinker moiety —CL- or to -L²-; and
n ranges from 10 to 40.

In certain embodiments n of formula (B-iv) is about 28.

In certain embodiments —CL- is connected to Hyp via —SP—. In such case —SP— is defined as -L²-.

In certain embodiments there is no spacer moiety —SP— between a backbone moiety and a crosslinker moiety —CL-, i.e. —CL- is directly linked to -Hyp.

-D is a drug moiety that is covalently and reversibly conjugated to -L¹-. -D may be selected from the group consisting of peptides, proteins, oligonucleotides and small molecule drug moieties. In certain embodiments -D is a peptide drug moiety. In certain embodiments -D is a protein drug moiety. In certain embodiments -D is an oligonucleotide drug moiety. In certain embodiments -D is a small molecule drug moiety.

In certain embodiments -D is an antibiotic moiety, for example an antibiotic selected from the group consisting of aminoglycosides, tetracycline antibiotics, amphenicols, pleuromutilins, macrolid antibiotics, lincosamides, steroid antibiotics, antifolate antibiotics, sulfonamides, topoisomerase inhibitors, quinolones, fluoroquinolones, nitroimidazole antibiotics, nitrofuran antibiotics, rifamycins, glycopeptides, penicillins, cephalosporins, monobactams, beta-lactamase inhibitors, polymyxin antibiotics, lipopeptide antibiotics, oxazolidinon, antimicrobial peptides, antimicrobial proteins, porphyrins, azole antifungals, polyenes, antiprotozoal drugs, fosfomycin, cycloserine, and bacitracin.

In certain embodiments -D is an aminoglycoside, such as an aminoglycoside selected from the group consisting of streptomycin, dihydro streptomycin, neomycin, paromomycin, amikacin, kanamycin, tobramycin, spectinomycin, hygromycin b, gentamicin, plazomicin, verdamicin, netilmicin, astromicin and sisomicin. In certain embodiments -D is amikacin. In certain embodiments -D is kanamycin. In certain embodiments -D is tobramycin. In certain embodiments -D is gentamicin. In another embodiment -D is plazomicin.

In certain embodiments -D is a tetracycline antibiotic, such as a tetracycline antibiotic selected from the group consisting of doxycycline, chloretetracycline, tetracycline, metacycline, minocycline, oxytetracycline and glycocyclines, such as a glycocyclines selected from the group consisting of tigecycline, omadacycline and sarecycline. In certain embodiments -D tetracycline. In certain embodiments -D is minocycline. In certain embodiments -D is oxytetracycline. In certain embodiments -D is tigecycline. In certain embodiments -D is omadacycline. In another embodiment -D is sarecycline.

In certain embodiments -D is an amphenicol, such as an amphenicol selected from the group consisting of chloramphenicol, thiamphenicol, azidamfenicol and florfenicol.

In certain embodiments -D is a pleuromutilin, such as a pleuromutilin selected from the group consisting of azamulin, lefamulin, tiamulin and valnemulin.

In certain embodiments -D is a macrolid antibiotic, such as a macrolid antibiotic selected from the group consisting of azithromycin, boromycin, clarithromycin, oleandomycin, erythromycin, roxithromycin, spiramycin, telithromycin and tylosine.

In certain embodiments -D is a lincosamide, such as a lincosamide selected from the group consisting of clindamycin and lincomycin. In certain embodiments -D is clindamycin.

In certain embodiments -D is a steroid antibiotic, such as fusidic acid.

In certain embodiments -D is an antifolate antibiotic, such as an antifolate antibiotic selected from the group consisting of trimethoprim and iclaprim.

In certain embodiments -D is a sulfonamide, such as a sulfonamide selected from the group consisting of sufathiazole, sulfamethoxazole, sulfadiazine and sulfamerazine.

In certain embodiments -D is a topoisomerase inhibitor, such as a topoisomerase inhibitor selected from the group consisting of flumequine, nalidixic acid, oxolinic acid and pipemidic acid. In certain embodiments -D is nalidixic acid.

In certain embodiments -D is a quinolone or fluroquinolone, such as a quinolone or fluroquinolone selected from the group consisting of nemonoxacin, ciprofloxacin, ofloxacin, norfloxacin, pefloxacin, levofloxacin, sparfloxacin, moxifloxacin, gatifloxacin, difloxacin, enrofloxacin, marbofloxacin, delafloxacin and nemonovobiocin. In certain embodiments -D is ciprofloxacin. In certain embodiments -D is levofloxacin. In certain embodiments -D is delafloxacin.

In certain embodiments -D is a nitroimidazole antibiotic, such as metronidazole.

In certain embodiments -D is a nitrofuran antibiotic, such as a nitrofuran antibiotic selected from the group consisting of nitrofurantoin and furazolidone.

In certain embodiments -D is a rifamycin, such as rifampicin.

In certain embodiments -D is a glycopeptide, such as a glycoprotein selected from the group consisting of vancomycin, oritavancin, telavancin, dalbavancin and teicoplanin. In certain embodiments -D is vancomycin. In certain embodiments -D is oritavancin. In certain embodiments -D is telavancin. In another embodiment -D is dalbavancin.

In certain embodiments -D is a penicillin, such as a penicillin selected from the group consisting of penams, penems and carbapenems. In certain embodiments such penams are selected from the group consisting of amoxicillin, ampicillin, carbenicillin, ticarcillin, temocillin, aziocillin, piperacillin, mezlocillin, mecillinam, benzylpenicillin, cloxacillin, dicloxacillin, flucloxacillin, oxacillin, methicillin and nafcillin. In certain embodiments such penems and carbapenes are selected from the group consisting of faropenem, ertapenem, doripenem, thiopenem, sulopenem, imipenem and meropenem. In certain embodiments -D is imipenem. In another embodiment -D is meropenem.

In certain embodiments -D is a cephalosporin, such as a cephalosporin selected from the group consisting of cefazolin, cefadroxil, cefalexin, cefradine, cefaclor, cefamandole, cefminox, cefotiam, cefjprozil, cefuroxime, cefoxitin, cefotetan, cefmetazole, cefixime, ceftriaxone, ceftazidime, cefoperazone, cefjpodoxime, cefdinir, cefditoren, cefotaxime, cefsulodin, cefteram, ceftibuten, ceftizoxime, cefepime, cefozopran, cefpirome, ceftaroline and ceftobiprole. In certain embodiments -D is cefazolin. In certain embodiments -D is cephalexin. In certain embodiments -D is ceftaroline. In certain embodiments -D is ceftobiprole. Cepholosporins are also known as cephamycins.

In certain embodiments -D is a monobactam, such as aztreonam.

In certain embodiments -D is a beta-lactamase inhibitor, such as a beta-lactamase inhibitor selected from the group consisting of sulbactam, tazobactam, clavulanic acid and cefdinir.

In certain embodiments -D is a polymycin antibiotic, such as a polymcin antibiotic selected from the group consisting of colistin and polymyxin B. In certain embodiments -D is colistin. In certain embodiments -D is polymyxin B.

In certain embodiments -D is a lipopeptide antibiotic, such as a lipopeptide antibiotic selected from the group consisting of daptomycin, arylomycins and gramicidin. In certain embodiments -D is daptomycin. Daptomycin has the following chemical structure

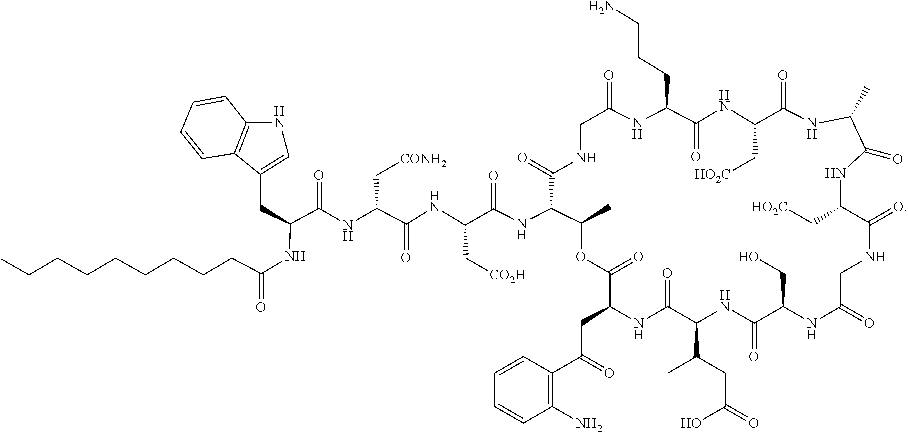 [SMILES: CCCCCCCCCC(=O)N[C@@H](Cc1c[nH]c2ccccc12)C(=O)N[C@H](CC(N)=O)C(=O)N[C@@H](CC(=O)O)C(=O)N[C@@H]1C(=O)NCC(=O)N[C@@H](CCCN)C(=O)N[C@@H](CC(=O)O)C(=O)N[C@H](C)C(=O)N[C@@H](CC(=O)O)C(=O)NCC(=O)N[C@H](CO)C(=O)N[C@@H](C(C)CC(=O)O)C(=O)N[C@@H](CC(=O)c2ccccc2N)C(=O)O[C@@H]1C]

In certain embodiments -D is an oxazolidinon, such as an oxazolidinon selected from the group consisting of linezolid, tedizolid, esperezolid, posizolid, radezolid, sutezolid and cadazolid. In certain embodiments -D is tedizolid.

In certain embodiments -D is an antimicrobial peptide, such as an antimicrobial peptide selected from the group consisting of cationic amphipathic peptides (CAP) and host defense proteins (HDP). In certain embodiments such CAP is selected from the group consisting of omiganan pentahydrochloride and novispirin g-10. In certain embodiments such HDP is brilacidin.

In certain embodiments -D is an antimicrobial protein, such as lysins.

In certain embodiments -D is a porphyrin, such as exeporfinium chloride.

In certain embodiments -D is an azole antifungal, such as an azole antifungal selected from the group consisting of fluconazole, isavuconazonium sulfate, posaconazole, itraconazole, voriconazole, albaconazole and miconazole. In certain embodiments -D is fluconazole. In certain embodiments -D is voriconazole. In certain embodiments -D is albaconazole.

In certain embodiments -D is a polyene, such as a polyene selected from the group consisting of amphotericin, echinocandins, flucytosine, tavaborole and triterpinoids. In certain embodiments an echinocandin is selected from the group consisting of caspofungin, micafungin, anidulafungin, cilofungin and rezafungin. In certain embodiments -D is amphotericin. In certain embodiments -D is caspofungin. In certain embodiments -D is micafungin. In certain embodiments -D is anidulafungin. In certain embodiments -D is cilofungin. In certain embodiments -D is rezafungin.

In certain embodiments -D is an antiprotozoal drug moiety, such as an antiprotozoal drug moiety selected from the list comprising eflomithine, furazolidone, melarsoprol, nifursemizone, omidazole, pentamidine, pyrimethamine, quinapyramine, tinidazole, chlorproguanil, proguanil, atovaquone, dehydro emetine, diloxanide, eflomithine, halofantrine, lumefantrine, mepacrine, miltefosine, nitazoxanide, tizoxanide, pyronaridine, suramin, amodiaquine, chloroquine, hydroxychloroquine, primaquine, pamaquine, tafenoquine, mefloquine, artemether, artemisinin, artemotil, artesunate and dihydroartemisinin.

It was surprisingly found that when —Z is a hydrogel, such hydrogel provides a protective environment for the antibiotic moieties that prevents their hydrolysis. This effect is particularly useful for antibiotic moieties comprising for example a lactone moiety, such as daptomycin, erythromycin, clarithromycin, azithromycin, boromycin, oleandomycin, roxithromycin, spiramycin, telithromycin, arylomycins, tylosine and linezolid, because lactone hydrolysis tends to lead to a loss of activity which reduces overall treatment efficacy.

In certain embodiments all moieties -D of a conjugate are identical. In another embodiment the conjugate comprises more than one type of -D, i.e. two or more different types of -D, such as two different types of -D, three different types of -D, four different types of -D or five different types of -D. If the conjugate comprises more than one type of -D one preferred combination is a combination of a beta-lactamase inhibitor and an antibiotic selected from the group consisting of penicillins, cephalosporins and monobactam antibiotics. Accordingly, in certain embodiments the conjugates of the present invention may comprise a beta-lactamase inhibitor and a penicillin. In certain embodiments the conjugates of the present invention may comprise a beta-lactamase inhibitor and a cephalosporin. In certain embodiments the conjugates of the present invention may comprise a beta-lactamase inhibitor and a monobactam antibiotic.

In certain embodiments -D is a pattern recognition receptor agonist (“PRRA”). Such PRRA may for example be selected from the group consisting of Toll-like receptor (TLR) agonists, NOD-like receptors (NLRs), RIG-I-like receptors, cytosolic DNA sensors, STING, and aryl hydrocarbon receptors (AhR).

In certain embodiments -D is a Toll-like receptor agonist. In certain embodiments -D is a NOD-like receptor. In certain embodiments -D is a RIG-I-like receptor. In certain embodiments -D is a cytosolic DNA sensor. In certain embodiments -D is a STING. In certain embodiments -D is an aryl hydrocarbon receptor.

If -D is a Toll-like receptor agonist, such Toll-like receptor agonists may be selected from the group consisting of agonists of TLR1/2, such as peptidoglycans, lipoproteins, Pam3CSK4, Amplivant, SLP-AMPLIVANT, HESPECTA, ISA101 and ISA201; agonists of TLR2, such as LAM-MS, LPS-PG, LTA-BS, LTA-SA, PGN-BS, PGN-EB, PGN-EK, PGN-SA, CL429, FSL-1, Pam2CSK4, Pam3CSK4, zymosan, CBLB612, SV-283, ISA204, SMP105, heat killed Listeria monocytogenes; agonists of TLR3, such as poly(A:U), poly(I:C) (poly-ICLC), rintatolimod, apoxxim, IPH3102, poly-ICR, PRV300, RGCL2, RGIC.1, Riboxxim (RGC100, RGIC100), Riboxxol (RGIC50) and Riboxxon; agonists of TLR4, such as lipopolysaccharides (LPS), neoceptin-3, glucopyranosyl lipid adjuvant (GLA), GLA-SE, G100, GLA-AF, clinical center reference endotoxin (CCRE), monophosphoryl lipid A, grass MATA MPL, PEPA10, ONT-10 (PET-Lipid A, oncothyreon), G-305, ALD046, CRX527, CRX675 (RC527, RC590), GSK1795091, OM197MPAC, OM294DP and SAR439794; agonists of TLR2/4, such as lipid A, OM174 and PGN007; agonists of TLR5, such as flagellin, entolimod, mobilan, protectan CBLB501; agonists of TLR6/2, such as diacylated lipoproteins, diacylated lipopeptides, FSL-1, MALP-2 and CBLB613; agonists of TLR7, such as CL264, CL307, imiquimod (R837), TMX-101, TMX-201, TMX-202, TMX302, gardiquimod, S-27609, 851, UC-IV150, 852A (3M-001, PF-04878691), loxoribine, polyuridylic acid, GSK2245035, GS-9620, RO6864018 (ANA773, RG7795), R07020531, isatoribine, AN0331, ANA245, ANA971, ANA975, DSP0509, DSP3025 (AZD8848), GS986, MBS2, MBS5, RG7863 (RO6870868), sotirimod, SZU101 and TQA3334; agonists of TLR8, such as ssPolyUridine, ssRNA40, TL8-506, XG-1-236, VTX-2337 (motolimod), VTX-1463, VTX378, VTX763, DN1508052 and GS9688; agonists of TLR7/8, such as CL075, CL097, poly(dT), resiquimod (R-848, VML600, S28463), MEDI9197 (3M-052), NKTR262, DV1001, IM04200, IPH3201 and VTX1463; agonists of TLR9, such as CpG DNA, CpG ODN, lefitolimod (MGN1703), SD-101, QbG10, CYT003, CYT003-QbG10, DUK-CpG-001, CpG-7909 (PF-3512676), GNKG168, EMD 1201081, IMO-2125, IMO-2055, CpG10104, AZD1419, AST008, IM02134, MGN1706, IRS 954, 1018 ISS, actilon (CPG10101), ATP00001, AVE0675, AVE7279, CMP001, DIMS0001, DIMS9022, DIMS9054, DIMS9059, DV230, DV281, EnanDIM, heplisav (V270), kappaproct (DIMS0150), NJP834, NPI503, SAR21609 and tolamba; and agonists of TLR7/9, such as DV1179.

In certain embodiments -D is an agonist of TLR1/2. In certain embodiments -D is an agonist of TLR2. In certain embodiments -D is an agonist of TLR3. In certain embodiments -D is an agonist of TLR4. In certain embodiments -D is an agonist of TLR2/4. In certain embodiments -D is an agonist of TLR5. In certain embodiment -D is an agonist of TLR6/2. In certain embodiments -D is an agonist of TLR7. In certain embodiments -D is an agonist of TLR8. In certain embodiments -D is an agonist of TLR7/8. In certain embodiments -D is an agonist of TLR9.

Examples for CpG ODN are ODN 1585, ODN 2216, ODN 2336, ODN 1668, ODN 1826, ODN 2006, ODN 2007, ODN BW006, ODN D-SL01, ODN 2395, ODN M362 and ODN D-SL03.

In certain embodiments at least some moieties -D of the conjugate are imiquimod, such as about 10%, about 20%, about 30%, about 40%, about 50%, about 60%, about 70%, about 80%, about 90% or 100% of all moieties -D present in the conjugate. In certain embodiments at least some moieties -D of the conjugate are resiquimod, such as about 10%, about 20%, about 30%, about 40%, about 50%, about 60%, about 70%, about 80%, about 90% or 100% of all moieties -D present in the conjugate. In certain embodiments at least some moieties -D of the conjugate are SD-101, such as about 10%, about 20%, about 30%, about 40%, about 50%, about 60%, about 70%, about 80%, about 90% or 100% of all moieties -D present in the conjugate. In certain embodiments at least some moieties -D of the conjugate are CMP001, such as about 10%, about 20%, about 30%, about 40%, about 50%, about 60%, about 70%, about 80%, about 90% or 100% of all moieties -D present in the conjugate.

If -D is a NOD-like receptor, such NOD-like receptor may be selected from the group consisting of agonists of NOD1, such as C12-iE-DAP, C14-Tri-LAN-Gly, iE-DAP, iE-Lys, and Tri-DAP; and agonists of NOD2, such as L18-MDP, MDP, M-TriLYS, murabutide and N-glycolyl-MDP.

In certain embodiments -D is an agonist of NOD1. In certain embodiments -D is an agonist of NOD2.

If -D is a RIG-I-like receptor, such RIG-I-like receptor may be selected from the group consisting of 3p-hpRNA, 5′ppp-dsRNA, 5′ppp RNA (M8), 5′OH RNA with kink (CBS-13-BPS), 5′PPP SLR, KIN 100, KIN 101, KIN1000, KIN1400, KIN1408, KIN1409, KIN1148, KIN131A, poly(dA:dT), SB9200, RGT100 and hiltonol.

If -D is a cytosolic DNA sensor, such cytosolic DNA sensor may be selected from the group consisting of cGAS agonists, dsDNA-EC, G3-YSD, HSV-60, ISD, ODN TTAGGG (A151), poly(dG:dC) and VACV-70.

If -D is a STING, such STING may be selected from the group consisting of MK-1454, ADU-S100 (MIW815), 2′3′-cGAMP, 3′3′-cGAMP, c-di-AMP, c-di-GMP, cAIMP (CL592), cAIMP difluor (CL614), cAIM(PS)2 difluor (Rp/Sp) (CL656), 2′2′-cGAMP, 2′3′-cGAM(PS)2 (Rp/Sp), 3′3′-cGAM fluorinated, c-di-AMP fluorinated, 2′3′-c-di-AMP, 2′3′-c-di-AM(PS)2 (Rp,Rp), c-di-GMP fluorinated, 2′3′-c-di-GMP, c-di-IMP, c-di-UMP and DMXAA (vadimezan, ASA404).

In certain embodiments -D is MK-1454. In certain embodiments -D is ADU-S100 (MIW815). In certain embodiments -D is 2′3′-cGAMP.

If -D is an aryl hydrocarbon receptor (AhR), such AhR may be selected from the group consisting of FICZ, ITE and L-kynurenine.

In certain embodiments -D is a tyrosine kinase inhibitor (TKI).

In certain embodiments -D is selected from the group consisting of receptor tyrosine kinase inhibitors, intracellular kinase inhibitors, cyclin dependent kinase inhibitors, phosphoinositide-3-kinase (PI3K) inhibitors, mitogen-activated protein kinase inhibitors, inhibitors of nuclear factor kappa-β kinase (IKK), and Wee-1 inhibitors.

In certain embodiments -D is a receptor tyrosine kinase inhibitor. Examples for such receptor tyrosine kinase inhibitors are EGF receptor inhibitors, VEGF receptor inhibitors, C-KIT Receptor inhibitors, ERBB2 (HER2) inhibitors, ERBB3 receptor inhibitors, FGF receptor inhibitors, AXL receptor inhibitors and MET receptor inhibitors.

In certain embodiments -D is an EGF receptor inhibitor, such as afatinib, cetuximab, erlotinib, gefitinib, pertuzumab and margetuximab.

In certain embodiments -D is a VEGF receptor inhibitor, such as axitinib, lenvatinib, pegaptanib and linifanib (ABT-869). In certain embodiments -D is axitinib. In certain embodiments -D is lenvatinib.

In certain embodiments -D is a C-KIT Receptor inhibitor such as CDX0158 (KTN0158).

In certain embodiments -D is an ERBB2 (FIER2) inhibitor, such as herceptin (trastuzumab).

In certain embodiments -D is an ERBB3 receptor inhibitor, such as CDX3379 (MEDI3379, KTN3379) and AZD8931 (sapitinib).

In certain embodiments -D is an FGF receptor inhibitor such as erdafitinib.

In certain embodiments -D is an AXL receptor inhibitor such as BGB324 (BGB 324, R 428, R428, bemcentinib) and SLC391. In certain embodiments -D is a MET receptor inhibitor, such as CGEN241 or tivantinib. In certain embodiments -D is tivantinib.

In certain embodiments -D is an intracellular kinase inhibitor. Examples for such intracellular kinase inhibitors are Bruton's tyrosine kinase (BTK) inhibitors, spleen tyrosine kinase inhibitors, Bcr-Abl tyrosine kinase inhibitors, Janus kinase inhibitors and multi-specific tyrosine kinase inhibitors.

In certain embodiments -D is a BTK inhibitor, such as ibrutinib, acalabrutinib, GS-4059, spebrutinib, BGB-3111, HM71224, zanubrutinib, ARQ531, BI-BTK1 and vecabrutinib.

In certain embodiments -D is a spleen tyrosine kinase inhibitor, such as fostamatinib.

In certain embodiments -D is a Bcr-Abl tyrosine kinase inhibitor, such as imatinib and nilotinib.

In certain embodiments -D is a Janus kinase inhibitor, such as ruxolitinib, tofacitinib and fedratinib.

In certain embodiments -D is a multi-specific tyrosine kinase inhibitor, such as bosutinib, crizotinib, cabozantinib, dasatinib, entrectinib, lapatinib, mubritinib, pazopanib, sorafenib, sunitinib, SU6656 and vandetanib. In certain embodiments -D is crizotinib. In certain embodiments -D is cabozantinib which is an inhibitor of c-Met, VEGFR2, AXL and RET.

In certain embodiments -D is a cyclin dependent kinase inhibitor. Examples for cyclin dependent kinase inhibitors are copanlisib, ribociclib, palbociclib, abemaciclib, trilaciclib, purvalanol A, olomucine II and MK-7965. In certain embodiments -D is copanlisib.

In certain embodiments -D is a phophoinositide-3-kinase inhibitor. Examples for phophoinositide-3-kinase inhibitors are IPI549, GDc-0326, pictilisib, serabelisib, IC-87114, AMG319, seletalisib, idealisib and CUDC907.

In certain embodiments -D is a mitogen-activated protein kinase inhibitor. Examples for mitogen-activated protein kinase inhibitors are Ras/famesyl transferase inhibitors, Raf inhibitors, MEK inhibitors and ERK inhibitors.

In certain embodiments -D is a Ras/famesyl transferase inhibitor, such as tipirafinib and LB42708.

In certain embodiments -D is a Raf inhibitor, such as regorafenib, encorafenib, vemurafenib, dabrafenib, sorafenib, PLX-4720, GDC-0879, AZ628, lifirafenib, PLX7904 and R05126766.

In certain embodiments -D is a MEK inhibitor, such as cobimetinib, trametinib, binimetinib, selumetinib, pimasertib, refametinib and PD0325901. In certain embodiments -D or drug is cobimetinib.

In certain embodiments -D is an ERK inhibitor, such as MK-8353, GDC-0994, ulixertinib and SCH772984.

In certain embodiments -D is an inhibitors of nuclear factor IKK. Examples for inhibitors of nuclear factor kappa-β kinase (IKK) are BPI-003 and AS602868.

In certain embodiments -D is a Wee-1 inhibitor. An example of a Wee-1 inhibitor is adavosertib.

In certain embodiments -D is selected from the group consisting of lenvatinib, axitinib, cobimetinib, crizotinib, tivantinib, copanlisib and cabozantinib.

In certain embodiments -D is an anti-CTLA4 moiety.

In certain embodiments -D is selected from the group consisting of wild-type F_{c} anti-CTLA4 antibodies, Fc enhanced for effector function/FcγR binding anti-CTLA4 antibodies, anti-CTLA4 antibodies conditionally active in tumor microenvironment, anti-CTLA4 small molecules, CTLA4 antagonist fusion proteins, anti-CTLA4 anticalins, anti-CTLA4 nanobodies and anti-CTLA4 multispecific biologies based on antibodies, scFVs or other formats. In certain embodiments -D is a wild-type F_{c} anti-CTFA4 antibody. In certain embodiments -D is a Fc enhanced for effector function/FcγR binding anti-CTFA4 antibody. In certain embodiments -D is an anti-CTFA4 antibodies conditionally active in tumor microenvironment. In certain embodiments -D is an anti-CTFA4 small molecule. In certain embodiments -D is a CTLA4 antagonist fusion protein. In certain embodiments -D is an anti-CTLA4 anticalin. In certain embodiments -D is an anti-CTLA4 nanobody. In certain embodiments -D is an anti-CTLA4 multispecific biologic based on an antibody, scFV or other format. In certain embodiments -D is an anti-CTLA4 multispecific biologic based on an antibody. In certain embodiments -D is an anti-CTLA4 multispecific based on a scFV.

Exemplary wild-type Fc anti-CTLA4 antibody are selected from the group consisting of ipilimumab, tremelimumab, MK-1308, CBT509 (also known as APL-509), ONC392, IBB 10, CG0161, BCD145, ADU1604, AGEN1884 and CS1002. In certain embodiments -D is ipilimumab. In certain embodiments -D is tremelimumab.

Exemplary Fc enhanced for effector function/FcγR binding anti-CTLA4 antibodies are selected from the group consisting of AGEN1181 and anti-CTLA-4 SIFbody.

Exemplary anti-CTLA4 antibodies conditionally active in tumor microenvironment are selected from the group consisting of BMS-986249 and BA3071.

An exemplary anti-CTLA4 small molecules is BPI-002. An exemplary CTLA4 antagonist fusion protein is FPT155.

An exemplary anti CTLA4 anticalin is PRS010.

Exemplary anti-CTLA4 multispecific biologies are selected from the group consisting of TE1254, XmAb22841, XmAb20717, MEDI5752, MGD019, ALPN-202, ATOR-1015 and ATOR-1144.

If the conjugates of the present comprise more than one type of -D, all -D may be connected to the same type of -L¹- or may be connected to different types of -L¹-, i.e. a first type of -D may be connected to a first type of -L¹-, a second type of -D may be connected to a second type of -L¹- and so on. Using different types of -L¹- may in certain embodiments allow different release kinetics for different types of -D, such as for example a faster release for a first type of -D, a medium release for a second type of -D and a slow release for a third type of -D or any other combination. Accordingly, in certain embodiments the conjugates of the present invention comprise one type of -D. In certain embodiments the conjugates of the present invention comprise two types of -D. In certain embodiments the conjugates of the present invention comprise three types of -D. In certain embodiments the conjugates of the present invention comprise four types of -D.

The moiety -L¹- is conjugated to -D via a functional group of -D, which functional group is in certain embodiments selected from the group consisting of carboxylic acid, primary amine, secondary amine, thiol, sulfonic acid, carbonate, carbamate, hydroxyl, aldehyde, ketone, hydrazine, isothiocyanate, phosphoric acid, phosphonic acid, acryloyl, hydroxylamine, sulfate, vinyl sulfone, vinyl ketone, diazoalkane, guanidine, aziridine, amide, imide, imine, urea, amidine, guanidine, sulfonamide, phosphonamide, phorphoramide, hydrazide and selenol. In certain embodiments -L¹- is conjugated to -D via a functional group of -D selected from the group consisting of carboxylic acid, primary amine, secondary amine, thiol, sulfonic acid, carbonate, carbamate, hydroxyl, aldehyde, ketone, hydrazine, isothiocyanate, phosphoric acid, phosphonic acid, acryloyl, hydroxylamine, sulfate, vinyl sulfone, vinyl ketone, diazoalkane, guanidine, amidine and aziridine. In certain embodiments -L¹- is conjugated to -D via a functional group of -D selected from the group consisting of hydroxyl, primary amine, secondary amine, amidine and carboxylic acid.

In certain embodiments -L¹- is conjugated to -D via a hydroxyl group of -D.

In certain embodiments -L¹- is conjugated to -D via a primary amine group of -D.

In certain embodiments -L¹- is conjugated to -D via a secondary amine group of -D.

In certain embodiments -L¹- is conjugated to -D via a carboxylic acid group of -D.

In certain embodiments -L¹- is conjugated to -D via an amidine group of -D.

The moiety -L¹- can be connected to -D through any type of linkage, provided that it is reversible. In certain embodiments -L¹- is connected to -D through a linkage selected from the group consisting of amide, ester, carbamate, acetal, aminal, imine, oxime, hydrazone, disulfide, acylguanidine, acylamidine, carbonate, phosphate, sulfate, urea, hydrazide, thioester, thiophosphate, thiosulfate, sulfonamide, sulfoamidine, sulfaguanidine, phosphoramide, phosphoamidine, phosphoguanidine, phosphonamide, phosphonamidine, phosphonguanidine, phosphonate, borate and imide. In certain embodiments -L¹- is connected to -D through a linkage selected from the group consisting of amide, ester, carbonate, carbamate, acetal, aminal, imine, oxime, hydrazone, disulfide, acylamidine and acylguanidine. In certain embodiments -L¹- is connected to -D through a linkage selected from the group consisting of amide, ester, caronate, acylamide and carbamate. It is understood that some of these linkages may not be reversible per se, but that in the present invention neighboring groups comprised in -L¹- render these linkage reversible.

In certain embodiments -L¹- is connected to -D through an ester linkage.

In certain embodiments -L¹- is connected to -D through a carbonate linkage.

In certain embodiments -L¹- is connected to -D through an acylamidine linkage.

In certain embodiments -L¹- is connected to -D through a carbamate linkage.

In certain embodiments -L¹- is connected to -D through an amide linkage.

If -D is daptomycin, -L¹- is in certain embodiments connected via the primary amine of the ornithine side chain. In certain embodiments such daptomycin is connected to -L¹- via the primary amine of the ornithine side chain via an amide linkage.

It is understood that the conjugates of the present invention are prodrugs.

The moiety -L¹- is a linker moiety from which -D is preferably released in its free form, i.e. in the form of D-H or D-OH. Such moieties are also known as “prodrug linkers” or “reversible prodrug linkers” and are known in the art, such as for example the reversible linker moieties disclosed in WO 2005/099768 A2, WO 2006/136586 A2, WO 2011/089216 A1, WO 2013/024053 A1, WO 2011/012722 A1, WO 2011/089214 A1, WO 2011/089215 A1, WO 2013/024052 A1 and WO 2013/160340 A1, which are incorporated by reference herewith.

In certain embodiments the moiety -L¹- is as disclosed in WO 2009/095479 A2. Accordingly, in certain embodiments the moiety -L¹- is of formula (I):

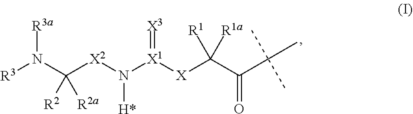

wherein the dashed line indicates the attachment to a nitrogen, hydroxyl or thiol of -D; —X— is selected from the group consisting of —C(R⁴R^{4a})—, —N(R⁴)—, —O—, —C(R⁴R^{4a})—C(R⁵R^{5a})—, —C(R⁵R^{5a})—C(R⁴R^{4a})—, —C(R⁴R^{4a})—N(R⁶)—, —N(R⁶)—C(R⁴R^{4a})—, —C(R⁴R^{4a})—O—, —O—C(R⁴R^{4a})—, and —C(R⁷R^{7a})—,
X¹ is selected from the group consisting of C and S(O);
—X²— is selected from the group consisting of —C(R⁸R^{8a})— and —C(R⁸R^{8a})—C(R⁹R^{9a})—;
═X³ is selected from the group consisting of ═O, ═S, and ═N—CN;
—R¹, —R^{1a}, —R², —R^{2a}, —R⁴, —R^{4a}, —R⁵, —R^{5a}, —R⁶, —R⁸, —R^{8a}, —R⁹ and —R^{9a} are independently selected from the group consisting of —H and C₁₋₆ alkyl;
—R³ and —R^{3a} are independently selected from the group consisting of —H and C₁₋₆ alkyl, provided that in case one of —R³ and —R^{3a} or both are other than —H they are connected to N to which they are attached through an sp³-hybridized carbon atom;
—R⁷ is selected from the group consisting of —N(R¹⁰R^{10a}) and —NR¹⁰—(C═O)—R¹¹;
—R^{7a}, —R¹⁰, —R^{10a} and —R¹¹ are independently selected from the group consisting of —H and C₁₋₆ alkyl;
optionally, one or more of the pairs —R^{1a}/—R^{4a}, —R^{1a}/—R^{5a}, —R^{1a}/—R^{7a}, —R^{4a}/—R^{5a} and —R^{8a}/—R^{9a} form a chemical bond;
optionally, one or more of the pairs —R¹/—R^{1a}, —R²/—R^{2a}, —R⁴/—R^{4a}, —R⁵/—R^{5a}, —R⁸/—R^{8a} and —R⁹/—R^{9a} are joined together with the atom to which they are attached to form a C3-10 cycloalkyl or 3- to 10-membered heterocyclyl;
optionally, one or more of the pairs —R¹/—R⁴, —R¹/—R⁵, —R¹/—R⁶, —R¹/—R^{7a}, —R⁴/—R⁵, —R⁴/—R⁶, —R⁸/—R⁹ and —R²/—R³ are joined together with the atoms to which they are attached to form a ring A;
optionally, R³/R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein -L¹- is substituted with -L²- and wherein -L¹- is optionally further substituted, provided that the hydrogen marked with the asterisk in formula (I) is not replaced by -L²- or a substituent.

The optional further substituents of -L¹- of formula (I) are as described above.

In certain embodiments -L¹- of formula (I) is substituted with one moiety -L²-.

In certain embodiments -L¹- of formula (I) is not further substituted.

It is understood that if —R³/—R^{3a} of formula (I) are joined together with the nitrogen atom to which they are attached to form a 3- to 10-membered heterocycle, only such 3- to 10-membered heterocycles may be formed in which the atoms directly attached to the nitrogen are sp³-hybridized carbon atoms. In other words, such 3- to 10-membered heterocycle formed by —R³/—R^{3a} together with the nitrogen atom to which they are attached has the following structure:

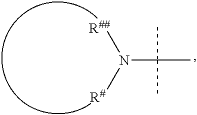

wherein
the dashed line indicates attachment to the rest of -L¹-;
the ring comprises 3 to 10 atoms comprising at least one nitrogen; and
R^{#} and R^{##} represent an sp³-hydridized carbon atom.

It is also understood that the 3- to 10-membered heterocycle may be further substituted.

Exemplary embodiments of suitable 3- to 10-membered heterocycles formed by —R³/—R^{3a} of formula (I) together with the nitrogen atom to which they are attached are the following:

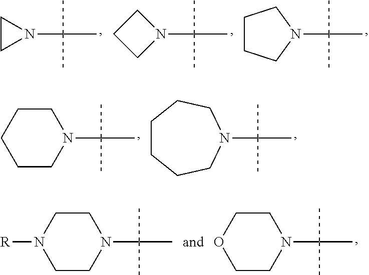

wherein
dashed lines indicate attachment to the rest of the molecule; and
—R is selected from the group consisting of —H and C₁₋₆ alkyl.

-L¹- of formula (I) may optionally be further substituted. In general, any substituent may be used as far as the cleavage principle is not affected, i.e. the hydrogen marked with the asterisk in formula (I) is not replaced and the nitrogen of the moiety

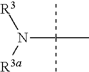

of formula (I) remains part of a primary, secondary or tertiary amine, i.e. —R³ and —R^{3a} are independently of each other —H or are connected to —N<through an sp³-hybridized carbon atom.

In certain embodiments —R¹ or —R^{1a} of formula (I) is substituted with -L²-. In certain embodiments —R² or —R^{2a} of formula (I) is substituted with -L²-. In certain embodiments —R³ or —R^{3a} of formula (I) is substituted with -L²-. In certain embodiments —R⁴ of formula (I) is substituted with -L²-. In certain embodiments —R⁵ or —R^{5a} of formula (I) is substituted with -L²-. In certain embodiments —R⁶ of formula (I) is substituted with -L²-. In certain embodiments —R⁷ or —R^{7a} of formula (I) is substituted with -L²-. In certain embodiments —R⁸ or —R^{8a} of formula (I) is substituted with -L²-. In certain embodiments —R⁹ or —R^{9a} of formula (I) is substituted with -L²-. In certain embodiments —R¹⁰ is substituted with -L²-. In certain embodiments —R¹¹ is substituted with -L²-.

In certain embodiments —X— of formula (I) is selected from the group consisting of —C(R⁴R^{4a})—, —N(R⁴)— and —C(R⁷R^{7a})—.

In certain embodiments —X— of formula (I) is —C(R⁴R^{4a})—.

In certain embodiments —X— of formula (I) is —N(R⁴)—.

In certain embodiments —X— of formula (I) is —C(R⁷R^{7a})—.

In certain embodiments —R⁷ of formula (I) is —NR¹⁰—(C═O)—R¹¹.

In certain embodiments —R^{7a} of formula (I) is selected from —H, methyl and ethyl.

In certain embodiments —R^{7a} of formula (I) is —H.

In certain embodiments —R¹⁰ of formula (I) is selected from —H, methyl and ethyl.

In certain embodiments —R¹⁰ of formula (I) is methyl. In certain embodiments —R¹⁰ is —H.

In certain embodiments —R^{10a} of formula (I) is selected from —H, methyl and ethyl.

In certain embodiments —R^{10a} of formula (I) is methyl. In certain embodiments —R^{10a} is —H.

In certain embodiments —R¹¹ of formula (I) is selected from —H, methyl and ethyl. In certain embodiments —R¹¹ is —H.

In certain embodiments —R¹¹ of formula (I) is substituted with -L²-.

In certain embodiments X¹ of formula (I) is C.

In certain embodiments ═X³ of formula (I) is ═O.

In certain embodiments —X²— of formula (I) is —C(R⁸R^{8a})—.

In certain embodiments —X²— of formula (I) is —C(R⁸R^{8a})—C(R⁹R^{9a})—. In certain embodiments —R⁸ and —R^{8a} of formula (I) are independently selected from the group consisting of —H, methyl and ethyl. In certain embodiments at least one of —R⁸ and —R^{8a} of formula (I) is —H. In certain embodiments both —R⁸ and —R^{8a} of formula (I) are —H.

In certain embodiments —R¹ and —R^{1a} of formula (I) are independently selected from the group consisting of —H, methyl and ethyl. In certain embodiments at least one of —R¹ and —R^{1a} of formula (I) is —H. In certain embodiments both —R¹ and —R^{1a} of formula (I) are —H.

In certain embodiments —R² and —R^{2a} of formula (I) are independently selected from the group consisting of —H, methyl and ethyl. In certain embodiments at least one of —R² and —R^{2a} of formula (I) is —H. In certain embodiments both —R² and —R^{2a} of formula (I) are H.

In certain embodiments —R³ and —R^{3a} of formula (I) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments at least one of —R³ and —R^{3a} of formula (I) is —H. In certain embodiments both —R³ and —R^{3a} of formula (I) are —H. In certain embodiments at least one of —R³ and —R^{3a} of formula (I) is methyl. In certain embodiments both —R³ and —R^{3a} of formula (I) are methyl.

In certain embodiments —R⁴ and —R^{4a} of formula (I) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments at least one of —R⁴ and —R^{4a} of formula (I) is —H. In certain embodiments both —R⁴ and —R^{4a} of formula (I) are —H. In certain embodiments at least one of —R⁴ and —R^{4a} of formula (I) is methyl. In certain embodiments both —R⁴ and —R^{4a} of formula (I) are methyl.

In certain embodiments —R⁵ and —R^{5a} of formula (I) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments at least one of —R⁵ and —R^{5a} of formula (I) is —H. In certain embodiments both —R⁵ and —R^{5a} of formula (I) are —H. In certain embodiments at least one of —R⁵ and —R^{5a} of formula (I) is methyl. In certain embodiments both —R⁵ and —R^{5a} of formula (I) are methyl.

In certain embodiments —R⁶ of formula (I) is selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments —R⁶ of formula (I) is —H. In certain embodiments —R⁶ of formula (I) is methyl.

In certain embodiments —R⁹ and —R^{9a} of formula (I) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments at least one of —R⁹ and —R^{9a} of formula (I) is —H. In certain embodiments both —R⁹ and —R^{9a} of formula (I) are —H. In certain embodiments at least one of —R⁹ and —R^{9a} of formula (I) is methyl. In certain embodiments both —R⁹ and —R^{9a} of formula (I) are methyl.

In certain embodiments -D is connected to -L¹- through a nitrogen by forming an amide bond.

In certain embodiments the moiety -L¹- is of formula (Ia):

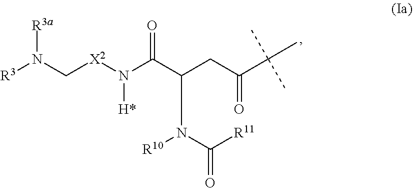

wherein the dashed line indicates the attachment to a nitrogen of -D by forming an amide bond;
—R³, —R^{3a}, —R¹⁰, —R¹¹ and —X²— are used as defined in formula (I); and wherein -L¹- is substituted with -L²- and wherein -L¹- is optionally further substituted, provided that the hydrogen marked with the asterisk in formula (Ia) is not replaced by -L²- or a substituent.

The optional further substituents of -L¹- of formula (Ia) are as described above.

In certain embodiments -L¹- of formula (Ia) is substituted with one moiety -L²-.

In certain embodiments the moiety -L¹- of formula (Ia) is not further substituted.

In certain embodiments —X²— of formula (Ia) is —C(R⁸R^{8a})—.

In certain embodiments —R⁸ and —R^{8a} of formula (Ia) are independently selected from the group consisting of —H, methyl and ethyl. In certain embodiments at least one of —R⁸ and —R^{8a} of formula (Ia) is —H. In certain embodiments both —R⁸ and —R^{8a} of formula (Ia) are —H.

In certain embodiments —R³ and —R^{3a} of formula (Ia) are independently selected from the group consisting of methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments at least one of —R³ and —R^{3a} of formula (Ia) is methyl. In certain embodiments both —R³ and —R^{3a} of formula (Ia) are methyl.

In certain embodiments —R¹⁰ of formula (Ia) is selected from —H, methyl and ethyl. In certain embodiments —R¹⁰ of formula (Ia) is methyl.

In certain embodiments —R¹¹ of formula (Ia) is selected from —H, methyl and ethyl. In certain embodiments —R¹¹ of formula (Ia) is —H.

In certain embodiments —R¹¹ of formula (Ia) is substituted with -L²-.

In certain embodiments the moiety -L¹- is of formula (Ib):

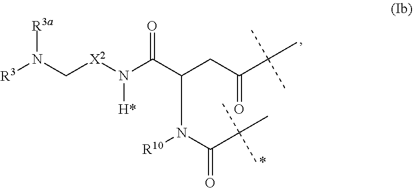

wherein
wherein the dashed line indicates the attachment to a nitrogen of -D by forming an amide bond;
the dashed line marked with the asterisk indicates attachment to -L²-;
—R³, —R^{3a}, —R¹⁰ and —X²— are used as defined in formula (I); and
wherein -L¹- is optionally further substituted, provided that the hydrogen marked with the asterisk in formula (Ib) is not replaced by a substituent.

The optional further substituents of -L¹- of formula (Ib) are as described above.

In certain embodiments the moiety -L¹- of formula (Ib) is not further substituted.

In certain embodiments —X²— of formula (Ib) is —C(R⁸R^{8a})—.

In certain embodiments —R⁸ and —R^{8a} of formula (Ib) are independently selected from the group consisting of —H, methyl and ethyl. In certain embodiments at least one of —R⁸ and —R^{8a} of formula (Ib) is —H. In certain embodiments both —R⁸ and —R^{8a} of formula (Ib) are —H.

In certain embodiments —R³ and —R^{3a} of formula (Ib) are independently selected from the group consisting of —H, methyl, ethyl, n-propyl, isopropyl, n-butyl, isobutyl, sec-butyl, tert-butyl, n-pentyl, 2-methylbutyl, 2,2-dimethylpropyl, n-hexyl, 2-methylpentyl, 3-methylpentyl, 2,2-dimethylbutyl, 2,3-dimethylbutyl and 3,3-dimethylpropyl. In certain embodiments at least one of —R³ and —R^{3a} of formula (Ib) is methyl. In certain embodiments both —R³ and —R^{3a} of formula (Ib) are methyl.

In certain embodiments —R¹⁰ of formula (Ib) is selected from —H, methyl and ethyl. In certain embodiments —R¹⁰ of formula (Ib) is methyl.

In certain embodiments the moiety -L¹- is of formula (Ic):

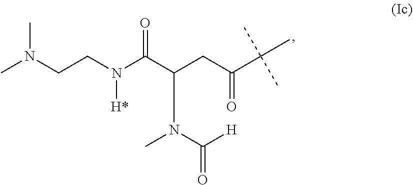

wherein the dashed line indicates the attachment to a nitrogen of -D by forming an amide bond; and
wherein -L¹- is substituted with -L²- and wherein -L¹- is optionally further substituted, provided that the hydrogen marked with the asterisk in formula (Ic) is not replaced by -L²- or a substituent.

The optional further substituents of -L¹- of formula (Ic) are as described above.

In certain embodiments -L¹- of formula (Ic) is substituted with one moiety -L²-.

In certain embodiments the moiety -L¹- of formula (Ic) is not further substituted.

In certain embodiments the moiety -L¹- is of formula (Id):

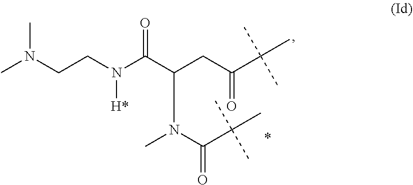

wherein
wherein the dashed line indicates the attachment to a nitrogen of -D by forming an amide bond;
the dashed line marked with the asterisk indicates attachment to -L²-; and
wherein -L¹- is optionally further substituted, provided that the hydrogen marked with the asterisk in formula (Id) is not replaced by a substituent.

In certain embodiments the moiety -L¹- of formula (Id) is not further substituted.

In certain embodiments -L¹- is disclosed in WO 2016/020373 A1. Accordingly, in certain embodiments the moiety -L¹- is of formula (II):

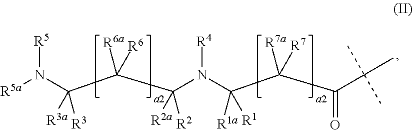

wherein
the dashed line indicates attachment to a primary or secondary amine or hydroxyl of -D by forming an amide or ester linkage, respectively;
—R¹, —R^{1a}, —R², —R^{2a}, —R³ and —R^{3a} are independently of each other selected from the group consisting of —H, —C(R⁸R^{8a}R^{8b}), —C(═O)R⁸, —C≡N, —C(═NR⁸)R^{8a}, —CR⁸(═CR^{8a}R^{8b}), —C≡CR⁸ and -T;
—R⁴, —R⁵ and —R^{5a} are independently of each other selected from the group consisting of —H, —C(R⁹R^{9a}R^{9b}) and -T;
a1 and a2 are independently of each other 0 or 1;
each —R⁶, —R^{6a}, —R⁷, —R^{7a}, —R⁸, —R^{8a}, —R^{8b}, —R⁹, —R^{9a}, —R^{9b} are independently of each other selected from the group consisting of —H, halogen, —CN, —COOR¹⁰, —OR¹⁰, —C(O)R¹⁰, —C(O)N(R¹⁰R^{10a}), —S(O)₂N(R¹⁰R^{10a}), —S(O) N(R¹⁰R^{10a}), —S(O)₂R¹⁰, —S(O)R¹⁰, —N(R¹⁰)S(O)₂N(R^{10a}R^{10b}), —SR¹⁰, —N(R¹⁰R^{10a}), —NO₂, —OC(O)R¹⁰, —N(R¹⁰)C(O)R^{10a}, —N(R¹⁰)S(O)₂R^{10a}, —N(R¹⁰)S(O)R^{10a}, —N(R¹⁰)C(O)OR^{10a}, —N(R¹⁰)C(O)N(R^{10a}R^{10b}), —OC(O)N(R¹⁰R^{10a}), -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each —R¹⁰, —R^{10a}, —R^{10b} is independently selected from the group consisting of —H, -T, C₁₋₂₀ alkyl, C₂₋₂₀ alkenyl, and C₂₋₂₀ alkynyl; wherein -T, C₁₋₂₀ alkyl, C₂₋₂₀ alkenyl, and C₂₋₂₀ alkynyl are optionally substituted with one or more —R¹¹, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R¹²)—, —S(O)₂N(R¹²)—, —S(O)N(R¹²)—, —S(O)₂—, —S(O)—, —N(R¹²)S(O)₂N(R^{12a})—, —S—, —N(R¹²)—, —OC(OR¹²)(R^{12a})—, —N(R¹²)C(O)N(R^{12a})—, and —OC(O)N(R¹²)—;
each T is independently of each other selected from the group consisting of phenyl, naphthyl, indenyl, indanyl, tetralinyl, C₃₋₁₀ cycloalkyl, 3- to 10-membered heterocyclyl, and 8- to 11-membered heterobicyclyl; wherein each T is independently optionally substituted with one or more —R¹¹, which are the same or different;
each —R¹¹ is independently of each other selected from halogen, —CN, oxo (═O), —COOR¹³, —OR¹³, —C(O)R¹³, —C(O)N(R¹³R^{13a}), —S(O)₂N(R¹³R^{13a}), —S(O)N(R¹³R^{13a}), —S(O)₂R¹³, —S(O)R¹³, —N(R¹³)S(O)₂N(R^{13a}R^{13b}), —SR¹³, —N(R¹³R^{13a}), —NO₂, —OC(O)R¹³, —N(R¹³)C(O)R^{13a}, —N(R¹³)S(O)₂R^{13a}, —N(R¹³)S(O)R^{13a}, —N(R¹³)C(O)OR^{13a}, —N(R¹³)C(O)N(R^{13a}R^{13b}), —OC(O)N(R¹³R^{13a}), and C₁₋₆ alkyl; wherein C₁₋₆ alkyl is optionally substituted with one or more halogen, which are the same or different;
each —R¹², —R^{12a}, —R¹³, —R^{13a}, —R^{13b} is independently selected from the group consisting of —H, and C₁₋₆ alkyl; wherein C₁₋₆ alkyl is optionally substituted with one or more halogen, which are the same or different;
optionally, one or more of the pairs —R¹/—R^{1a}, —R²/—R^{2a}, —R³/—R^{3a}, —R⁶/—R^{6a}, —R⁷/—R^{7a} are joined together with the atom to which they are attached to form a C₃₋₁₀ cycloalkyl or a 3- to 10-membered heterocyclyl;
optionally, one or more of the pairs —R¹/—R², —R¹/—R³, —R¹/—R⁴, —R¹/—R⁵, —R¹/—R⁶, —R¹—R⁷, —R²/—R³, —R²/—R⁴, —R²/—R⁵, —R²/—R⁶, —R²/—R⁷, —R³/—R⁴, —R³/—R⁵, —R³/—R⁶, —R³/—R⁷, —R⁴/—R⁵, —R⁴/—R⁶, —R⁴/—R⁷, —R⁵/—R⁶, —R⁵/—R⁷, —R⁶/—R⁷ are joint together with the atoms to which they are attached to form a ring A;
A is selected from the group consisting of phenyl; naphthyl; indenyl; indanyl; tetralinyl; C3-10 cycloalkyl; 3- to 10-membered heterocyclyl; and 8- to 11-membered heterobicyclyl; and
wherein -L¹- is substituted with -L²- and wherein -L¹- is optionally further substituted. The optional further substituents of -L¹- of formula (II) are as described above.

In certain embodiments -L¹- of formula (II) is substituted with one moiety -L²-.

In certain embodiments -L¹- of formula (II) is not further substituted.

Additional embodiments for -L¹- are disclosed in EP1536334B1, WO2009/009712A1, WO2008/034122A1, WO2009/143412A2, WO2011/082368A2, and U.S. Pat. No. 8,618,124B2, which are herewith incorporated by reference in their entirety.

Further embodiments for -L¹- are disclosed in U.S. Pat. No. 8,946,405B2 and U.S. Pat. No. 8,754,190B2, which are herewith incorporated by reference in their entirety. Accordingly, in certain embodiments -L¹- is of formula (III):

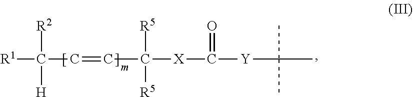

wherein
the dashed line indicates attachment to -D through a functional group of -D selected from the group consisting of —OH, —SH and —NH₂;
m is 0 or 1;
at least one or both of —R¹ and —R² is/are independently of each other selected from the group consisting of —CN, —NO₂, optionally substituted aryl, optionally substituted heteroaryl, optionally substituted alkenyl, optionally substituted alkynyl, —C(O)R³, —S(O)R³, —S(O)₂R³, and —SR⁴,
one and only one of —R¹ and —R² is selected from the group consisting of —H, optionally substituted alkyl, optionally substituted arylalkyl, and optionally substituted heteroarylalkyl;
—R³ is selected from the group consisting of —H, optionally substituted alkyl, optionally substituted aryl, optionally substituted arylalkyl, optionally substituted heteroaryl, optionally substituted heteroarylalkyl, —OR⁹ and —N(R⁹)₂;
—R⁴ is selected from the group consisting of optionally substituted alkyl, optionally substituted aryl, optionally substituted arylalkyl, optionally substituted heteroaryl, and optionally substituted heteroarylalkyl;
each —R⁵ is independently selected from the group consisting of —H, optionally substituted alkyl, optionally substituted alkenylalkyl, optionally substituted alkynylalkyl, optionally substituted aryl, optionally substituted arylalkyl, optionally substituted heteroaryl and optionally substituted heteroarylalkyl;
—R⁹ is selected from the group consisting of —H and optionally substituted alkyl;
—Y— is absent and —X— is —O— or —S—; or
—Y— is —N(Q)CH₂— and —X— is —O—;
Q is selected from the group consisting of optionally substituted alkyl, optionally substituted aryl, optionally substituted arylalkyl, optionally substituted heteroaryl and optionally substituted heteroarylalkyl;
optionally, —R¹ and —R² may be joined to form a 3 to 8-membered ring; and optionally, both —R⁹ together with the nitrogen to which they are attached form a heterocyclic ring; and
wherein -L¹- is substituted with -L²- and wherein -L¹- is optionally further substituted.

Only in the context of formula (III) the terms used have the following meaning: The term “alkyl” as used herein includes linear, branched or cyclic saturated hydrocarbon groups of 1 to 8 carbon atoms, or in some embodiments 1 to 6 or 1 to 4 carbon atoms.

The term “alkoxy” includes alkyl groups bonded to oxygen, including methoxy, ethoxy, isopropoxy, cyclopropoxy, cyclobutoxy, and similar.

The term “alkenyl” includes non-aromatic unsaturated hydrocarbons with carbon-carbon double bonds.

The term “alkynyl” includes non-aromatic unsaturated hydrocarbons with carbon-carbon triple bonds.

The term “aryl” includes aromatic hydrocarbon groups of 6 to 18 carbons, preferably 6 to 10 carbons, including groups such as phenyl, naphthyl, and anthracenyl. The term “heteroaryl” includes aromatic rings comprising 3 to 15 carbons containing at least one N, O or S atom, preferably 3 to 7 carbons containing at least one N, O or S atom, including groups such as pyrrolyl, pyridyl, pyrimidinyl, imidazolyl, oxazolyl, isoxazolyl, thiazolyl, isothiazolyl, quinolyl, indolyl, indenyl, and similar.

In some instance, alkenyl, alkynyl, aryl or heteroaryl moieties may be coupled to the remainder of the molecule through an alkylene linkage. Under those circumstances, the substituent will be referred to as alkenylalkyl, alkynylalkyl, arylalkyl or heteroarylalkyl, indicating that an alkylene moiety is between the alkenyl, alkynyl, aryl or heteroaryl moiety and the molecule to which the alkenyl, alkynyl, aryl or heteroaryl is coupled.

The term “halogen” includes bromo, fluoro, chloro and iodo.

The term “heterocyclic ring” refers to a 4 to 8 membered aromatic or non-aromatic ring comprising 3 to 7 carbon atoms and at least one N, O, or S atom. Examples are piperidinyl, piperazinyl, tetrahydropyranyl, pyrrolidine, and tetrahydrofuranyl, as well as the exemplary groups provided for the term “heteroaryl” above.

When a ring system is optionally substituted, suitable substituents are selected from the group consisting of alkyl, alkenyl, alkynyl, or an additional ring, each optionally further substituted. Optional substituents on any group, including the above, include halo, nitro, cyano, —OR, —SR, —NR₂, —OCOR, —NRCOR, —COOR, —CONR₂, —SOR, —SO₂R, —SONR₂, —SO₂N R₂, wherein each R is independently alkyl, alkenyl, alkynyl, aryl or heteroaryl, or two R groups taken together with the atoms to which they are attached form a ring.

In certain embodiments -L¹- of formula (III) is substituted with one moiety -L²-.

Another embodiment for -L¹- is disclosed in WO2013/036857A1, which is herewith incorporated by reference in its entirety. Accordingly, in certain embodiments -L¹- is of formula (IV):

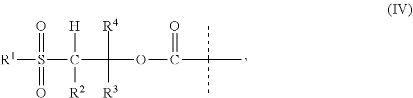

wherein
the dashed line indicates attachment to -D through an amine functional group of -D;
—R¹ is selected from the group consisting of optionally substituted C₁-C₆ linear, branched, or cyclic alkyl; optionally substituted aryl; optionally substituted heteroaryl; alkoxy; and —NR⁵₂;
—R² is selected from the group consisting of —H; optionally substituted C₁-C₆ alkyl; optionally substituted aryl; and optionally substituted heteroaryl;
—R³ is selected from the group consisting of —H; optionally substituted C₁-C₆ alkyl; optionally substituted aryl; and optionally substituted heteroaryl;
—R⁴ is selected from the group consisting of —H; optionally substituted C₁-C₆ alkyl; optionally substituted aryl; and optionally substituted heteroaryl;
each —R⁵ is independently of each other selected from the group consisting of —H; optionally substituted C₁-C₆ alkyl; optionally substituted aryl; and optionally substituted heteroaryl; or when taken together two —R⁵ can be cycloalkyl or cycloheteroalkyl; and
wherein -L¹- is substituted with -L²- and wherein -L¹- is optionally further substituted.

Only in the context of formula (IV) the terms used have the following meaning:

“Alkyl”, “alkenyl”, and “alkynyl” include linear, branched or cyclic hydrocarbon groups of 1-8 carbons or 1-6 carbons or 1-4 carbons wherein alkyl is a saturated hydrocarbon, alkenyl includes one or more carbon-carbon double bonds and alkynyl includes one or more carbon-carbon triple bonds. Unless otherwise specified these contain 1-6 C.

“Aryl” includes aromatic hydrocarbon groups of 6-18 carbons, preferably 6-10 carbons, including groups such as phenyl, naphthyl, and anthracene “Heteroaryl” includes aromatic rings comprising 3-15 carbons containing at least one N, O or S atom, preferably 3-7 carbons containing at least one N, O or S atom, including groups such as pyrrolyl, pyridyl, pyrimidinyl, imidazolyl, oxazolyl, isoxazolyl, thiszolyl, isothiazolyl, quinolyl, indolyl, indenyl, and similar.

The term “substituted” means an alkyl, alkenyl, alkynyl, aryl, or heteroaryl group comprising one or more substituent groups in place of one or more hydrogen atoms. Substituents may generally be selected from halogen including F, Cl, Br, and I; lower alkyl including linear, branched, and cyclic; lower haloalkyl including fluoroalkyl, chloroalkyl, bromoalkyl, and iodoalkyl; OH; lower alkoxy including linear, branched, and cyclic; SH; lower alkylthio including linear, branched and cyclic; amino, alkylamino, dialkylamino, silyl including alkylsilyl, alkoxysilyl, and arylsilyl; nitro; cyano; carbonyl; carboxylic acid, carboxylic ester, carboxylic amide, aminocarbonyl; aminoacyl; carbamate; urea; thiocarbamate; thiourea; ketne; sulfone; sulfonamide; aryl including phenyl, naphthyl, and anthracenyl; heteroaryl including 5-member heteroaryls including as pyrrole, imidazole, furan, thiophene, oxazole, thiazole, isoxazole, isothiazole, thiadiazole, triazole, oxadiazole, and tetrazole, 6-member heteroaryls including pyridine, pyrimidine, pyrazine, and fused heteroaryls including benzofuran, benzothiophene, benzoxazole, benzimidazole, indole, benzothiazole, benzisoxazole, and benzisothiazole.

In certain embodiments -L¹- of formula (IV) is substituted with one moiety -L²-.

A further embodiment for -L¹- is disclosed in U.S. Pat. No. 7,585,837B2, which is herewith incorporated by reference in its entirety. Accordingly, in certain embodiments -L¹- is of formula (V):

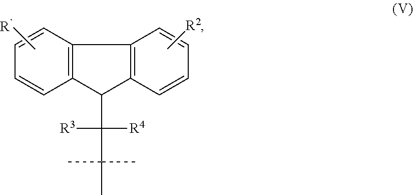

wherein
the dashed line indicates attachment to -D through an amine functional group of -D; R¹ and R² are independently selected from the group consisting of hydrogen, alkyl, alkoxy, alkoxyalkyl, aryl, alkaryl, aralkyl, halogen, nitro, —SO₃H, —SO₂NHR⁵, amino, ammonium, carboxyl, PO₃H₂, and OPO₃H₂;
R³, R⁴, and R⁵ are independently selected from the group consisting of hydrogen, alkyl, and aryl; and
wherein -L¹- is substituted with -L²- and wherein -L¹- is optionally further substituted.

Suitable substituents for formulas (V) are alkyl (such as C₁₋₆ alkyl), alkenyl (such as C₂₋₆ alkenyl), alkynyl (such as C₂₋₆ alkynyl), aryl (such as phenyl), heteroalkyl, heteroalkenyl, heteroalkynyl, heteroaryl (such as aromatic 4 to 7 membered heterocycle) or halogen moieties.

Only in the context of formula (V) the terms used have the following meaning:

The terms “alkyl”, “alkoxy”, “alkoxyalkyl”, “aryl”, “alkaryl” and “aralkyl” mean alkyl radicals of 1-8, preferably 1-4 carbon atoms, e.g. methyl, ethyl, propyl, isopropyl and butyl, and aryl radicals of 6-10 carbon atoms, e.g. phenyl and naphthyl. The term “halogen” includes bromo, fluoro, chloro and iodo.

In certain embodiments -L¹- of formula (V) is substituted with one moiety -L²-.

In certain embodiments -L¹- of formula (V) is not further substituted.

A further embodiment for -L¹- is disclosed in WO2002/089789A1, which is herewith incorporated by reference in its entirety. Accordingly, in certain embodiments -L¹- is of formula (VI):

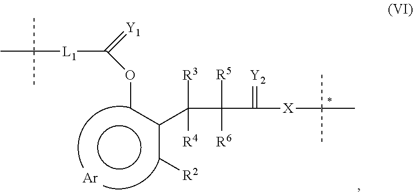

wherein
the dashed line indicates attachment to -D through an amine functional group of -D;
L¹ is a bifunctional linking group,
Y₁ and Y₂ are independently O, S or NR⁷;
R², R³, R⁴, R⁵, R⁶ and R⁷ are independently selected from the group consisting of hydrogen, C₁₋₆ alkyls, C₃₋₁₂ branched alkyls, C₃₋₈ cycloalkyls, C₁₋₆ substituted alkyls, C₃₋₈ substituted cycloalkyls, aryls, substituted aryls, aralkyls, C₁₋₆ heteroalkyls, substituted C₁₋₆ heteroalkyls, C₁₋₆ alkoxy, phenoxy, and C₁₋₆ heteroalkoxy;
Ar is a moiety which when included in formula (VI) forms a multisubstituted aromatic hydrocarbon or a multi-substituted heterocyclic group;
X is a chemical bond or a moiety that is actively transported into a target cell, a hydrophobic moiety, or a combination thereof,
y is 0 or 1; and
wherein -L¹- is substituted with -L²- and wherein -L¹- is optionally further substituted.

Only in the context of formula (VI) the terms used have the following meaning:

The term “alkyl” shall be understood to include, e.g. straight, branched, substituted C₁₋₁₂ alkyls, including alkoxy, C₃₋₈ cycloalkyls or substituted cycloalkyls, etc.

The term “substituted” shall be understood to include adding or replacing one or more atoms contained within a functional group or compounds with one or more different atoms.

Substituted alkyls include carboxyalkyls, aminoalkyls, dialkylaminos, hydroxyalkyls and mercaptoalkyls; substituted cycloalkyls include moieties such as 4-chlorocyclohexyl; aryls include moieties such as napthyl; substituted aryls include moieties such as 3-bromo-phenyl; aralkyls include moieties such as toluyl; heteroalkyls include moieties such as ethylthiophene; substituted heteroalkyls include moieties such as 3-methoxythiophone; alkoxy includes moieities such as methoxy; and phenoxy includes moieties such as 3-nitrophenoxy. Halo- shall be understood to include fluoro, chloro, iodo and bromo.

In certain embodiments -L¹- of formula (VI) is substituted with one moiety -L²-.

In certain embodiments -L¹- of formula (VI) is not further substituted.

In certain embodiments -L¹- comprises a substructure of formula (VII)

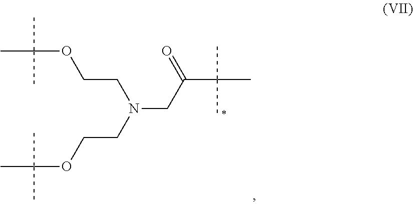

wherein
the dashed line marked with the asterisk indicates attachment to a nitrogen of -D by forming an amide bond;
the unmarked dashed lines indicate attachment to the remainder of -L¹-; and wherein -L¹- is substituted with -L²- and wherein -L¹- is optionally further substituted.

The optional further substituents of -L¹- of formula (VII) are as described above.

In certain embodiments -L¹- of formula (VII) is substituted with one moiety -L²-.

In certain embodiments -L¹- of formula (VII) is not further substituted.

In certain embodiments -L¹- comprises a substructure of formula (VIII)

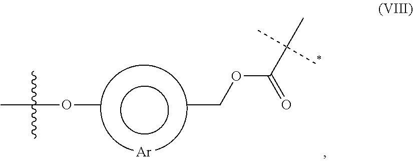

wherein
the dashed line marked with the asterisk indicates attachment to a nitrogen of -D by forming a carbamate bond;
the unmarked dashed lines indicate attachment to the remainder of -L¹-; and wherein -L¹- is substituted with -L²- and wherein -L¹- is optionally further substituted.

The optional further substituents of -L¹- of formula (VIII) are as described above.

In certain embodiments -L¹- of formula (VIII) is substituted with one moiety -L²-.

In certain embodiments -L¹- of formula (VIII) is not further substituted.

In certain embodiments -L¹- is of formula (IX)

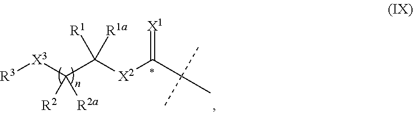

wherein
the dashed line indicates the attachment to a π-electron-pair-donating heteroaromatic N of -D;
n is an integer selected from the group consisting of 0, 1, 2, 3 and 4;
═X¹ is selected from the group consisting of ═O, ═S and ═N(R⁴);
—X²— is selected from the group consisting of —O—, —S—, —N(R⁵)— and —C(R⁶)(R^{6a})—;

—X³— is selected from the group consisting of

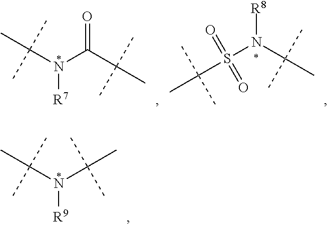

—C(R¹⁰)(R^{10a})—, —C(R¹¹)(R^{11a})—C(R¹²)(R^{12a})—, —O— and —C(O)—;
—R¹, —R^{1a}, —R⁶, —R^{6a}, —R¹⁰, —R^{10a}, —Rⁿ, —R^{11a}, —R¹², —R^{12a} and each of —R² and —R^{2a} are independently selected from the group consisting of —H, —C(O)OH, halogen, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
R³, —R⁴, —R⁵, —R⁷, —R⁸ and —R⁹ are independently selected from the group consisting of —H, -T, —CN, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R¹³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(Q)N(R¹⁴)—, —S(O)₂N(R¹⁴)—, —S(O)N(R¹⁴)—, —S(O)₂—, —S(O)—, —N(R¹⁴)S(O)₂N(R^{14a})—, —S—, —N(R¹⁴)—, —OC(OR¹⁴)(R^{14a})—, —N(R¹⁴)C(O)N(R^{14a})— and —OC(O)N(R¹⁴)—;
   each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl; wherein each T is independently optionally substituted with one or more —R¹³, which are the same or different;
   wherein —R¹³ is selected from the group consisting of —H, —NO₂, —OCH₃, —CN, —N(R¹⁴)(R^{14a}), —OH, —C(O)OH and C₁₋₆ alkyl; wherein C₁₋₆ alkyl is optionally substituted with one or more halogen, which are the same or different;
   wherein —R¹⁴ and —R^{14a} are independently selected from the group consisting of —H and C₁₋₆ alkyl; wherein C₁₋₆ alkyl is optionally substituted with one or more halogen, which are the same or different;
optionally, one or more of the pairs —R¹/—R^{1a}, —R²/—R^{2a}, two adjacent R², —R⁶/—R^{6a}, —R¹⁰/—R^{10a}, —R¹¹/—R^{11a} and —R¹²/—R^{12a} are joined together with the atom to which they are attached to form a C₃₋₁₀ cycloalkyl, 3- to 10-membered heterocyclyl or an 8- to 11-membered heterobicyclyl;
optionally, one or more of the pairs —R¹/—R², —R¹/—R⁵, —R¹/—R⁶, —R¹/—R⁹, —R¹/—R¹⁰, —R³/—R^{6a}, —R⁴/—R⁵, —R^{4a}/—R⁵, —R⁴/—R⁶, —R⁵/—R¹⁰, —R⁶/—R¹⁰ and —R^{4a}/—R⁶ are joined together with the atoms to which they are attached to form a ring -A-;
   wherein -A- is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl;
optionally, —R¹ and an adjacent —R² form a carbon-carbon double bond provided that n is selected from the group consisting of 1, 2, 3 and 4;
optionally, two adjacent —R² form a carbon-carbon double bond provided that n is selected from the group consisting of 2, 3 and 4;
provided that if —X²— is —N(R⁵)—, —X³— is selected from the group consisting of

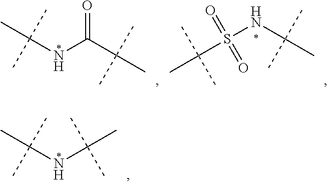

and the distance between the nitrogen atom marked with an asterisk and the carbon atom marked with an asterisk in formula (IX) is 5, 6 or 7 atoms and if present the carbon-carbon double bond formed between —R¹ and —R² or two adjacent —R² is in a cis configuration; and
wherein -L¹- is substituted with -L²- and wherein -L¹- is optionally further substituted.

It is understood that two adjacent —R² in formula (IX) can only exist if n is at least 2.

It is understood that the expression “distance between the nitrogen atom marked with an asterisk and the carbon atom marked with an asterisk” refers to the total number of atoms in the shortest distance between the nitrogen and carbon atoms marked with the asterisk and also includes the nitrogen and carbon atoms marked with the asterisk. For example, in the structure below, n is 1 and the distance between the nitrogen marked with an asterisk and the carbon marked with an asterisk is 5:

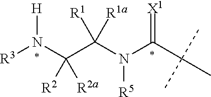

and in the structure below, n is 2, —R¹ and —R^{1a} form a cyclohexal and the distance between the nitrogen marked with an asterisk and the carbon marked with an asterisk is 6:

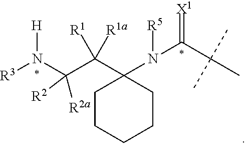

The optional further substituents of -L¹- of formula (IX) are as described elsewhere herein.

In certain embodiments -L¹- of formula (IX) is not further substituted.

In certain embodiments ═X¹ of formula (IX) is ═O. In certain embodiments ═X¹ of formula (IX) is ═S. In certain embodiments ═X¹ of formula (IX) is ═N(R⁴).

In certain embodiments —X²— of formula (IX) is —O—. In certain embodiments —X²— of formula (IX) is —S—. In certain embodiments —X²— of formula (IX) is —N(R⁵)—. In certain embodiments —X²— of formula (IX) is —C(R⁶)(R^{6a})—.

In certain embodiments —X³— of formula (IX) is

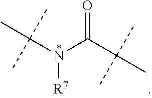

In certain embodiments —X³— of formula (IX) is

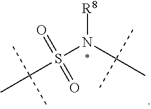

In certain embodiments —X³— of formula (IX) is

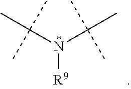

In certain embodiments —X³— of formula (IX) is —C(R¹⁰)(R^{10a})—. In certain embodiments —X³- of formula (IX) is —C(R¹¹)(R^{11a})—C(R¹²)(R^{12a})—. In certain embodiments —X³— of formula (IX) is —O—. In certain embodiments —X³— of formula (IX) is —C(O)—.

In certain embodiments —X²— of formula (IX) is —N(R⁵)—, —X³— is

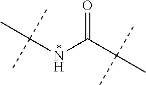

and the distance between the nitrogen atom marked with an asterisk and the carbon atom marked with an asterisk in formula (IX) is 5 atoms.

In certain embodiments —X²— of formula (IX) is —N(R⁵)—, —X³— is

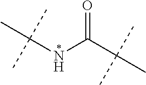

and the distance between the nitrogen atom marked with an asterisk and the carbon atom marked with an asterisk in formula (IX) is 6 atoms.

In certain embodiments —X²— of formula (IX) is —N(R⁵)—, —X³— is

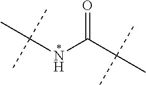

and the distance between the nitrogen atom marked with an asterisk and the carbon atom marked with an asterisk in formula (IX) is 7 atoms.

In certain embodiments —X²— of formula (IX) is —N(R⁵)—, —X³— is

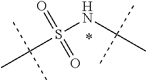

and the distance between the nitrogen atom marked with an asterisk and the carbon atom marked with an asterisk in formula (IX) is 5 atoms.

In certain embodiments —X²— of formula (IX) is —N(R⁵)—, —X³— is

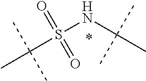

and the distance between the nitrogen atom marked with an asterisk and the carbon atom marked with an asterisk in formula (IX) is 6 atoms.

In certain embodiments —X²— of formula (IX) is —N(R⁵)—, —X³— is

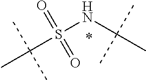

and the distance between the nitrogen atom marked with an asterisk and the carbon atom marked with an asterisk in formula (IX) is 7 atoms.

In certain embodiments —X²— of formula (IX) is —N(R⁵)—, —X³— is

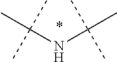

and the distance between the nitrogen atom marked with an asterisk and the carbon atom marked with an asterisk in formula (IX) is 5 atoms.

In certain embodiments —X²— of formula (IX) is —N(R⁵)—, —X³— is

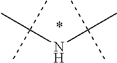

and the distance between the nitrogen atom marked with an asterisk and the carbon atom marked with an asterisk in formula (IX) is 6 atoms.

In certain embodiments —X²— of formula (IX) is —N(R⁵)—, —X³— is

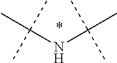

and the distance between the nitrogen atom marked with an asterisk and the carbon atom marked with an asterisk in formula (IX) is 7 atoms.

In certain embodiments ═X¹ of formula (IX) is ═O, —X²— of formula (IX) is —C(R⁶)(R^{6a})—, —X³- of formula (IX) is

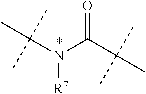

and —R³ of formula (IX) does not comprise an amine.

In certain embodiments —R¹, —R^{1a}, —R⁶, —R^{6a}, —R¹⁰, —R^{10a}, —R¹¹, —R^{11a}, —R¹², —R^{12a} and each of —R² and —R^{2a} of formula (IX) are independently selected from the group consisting of —H, —C(O)OH, halogen, —CN, —OH, C₁₋₆ alkyl, C₂₋₆ alkenyl and C₂₋₆ alkynyl.

In certain embodiments —R¹ of formula (IX) is selected from the group consisting of —H, —C(O)OH, halogen, —CN, —OH, C₁₋₆ alkyl, C₂₋₆ alkenyl and C₂₋₆ alkynyl. In certain embodiments —R¹ of formula (IX) is selected from the group consisting of —H, —C(O)OH, —CN, —OH, C₁₋₆ alkyl, C₂₋₆ alkenyl and C₂₋₆ alkynyl. In certain embodiments —R¹ of formula (IX) is selected from the group consisting of —H, —C(O)OH, halogen, —OH, C₁₋₆ alkyl, C₂₋₆ alkenyl and C₂₋₆ alkynyl. In certain embodiments —R¹ of formula (IX) is selected from the group consisting of —H, —C(O)OH, —OH and C₁₋₆ alkyl. In certain embodiments —R¹ of formula (IX) is —H. In certain embodiments —R¹ of formula (IX) is —C(O)OH. In certain embodiments —R¹ of formula (IX) is halogen. In certain embodiments —R¹ of formula (IX) is —F. In certain embodiments —R¹ of formula (IX) is —CN. In certain embodiments —R¹ of formula (IX) is —OH. In certain embodiments —R¹ of formula (IX) is C₁₋₆ alkyl. In certain embodiments —R¹ of formula (IX) is C₂₋₆ alkenyl. In certain embodiments —R¹ of formula (IX) is C₂₋₆ alkynyl. In certain embodiments —R¹ of formula (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl. In certain embodiments —R^{1a} of formula (IX) is selected from the group consisting of —H, —C(O)OH, halogen, —CN, —OH, C₁₋₆ alkyl, C₂₋₆ alkenyl and C₂₋₆ alkynyl. In certain embodiments —R^{1a} of formula (IX) is selected from the group consisting of —H, —C(O)OH, —CN, —OH, C₁₋₆ alkyl, C₂₋₆ alkenyl and C₂₋₆ alkynyl. In certain embodiments —R^{1a} of formula (IX) is selected from the group consisting of —H, —C(O)OH, halogen, —OH, C₁₋₆ alkyl, C₂₋₆ alkenyl and C₂₋₆ alkynyl. In certain embodiments —R^{1a} of formula (IX) is selected from the group consisting of —H, —C(O)OH, —OH and C₁₋₆ alkyl. In certain embodiments —R^{1a} of formula (IX) is —H. In certain embodiments —R^{1a} of formula (IX) is —C(O)OH. In certain embodiments —R^{1a} of formula (IX) is halogen. In certain embodiments —R^{1a} of formula (IX) is —F. In certain embodiments —R^{1a} of formula (IX) is —CN. In certain embodiments —R^{1a} of formula (IX) is —OH. In certain embodiments —R^{1a} of formula (IX) is C₁₋₆ alkyl. In certain embodiments —R^{1a} of formula (IX) is C₂₋₆ alkenyl. In certain embodiments —R^{1a} of formula (IX) is C₂₋₆ alkynyl. In certain embodiments —R^{1a} of formula (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R⁶ of formula (IX) is selected from the group consisting of —H, —C(O)OH, halogen, —CN, —OH, C₁₋₆ alkyl, C₂₋₆ alkenyl and C₂₋₆ alkynyl. In certain embodiments —R⁶ of formula (IX) is selected from the group consisting of —H, —C(O)OH, —CN, —OH, C₁₋₆ alkyl, C₂₋₆ alkenyl and C₂₋₆ alkynyl. In certain embodiments —R⁶ of formula (IX) is selected from the group consisting of —H, —C(O)OH, halogen, —OH, C₁₋₆ alkyl, C₂₋₆ alkenyl and C₂₋₆ alkynyl. In certain embodiments —R⁶ of formula (IX) is selected from the group consisting of —H, —C(O)OH, —OH and C₁₋₆ alkyl. In certain embodiments —R⁶ of formula (IX) is —H. In certain embodiments —R⁶ of formula (IX) is —C(O)OH. In certain embodiments —R⁶ of formula (IX) is halogen. In certain embodiments —R⁶ of formula (IX) is —F. In certain embodiments —R⁶ of formula (IX) is —CN. In certain embodiments —R⁶ of formula (IX) is —OH. In certain embodiments —R⁶ of formula (IX) is C₁₋₆ alkyl. In certain embodiments —R⁶ of formula (IX) is C₂₋₆ alkenyl. In certain embodiments —R⁶ of formula (IX) is C₂₋₆ alkynyl. In certain embodiments —R⁶ of formula (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R^{6a} of formula (IX) is selected from the group consisting of —H, —C(O)OH, halogen, —CN, —OH, C₁₋₆ alkyl, C₂₋₆ alkenyl and C₂₋₆ alkynyl. In certain embodiments —R^{6a} of formula (IX) is selected from the group consisting of —H, —C(O)OH, —CN, —OH, C₁₋₆ alkyl, C₂₋₆ alkenyl and C₂₋₆ alkynyl. In certain embodiments —R^{6a} of formula (IX) is selected from the group consisting of —H, —C(O)OH, halogen, —OH, C₁₋₆ alkyl, C₂₋₆ alkenyl and C₂₋₆ alkynyl. In certain embodiments —R^{6a} of formula (IX) is selected from the group consisting of —H, —C(O)OH, —OH and C₁₋₆ alkyl. In certain embodiments —R^{6a} of formula (IX) is —H. In certain embodiments —R^{6a} of formula (IX) is —C(O)OH. In certain embodiments —R^{6a} of formula (IX) is halogen. In certain embodiments —R^{6a} of formula (IX) is —F. In certain embodiments —R^{6a} of formula (IX) is —CN. In certain embodiments —R^{6a} of formula (IX) is —OH. In certain embodiments —R^{6a} of formula (IX) is C₁₋₆ alkyl. In certain embodiments —R^{6a} of formula (IX) is C₂₋₆ alkenyl. In certain embodiments —R^{6a} of formula (IX) is C₂₋₆ alkynyl. In certain embodiments —R^{6a} of formula (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R¹⁰ of formula (IX) is selected from the group consisting of —H, —C(O)OH, halogen, —CN, —OH, C₁₋₆ alkyl, C₂₋₆ alkenyl and C₂₋₆ alkynyl. In certain embodiments —R¹⁰ of formula (IX) is selected from the group consisting of —H, —C(O)OH, —CN, —OH, C₁₋₆ alkyl, C₂₋₆ alkenyl and C₂₋₆ alkynyl. In certain embodiments —R¹⁰ of formula (IX) is selected from the group consisting of —H, —C(O)OH, halogen, —OH, C₁₋₆ alkyl, C₂₋₆ alkenyl and C₂₋₆ alkynyl. In certain embodiments —R¹⁰ of formula (IX) is selected from the group consisting of —H, —C(O)OH, —OH and C₁₋₆ alkyl. In certain embodiments —R¹⁰ of formula (IX) is —H. In certain embodiments —R¹⁰ of formula (IX) is —C(O)OH. In certain embodiments —R¹⁰ of formula (IX) is halogen. In certain embodiments —R¹⁰ of formula (IX) is —F. In certain embodiments —R¹⁰ of formula (IX) is —CN. In certain embodiments —R¹⁰ of formula (IX) is —OH. In certain embodiments —R¹⁰ of formula (IX) is C₁₋₆ alkyl. In certain embodiments —R¹⁰ of formula (IX) is C₂₋₆ alkenyl. In certain embodiments —R¹⁰ of formula (IX) is C₂₋₆ alkynyl. In certain embodiments —R¹⁰ of formula (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R^{10a} of formula (IX) is selected from the group consisting of —H, —C(O)OH, halogen, —CN, —OH, C₁₋₆ alkyl, C₂₋₆ alkenyl and C₂₋₆ alkynyl. In certain embodiments —R^{10a} of formula (IX) is selected from the group consisting of —H, —C(O)OH, —CN, —OH, C₁₋₆ alkyl, C₂₋₆ alkenyl and C₂₋₆ alkynyl. In certain embodiments —R^{10a} of formula (IX) is selected from the group consisting of —H, —C(O)OH, halogen, —OH, C₁₋₆ alkyl, C₂₋₆ alkenyl and C₂₋₆ alkynyl. In certain embodiments —R^{10a} of formula (IX) is selected from the group consisting of —H, —C(O)OH, —OH and C₁₋₆ alkyl. In certain embodiments —R^{10a} of formula (IX) is —H. In certain embodiments —R^{10a} of formula (IX) is —C(O)OH. In certain embodiments —R^{10a} of formula (IX) is halogen. In certain embodiments —R^{10a} of formula (IX) is —F. In certain embodiments —R^{10a} of formula (IX) is —CN. In certain embodiments —R^{10a} of formula (IX) is —OH. In certain embodiments —R^{10a} of formula (IX) is C₁₋₆ alkyl. In certain embodiments —R^{10a} of formula (IX) is C₂₋₆ alkenyl. In certain embodiments —R^{10a} of formula (IX) is C₂₋₆ alkynyl. In certain embodiments —R^{10a} of formula (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R¹¹ of formula (IX) is selected from the group consisting of —H, —C(O)OH, halogen, —CN, —OH, C₁₋₆ alkyl, C₂₋₆ alkenyl and C₂₋₆ alkynyl. In certain embodiments —R¹¹ of formula (IX) is selected from the group consisting of —H, —C(O)OH, —CN, —OH, C₁₋₆ alkyl, C₂₋₆ alkenyl and C₂₋₆ alkynyl. In certain embodiments —R¹¹ of formula (IX) is selected from the group consisting of —H, —C(O)OH, halogen, —OH, C₁₋₆ alkyl, C₂₋₆ alkenyl and C₂₋₆ alkynyl. In certain embodiments —Rⁿ of formula (IX) is selected from the group consisting of —H, —C(O)OH, —OH and C₁₋₆ alkyl. In certain embodiments —Rⁿ of formula (IX) is —H. In certain embodiments —R¹¹ of formula (IX) is —C(O)OH. In certain embodiments —R¹¹ of formula (IX) is halogen. In certain embodiments —R¹¹ of formula (IX) is —F. In certain embodiments —Rⁿ of formula (IX) is —CN. In certain embodiments —Rⁿ of formula (IX) is —OH. In certain embodiments —Rⁿ of formula (IX) is C₁₋₆ alkyl. In certain embodiments —R¹¹ of formula (IX) is C₂₋₆ alkenyl. In certain embodiments —R¹¹ of formula (IX) is C₂₋₆ alkynyl. In certain embodiments —R¹¹ of formula (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R^{11a} of formula (IX) is selected from the group consisting of —H, —C(O)OH, halogen, —CN, —OH, C₁₋₆ alkyl, C₂₋₆ alkenyl and C₂₋₆ alkynyl. In certain embodiments —R^{11a} of formula (IX) is selected from the group consisting of —H, —C(O)OH, —CN, —OH, C₁₋₆ alkyl, C₂₋₆ alkenyl and C₂₋₆ alkynyl. In certain embodiments —R^{11a} of formula (IX) is selected from the group consisting of —H, —C(O)OH, halogen, —OH, C₁₋₆ alkyl, C₂₋₆ alkenyl and C₂₋₆ alkynyl. In certain embodiments —R^{11a} of formula (IX) is selected from the group consisting of —H, —C(O)OH, —OH and C₁₋₆ alkyl. In certain embodiments —R^{11a} of formula (IX) is —H. In certain embodiments —R^{11a} of formula (IX) is —C(O)OH. In certain embodiments —R^{11a} of formula (IX) is halogen. In certain embodiments —R^{11a} of formula (IX) is —F. In certain embodiments —R^{11a} of formula (IX) is —CN. In certain embodiments —R^{11a} of formula (IX) is —OH. In certain embodiments —R^{11a} of formula (IX) is C₁₋₆ alkyl. In certain embodiments —R^{11a} of formula (IX) is C₂₋₆ alkenyl. In certain embodiments —R^{11a} of formula (IX) is C₂₋₆ alkynyl. In certain embodiments —R^{11a} of formula (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R¹² of formula (IX) is selected from the group consisting of —H, —C(O)OH, halogen, —CN, —OH, C₁₋₆ alkyl, C₂₋₆ alkenyl and C₂₋₆ alkynyl. In certain embodiments —R¹² of formula (IX) is selected from the group consisting of —H, —C(O)OH, —CN, —OH, C₁₋₆ alkyl, C₂₋₆ alkenyl and C₂₋₆ alkynyl. In certain embodiments —R¹² of formula (IX) is selected from the group consisting of —H, —C(O)OH, halogen, —OH, C₁₋₆ alkyl, C₂₋₆ alkenyl and C₂₋₆ alkynyl. In certain embodiments —R¹² of formula (IX) is selected from the group consisting of —H, —C(O)OH, —OH and C₁₋₆ alkyl. In certain embodiments —R¹² of formula (IX) is —H. In certain embodiments —R¹² of formula (IX) is —C(O)OH. In certain embodiments —R¹² of formula (IX) is halogen. In certain embodiments —R¹² of formula (IX) is —F. In certain embodiments —R¹² of formula (IX) is —CN. In certain embodiments —R¹² of formula (IX) is —OH. In certain embodiments —R¹² of formula (IX) is C₁₋₆ alkyl. In certain embodiments —R¹² of formula (IX) is C₂₋₆ alkenyl. In certain embodiments —R¹² of formula (IX) is C₂₋₆ alkynyl. In certain embodiments —R¹² of formula (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R^{12a} of formula (IX) is selected from the group consisting of —H, —C(O)OH, halogen, —CN, —OH, C₁₋₆ alkyl, C₂₋₆ alkenyl and C₂₋₆ alkynyl. In certain embodiments —R^{12a} of formula (IX) is selected from the group consisting of —H, —C(O)OH, —CN, —OH, C₁₋₆ alkyl, C₂₋₆ alkenyl and C₂₋₆ alkynyl. In certain embodiments —R^{12a} of formula (IX) is selected from the group consisting of —H, —C(O)OH, halogen, —OH, C₁₋₆ alkyl, C₂₋₆ alkenyl and C₂₋₆ alkynyl. In certain embodiments —R^{12a} of formula (IX) is selected from the group consisting of —H, —C(O)OH, —OH and C₁₋₆ alkyl. In certain embodiments —R^{12a} of formula (IX) is —H. In certain embodiments —R^{12a} of formula (IX) is —C(O)OH. In certain embodiments —R^{12a} of formula (IX) is halogen. In certain embodiments —R^{12a} of formula (IX) is —F. In certain embodiments —R^{12a} of formula (IX) is —CN. In certain embodiments —R^{12a} of formula (IX) is —OH. In certain embodiments —R^{12a} of formula (IX) is C₁₋₆ alkyl. In certain embodiments —R^{12a} of formula (IX) is C₂₋₆ alkenyl. In certain embodiments —R^{12a} of formula (IX) is C₂₋₆ alkynyl. In certain embodiments —R^{12a} of formula (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments each of —R² of formula (IX) is independently selected from the group consisting of —H, —C(O)OH, halogen, —CN, —OH, C₁₋₆ alkyl, C₂₋₆ alkenyl and C₂₋₆ alkynyl. In certain embodiments each of —R² of formula (IX) is independently selected from the group consisting of —H, —C(O)OH, —CN, —OH, C₁₋₆ alkyl, C₂₋₆ alkenyl and C₂₋₆ alkynyl. In certain embodiments each of —R² of formula (IX) is independently selected from the group consisting of —H, —C(O)OH, halogen, —OH, C₁₋₆ alkyl, C₂₋₆ alkenyl and C₂₋₆ alkynyl. In certain embodiments each of —R² of formula (IX) is independently selected from the group consisting of —H, —C(O)OH, —OH and C₁₋₆ alkyl. In certain embodiments each of —R² of formula (IX) is —H. In certain embodiments each of —R² of formula (IX) is —C(O)OH. In certain embodiments each of —R² of formula (IX) is halogen. In certain embodiments each of —R² of formula (IX) is —F. In certain embodiments each of —R² of formula (IX) is —CN. In certain embodiments each of —R² of formula (IX) is —OH. In certain embodiments each of —R² of formula (IX) is C₁₋₆ alkyl. In certain embodiments each of —R² of formula (IX) is C₂₋₆ alkenyl. In certain embodiments each of —R² of formula (IX) is C₂₋₆ alkynyl. In certain embodiments each of —R² of formula (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments each of —R^{2a} of formula (IX) is independently selected from the group consisting of —H, —C(O)OH, halogen, —CN, —OH, C₁₋₆ alkyl, C₂₋₆ alkenyl and C₂₋₆ alkynyl. In certain embodiments each of —R^{2a} of formula (IX) is independently selected from the group consisting of —H, —C(O)OH, —CN, —OH, C₁₋₆ alkyl, C₂₋₆ alkenyl and C₂₋₆ alkynyl. In certain embodiments each of —R^{2a} of formula (IX) is independently selected from the group consisting of —H, —C(O)OH, halogen, —OH, C₁₋₆ alkyl, C₂₋₆ alkenyl and C₂₋₆ alkynyl. In certain embodiments each of —R^{2a} of formula (IX) is independently selected from the group consisting of —H, —C(O)OH, —OH and C₁₋₆ alkyl. In certain embodiments each of —R^{2a} of formula (IX) is —H. In certain embodiments each of —R^{2a} of formula (IX) is —C(O)OH. In certain embodiments each of —R^{2a} of formula (IX) is halogen. In certain embodiments each of —R^{2a} of formula (IX) is —F. In certain embodiments each of —R^{2a} of formula (IX) is —CN. In certain embodiments each of —R^{2a} of formula (IX) is —OH. In certain embodiments each of —R^{2a} of formula (IX) is C₁₋₆ alkyl. In certain embodiments each of —R^{2a} of formula (IX) is C₂₋₆ alkenyl. In certain embodiments each of —R^{2a} of formula (IX) is C₂₋₆ alkynyl. In certain embodiments each of —R^{2a} of formula (IX) is selected from the group consisting of —H, methyl, ethyl, n-propyl, iso-propyl, n-butyl, sec-butyl, iso-butyl, tert-butyl, n-pentyl, 1,1-dimethylpropyl, 2,2-dimethylpropyl, 3-methylbutyl, 1-methylbutyl and 1-ethylpropyl.

In certain embodiments —R³, —R⁴, —R⁵, —R⁷, —R⁸ and —R⁹ of formula (IX) are independently selected from the group consisting of —H, -T, —CN, C₁₋₆ alkyl, C₂₋₆ alkenyl and C₂₋₆ alkynyl. In certain embodiments —R³, —R⁴, —R⁵, —R⁷, —R⁸ and —R⁹ of formula (IX) are independently selected from the group consisting of —H, -T, —CN, C₁₋₆ alkyl and C₂₋₆ alkenyl. In certain embodiments —R³, —R⁴, —R⁵, —R⁷, —R⁸ and —R⁹ of formula (IX) are independently selected from the group consisting of —H, -T, —CN and C₁₋₆ alkyl. In certain embodiments —R³, —R⁴, —R⁵, —R⁷, —R⁸ and —R⁹ of formula (IX) are independently selected from the group consisting of —H, -T and C₁₋₆ alkyl. In certain embodiments —R³, —R⁴, —R⁵, —R⁷, —R⁸ and —R⁹ of formula (IX) are independently selected from the group consisting of —H and C₁₋₆ alkyl.

In certain embodiments —R³ of formula (IX) is selected from the group consisting of —H, -T, —CN, C₁₋₆ alkyl, C₂₋₆ alkenyl and C₂₋₆ alkynyl. In certain embodiments —R³ of formula (IX) is —H. In certain embodiments —R³ of formula (IX) is -T. In certain embodiments —R³ of formula (IX) is —CN. In certain embodiments —R³ of formula (IX) is C₁₋₆ alkyl. In certain embodiments —R³ of formula (IX) is C₂₋₆ alkenyl. In certain embodiments —R³ of formula (IX) is C₂₋₆ alkynyl.

In certain embodiments —R⁴ of formula (IX) is selected from the group consisting of —H, -T, —CN, C₁₋₆ alkyl, C₂₋₆ alkenyl and C₂₋₆ alkynyl. In certain embodiments —R⁴ of formula (IX) is —H. In certain embodiments —R⁴ of formula (IX) is -T. In certain embodiments —R⁴ of formula (IX) is —CN. In certain embodiments —R⁴ of formula (IX) is C₁₋₆ alkyl. In certain embodiments —R⁴ of formula (IX) is C₂₋₆ alkenyl. In certain embodiments —R⁴ of formula (IX) is C₂₋₆ alkynyl.

In certain embodiments —R⁵ of formula (IX) is selected from the group consisting of —H, -T, —CN, C₁₋₆ alkyl, C₂₋₆ alkenyl and C₂₋₆ alkynyl. In certain embodiments —R⁵ of formula (IX) is —H. In certain embodiments —R⁵ of formula (IX) is -T. In certain embodiments —R⁵ of formula (IX) is —CN. In certain embodiments —R⁵ of formula (IX) is C₁₋₆ alkyl. In certain embodiments —R⁵ of formula (IX) is C₂₋₆ alkenyl. In certain embodiments —R⁵ of formula (IX) is C₂₋₆ alkynyl.

In certain embodiments —R⁷ of formula (IX) is selected from the group consisting of —H, -T, —CN, C₁₋₆ alkyl, C₂₋₆ alkenyl and C₂₋₆ alkynyl. In certain embodiments —R⁷ of formula (IX) is —H. In certain embodiments —R⁷ of formula (IX) is -T. In certain embodiments —R⁷ of formula (IX) is —CN. In certain embodiments —R⁷ of formula (IX) is C₁₋₆ alkyl. In certain embodiments —R⁷ of formula (IX) is C₂₋₆ alkenyl. In certain embodiments —R⁷ of formula (IX) is C₂₋₆ alkynyl.

In certain embodiments —R⁸ of formula (IX) is selected from the group consisting of —H, -T, —CN, C₁₋₆ alkyl, C₂₋₆ alkenyl and C₂₋₆ alkynyl. In certain embodiments —R⁸ of formula (IX) is —H. In certain embodiments —R⁸ of formula (IX) is -T. In certain embodiments —R⁸ of formula (IX) is —CN. In certain embodiments —R⁸ of formula (IX) is C₁₋₆ alkyl. In certain embodiments —R⁸ of formula (IX) is C₂₋₆ alkenyl. In certain embodiments —R⁸ of formula (IX) is C₂₋₆ alkynyl.

In certain embodiments —R⁹ of formula (IX) is selected from the group consisting of —H, -T, —CN, C₁₋₆ alkyl, C₂₋₆ alkenyl and C₂₋₆ alkynyl. In certain embodiments —R⁹ of formula (IX) is —H. In certain embodiments —R⁹ of formula (IX) is -T. In certain embodiments —R⁹ of formula (IX) is —CN. In certain embodiments —R⁹ of formula (IX) is C₁₋₆ alkyl. In certain embodiments —R⁹ of formula (IX) is C₂₋₆ alkenyl. In certain embodiments —R⁹ of formula (IX) is C₂₋₆ alkynyl.

In certain embodiments T of formula (IX) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments T of formula (IX) is phenyl. In certain embodiments T of formula (IX) is naphthyl. In certain embodiments T of formula (IX) is indenyl. In certain embodiments T of formula (IX) is indanyl. In certain embodiments T of formula (IX) is tetralinyl. In certain embodiments T of formula (IX) is C₃₋₁₀ cycloalkyl.

In certain embodiments T of formula (IX) is 3- to 10-membered heterocyclyl. In certain embodiments T of formula (IX) is 8- to 11-membered heterobicyclyl.

In certain embodiments T of formula (IX) is substituted with one or more —R¹³, which are the same or different.

In certain embodiments T of formula (IX) is substituted with one —R¹³.

In certain embodiments T of formula (IX) is not substituted with —R¹³.

In certain embodiments —R¹³ of formula (IX) is selected from the group consisting of —H, —NO₂, —OCH₃, —CN, —N(R¹⁴)(R^{14a}), —OH, —C(O)OH and C₁₋₆ alkyl.

In certain embodiments —R¹³ of formula (IX) is —H. In certain embodiments —R¹³ of formula (IX) is —NO₂. In certain embodiments —R¹³ of formula (IX) is —OCH₃. In certain embodiments —R¹³ of formula (IX) is —CN. In certain embodiments —R¹³ of formula (IX) is —N(R¹⁴)(R^{14a}). In certain embodiments —R¹³ of formula (IX) is —OH. In certain embodiments —R¹³ of formula (IX) is —C(O)OH. In certain embodiments —R¹³ of formula (IX) is C₁₋₆ alkyl.

In certain embodiments —R¹⁴ and —R^{14a} of formula (IX) are independently selected from the group consisting of —H and C₁₋₆ alkyl. In certain embodiments —R¹⁴ of formula (IX) is —H. In certain embodiments —R¹⁴ of formula (IX) is C₁₋₆ alkyl. In certain embodiments —R^{14a} of formula (IX) is —H. In certain embodiments —R^{14a} of formula (IX) is C₁₋₆ alkyl.

In certain embodiments n of formula (IX) is selected from the group consisting of 0, 1, 2 and 3. In certain embodiments n of formula (IX) is selected from the group consisting of 0, 1 and 2. In certain embodiments n of formula (IX) is selected from the group consisting of 0 and 1. In certain embodiments n of formula (IX) is 0. In certain embodiments n of formula (I) is 1. In certain embodiments n of formula (IX) is 2. In certain embodiments n of formula (I) is 3. In certain embodiments n of formula (IX) is 4.

In certain embodiments -L¹- of formula (IX) is connected to -D through a linkage selected from the group consisting of amide, carbamate, dithiocarbamate, O-thiocarbamate, S-thiocarbamate, urea, thiourea, thioamide, amidine and guanidine. It is understood that some of these linkages may not be reversible per se, but that in the present invention neighboring groups present in -L¹-, such as for example amide, primary amine, secondary amine and tertiary amine, render these linkages reversible.

In certain embodiments -L¹- of formula (XI) is conjugated to -D through an amide linkage, i.e. ═X^{J} is ═O and —X²— is —C(R⁶)(R^{6a})—.

In certain embodiments -L¹- of formula (IX) is conjugated to -D through a carbamate linkage, i.e. ═X¹ is ═O and —X²— is —O—.

In certain embodiments -L¹- of formula (IX) is conjugated to -D through a dithiocarbamate linkage, i.e. ═X is ═S and —X²— is —S—.

In certain embodiments -L¹- of formula (IX) is conjugated to -D through an O-thiocarbamate linkage, i.e. ═X¹ is ═S and —X²— is —O—.

In certain embodiments -L¹- of formula (IX) is conjugated to -D through a S-thiocarbamate linkage, i.e. ═X¹ is ═O and —X²— is —S—.

In certain embodiments -L¹- of formula (IX) is conjugated to -D through a urea linkage, i.e. ═X¹ is ═O and —X²— is —N(R⁵)—.

In certain embodiments -L¹- of formula (IX) is conjugated to -D through a thiourea linkage, i.e. ═X¹ is ═S and —X²— is —N(R⁵)—.

In certain embodiments -L¹- of formula (IX) is conjugated to -D through a thioamide linkage, i.e. ═X¹ is ═S and —X²— is —C(R⁶)(R^{6a})—. In certain embodiments -L¹- of formula (IX) is conjugated to -D through an amidine linkage, i.e. ═X¹ is ═N(R⁴) and —X²— is —C(R⁶)(R^{6a})—.

In certain embodiments -L¹- of formula (IX) is conjugated to -D through a guanidine linkage, i.e. ═X¹ is ═N(R⁴) and —X²— is —N(R⁵)—.

In certain embodiments -L¹- is of formula (IX′):

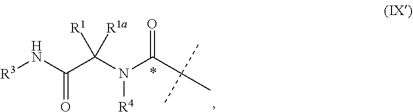

wherein the dashed line indicates the attachment to a π-electron-pair-donating heteroaromatic N of -D; and
—R¹, —R^{1a}, —R³ and —R⁴ are used as defined in formula (IX).

In certain embodiments —R¹ and —R^{1a} of formula (IX′) are both —H.

In certain embodiments —R¹ of formula (IX′) is —H and —R^{1a} of formula (IX′) is C₁₋₆ alkyl.

In certain embodiments —R³ of formula (IX′) is C₁₋₆ alkyl.

In certain embodiments —R⁴ of formula (IX′) is methyl.

In certain embodiments —R⁴ of formula (IX′) is ethyl.

In certain embodiments -L¹- is of formula (X)

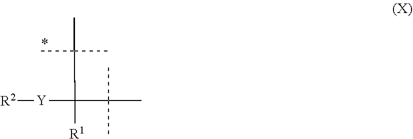

wherein
the dashed line marked with an asterisk indicates the attachment to -L
the unmarked dashed line indicates the attachment to a π-electron-pair-donating heteroaromatic N of -D;
—Y— is selected from the group consisting of —N(R³)—, —O— and —S—;
—R¹, —R² and —R³ are independently selected from the group consisting of —H, -T, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R⁴, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁵)—, —S(O)₂N(R⁵)—, —S(O)N(R⁵)—, —S(O)₂—, —S(O)—, —N(R⁵)S(O)₂N(R^{5a})—, —S—, —N(R⁵), —OC(OR⁵)(R^{5a})—, —N(R⁵)C(O)N(R^{5a})— and —OC(O)N(R⁵)—;
   each T is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each T is independently optionally substituted with one or more —R⁴, which are the same or different;
   wherein —R⁴, —R⁵ and —R^{5a} are independently selected from the group consisting of —H and C₁₋₆ alkyl; wherein C₁₋₆ alkyl is optionally substituted with one or more halogen, which are the same or different; and
wherein -L¹- is substituted with -L²- and wherein -L¹- is optionally further substituted.

The optional further substituents of -L¹- of formula (X) are as described elsewhere herein.

In certain embodiments -L¹- of formula (X) is not further substituted.

In certain embodiments —Y— of formula (X) is —N(R³)—.

In certain embodiments —Y— of formula (X) is —O—.

In certain embodiments —Y— of formula (X) is —S—.

In certain embodiments —R¹, —R² and —R³ of formula (X) are independently selected from the group consisting of —H, -T, C₁₋₆ alkyl, C₂₋₆ alkenyl and C₂₋₆ alkynyl.

In certain embodiments —R¹ of formula (X) is independently selected from the group consisting of —H, -T, C₁₋₆ alkyl, C₂₋₆ alkenyl and C₂₋₆ alkynyl. In certain embodiments —R¹ of formula (X) is —H. In certain embodiments —R¹ of formula (X) is -T. In certain embodiments —R¹ of formula (X) is C₁₋₆ alkyl. In certain embodiments —R¹ of formula (X) is C₂₋₆ alkenyl. In certain embodiments —R¹ of formula (X) is C₂₋₆ alkynyl.

In certain embodiments —R² of formula (X) is independently selected from the group consisting of —H, -T, C₁₋₆ alkyl, C₂₋₆ alkenyl and C₂₋₆ alkynyl. In certain embodiments —R² of formula (X) is —H. In certain embodiments —R² of formula (X) is -T. In certain embodiments —R² of formula (X) is C₁₋₆ alkyl. In certain embodiments —R² of formula (X) is C₂₋₆ alkenyl. In certain embodiments —R² of formula (X) is C₂₋₆ alkynyl.

In certain embodiments —R³ of formula (X) is independently selected from the group consisting of —H, -T, C₁₋₆ alkyl, C₂₋₆ alkenyl and C₂₋₆ alkynyl. In certain embodiments —R³ of formula (X) is —H. In certain embodiments —R³ of formula (X) is -T. In certain embodiments —R³ of formula (X) is C₁₋₆ alkyl. In certain embodiments —R³ of formula (X) is C₂₋₆ alkenyl. In certain embodiments —R³ of formula (X) is C₂₋₆ alkynyl.

In certain embodiments T of formula (X) is selected from the group consisting of phenyl, naphthyl, indenyl, indanyl, tetralinyl, C₃₋₁₀ cycloalkyl, 3- to 10-membered heterocyclyl and 8- to 11- heterobicyclyl. In certain embodiments T of formula (X) is phenyl. In certain embodiments T of formula (X) is naphthyl. In certain embodiments T of formula (X) is indenyl. In certain embodiments T of formula (X) is indanyl. In certain embodiments T of formula (X) is tetralinyl. In certain embodiments T of formula (X) is C₃₋₁₀ cycloalkyl. In certain embodiments T of formula (X) is 3- to 10-membered heterocyclyl. In certain embodiments T of formula (X) is 8- to 11-heterobicyclyl.

In certain embodiments T of formula (X) is substituted with one or more —R⁴.

In certain embodiments T of formula (X) is substituted with one —R⁴.

In certain embodiments T of formula (X) is not substituted with —R⁴.

In certain embodiments —R⁴, —R⁵ and —R^{5a} of formula (X) are independently selected from the group consisting of —H and C₁₋₆ alkyl.

In certain embodiments —R⁴ of formula (X) is selected from the group consisting of —H and C₁₋₆ alkyl. In certain embodiments —R⁴ of formula (X) is —H. In certain embodiments —R⁴ of formula (X) is C₁₋₆ alkyl.

In certain embodiments —R⁵ of formula (X) is selected from the group consisting of —H and C₁₋₆ alkyl. In certain embodiments —R⁵ of formula (X) is —H. In certain embodiments —R⁵ of formula (X) is C₁₋₆ alkyl.

In certain embodiments —R^{5a} of formula (X) is selected from the group consisting of —H and C₁₋₆ alkyl. In certain embodiments —R^{5a} of formula (X) is —H. In certain embodiments —R^{5a} of formula (X) is C₁₋₆ alkyl.

In certain embodiments -L¹- of formula (X) is connected to -D through a heminal linkage.

In certain embodiments -L¹- of formula (X) is connected to -D through an aminal linkage.

In certain embodiments -L¹- of formula (X) is connected to -D through a hemithioaminal linkage.

A moiety -L¹- suitable for drugs D that when bound to -L¹- comprise an electron-donating heteroaromatic N⁺ moiety or a quaternary ammonium cation and becomes a moiety -D⁺ upon linkage with -L¹- is of formula (XI)

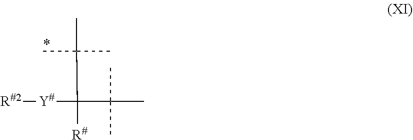

wherein
the dashed line marked with an asterisk indicates the attachment to -L²-, the unmarked dashed line indicates the attachment to the N⁺ of -D⁺;
—Y^{#}— is selected from the group consisting of —N(R^{#3})—, —O— and —S—;
—R^{#1}, —R^{#2} and —R^{#3} are independently selected from the group consisting of —H, -T^{#}, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R^{#4}, which are the same or different; and wherein C₁₋₆ alkyl, C₂₋₆ alkenyl and C₂₋₆ alkynyl are optionally interrupted by one or more groups selected from the group consisting of -T^{#}-, —C(O)O—, —O—, —C(O)—, —C(O)N(R^{#5})—, —S(O)₂N(R^{#5})—, —S(O)N(R^{#5})—, —S(O)₂₋, —S(O)—, —N(R^{#5})S(O)₂N(R^{#5a})—, —S—, —N(R^{#5}), —OC(OR^{#5})(R^{#5a})—, —N(R^{#5})C(O)N(R^{#5a})— and —OC(O)N(R^{#5})—;
   each T^{#} is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each T^{#} is independently optionally substituted with one or more —R^{#4}, which are the same or different; and
   wherein —R^{#4}, —R^{#5} and —R^{#5a} are independently selected from the group consisting of —H and C₁₋₆ alkyl; wherein C₁₋₆ alkyl is optionally substituted with one or more halogen, which are the same or different; and
each -L¹- is substituted with -L²- and optionally further substituted.

It is understood that in certain embodiments -D⁺ may comprise both an electron-donating heteroaromatic N⁺ and a quaternary ammonium cation and analogously the corresponding D may comprise both an electron-donating heteroaromatic N and a tertiary amine. It is also understood that if D is conjugated to -L¹-, then -D⁺ and -L¹- form a quaternary ammonium cation, for which there may be a counter anion. Examples of counter anions include, but are not limited to, chloride, bromide, acetate, bicarbonate, sulfate, bisulfate, nitrate, carbonate, alkyl sulfonate, aryl sulfonate and phosphate.

Such drug moiety -D⁺ comprises at least one, such as one, two, three, four, five, six, seven, eight, nine or ten electron-donating heteroaromatic N⁺ or quaternary ammonium cations and analogously the corresponding released drug D comprises at least one, such as one, two, three, four, five, six, seven, eight, nine or ten electron-donating heteroaromatic N or tertiary amines. Examples of chemical structures including heteroaromatic nitrogens i.e. N⁺ or N, that donate an electron to the aromatic π-system include, but are not limited to, pyridine, pyridazine, pyrimidine, quinoline, quinazoline, quinoxaline, pyrazole, imidazole, isoindazole, indazole, purine, tetrazole, triazole and triazine. For example, in the imidazole ring below the heteroaromatic nitrogen which donates one electron to the aromatic π-system is marked with “§”:

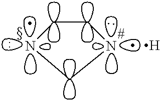

Such electron-donating heteroaromatic nitrogen atoms do not comprise heteroaromatic nitrogen atoms which donate one electron pair (i.e. not one electron) to the aromatic n-system, such as for example the nitrogen that is marked with “#” in the abovementioned imidazole ring structure. The drug D may exist in one or more tautomeric forms, such as with one hydrogen atom moving between at least two heteroaromatic nitrogen atoms. In all such cases, the linker moiety is covalently and reversibly attached at a heteroaromatic nitrogen that donates an electron to the aromatic π-system.

In certain embodiments —Y^{#}— of formula (XI) is —N(R^{#3})—. In certain embodiments —Y^{#}— of formula (XI) is —O—. In certain embodiments —Y^{#}— of formula (XI) is —S—.

In certain embodiments —R^{#1}, —R^{#2} and —R^{#3} of formula (XI) are independently selected from the group consisting of —H, -T^{#}, C₁₋₆ alkyl, C₂₋₆ alkenyl and C₂₋₆ alkynyl.

In certain embodiments —R^{#1} of formula (XI) is independently selected from the group consisting of —H, -T^{#}, C₁₋₆ alkyl, C₂₋₆ alkenyl and C₂₋₆ alkynyl. In certain embodiments —R^{#1} of formula (XI) is —H. In certain embodiments —R^{#1} of formula (XI) is -T^{#}. In certain embodiments —R^{#1} of formula (XI) is C₁₋₆ alkyl. In certain embodiments —R^{#1} of formula (XI) is C₂₋₆ alkenyl. In certain embodiments —R^{#1} of formula (XI) is C₂₋₆ alkynyl.

In certain embodiments —R^{#2} of formula (XI) is independently selected from the group consisting of —H, -T^{#}, C₁₋₆ alkyl, C₂₋₆ alkenyl and C₂₋₆ alkynyl. In certain embodiments —R^{#2} of formula (XI) is —H. In certain embodiments —R² of formula (XI) is -T^{#}. In certain embodiments —R^{#2} of formula (XI) is C₁₋₆ alkyl. In certain embodiments —R^{#2} of formula (XI) is C₂₋₆ alkenyl. In certain embodiments —R^{#2} of formula (XI) is C₂₋₆ alkynyl.

In certain embodiments, —R^{#3} of formula (XI) is independently selected from the group consisting of —H, -T^{#}, C₁₋₆ alkyl, C₂₋₆ alkenyl and C₂₋₆ alkynyl. In certain embodiments —R^{#3} of formula (XI) is —H. In certain embodiments —R^{#3} of formula (XI) is -T^{#}. In certain embodiments, —R^{#3} is C₁₋₆ alkyl. In certain embodiments —R^{#3} of formula (XI) is C₂₋₆ alkenyl. In certain embodiments —R^{#3} of formula (XI) is C₂₋₆ alkynyl.

In certain embodiments T^{#} of formula (XI) is selected from the group consisting of phenyl, naphthyl, indenyl, indanyl, tetralinyl, C₃₋₁₀ cycloalkyl, 3- to 10-membered heterocyclyl and 8- to 11- heterobicyclyl. In certain embodiments T^{#} of formula (XI) is phenyl. In certain embodiments T^{#} of formula (XI) is naphthyl. In certain embodiments T^{#} of formula (XI) is indenyl. In certain embodiments T^{#} of formula (XI) is indanyl. In certain embodiments T^{#} of formula (XI) is tetralinyl. In certain embodiments T^{#} of formula (XI) is C₃₋₁₀ cycloalkyl. In certain embodiments T^{#} of formula (XI) is 3- to 10-membered heterocyclyl. In certain embodiments T^{#} of formula (XI) is 8- to 11-heterobicyclyl. In certain embodiments T^{#} of formula (XI) is substituted with one or more —R⁴.

In certain embodiments T^{#} of formula (XI) is substituted with one —R⁴.

In certain embodiments T^{#} of formula (XI) is not substituted with —R⁴.

In certain embodiments —R^{#4}, —R^{#5} and —R^{#5a} of formula (XI) are independently selected from the group consisting of —H and C₁₋₆ alkyl.

In certain embodiments —R^{#4} of formula (XI) is selected from the group consisting of —H and C₁₋₆ alkyl. In certain embodiments —R^{#4} of formula (XI) is —H. In certain embodiments —R^{#4} of formula (XI) is C₁₋₆ alkyl.

In certain embodiments —R^{#5} of formula (XI) is selected from the group consisting of —H and C₁₋₆ alkyl. In certain embodiments —R⁵ of formula (XI) is —H. In certain embodiments —R^{#5} of formula (XI) is C₁₋₆ alkyl.

In certain embodiments —R^{#5a} of formula (XI) is selected from the group consisting of —H and C₁₋₆ alkyl. In certain embodiments —R^{#5a} of formula (XI) is —H. In certain embodiments —R^{#5a} of formula (XI) is C₁₋₆ alkyl.

A moiety -L¹- suitable for drugs D that when bound to -L¹- comprise an electron-donating heteroaromatic N⁺ moiety or a quaternary ammonium cation and becomes a moiety -D⁺ upon linkage with -L¹- is of formula (XII)

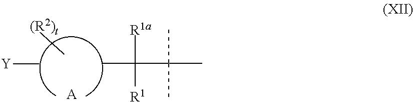

wherein
the dashed line indicates the attachment to the N⁺ of -D⁺;
t is selected from the group consisting of 0, 1, 2, 3, 4, 5 and 6;
-A- is a ring selected from the group consisting of monocyclic or bicyclic aryl and heteroaryl, provided that -A- is connected to —Y and —C(R¹)(R^{1a})— via carbon atoms; wherein said monocyclic or bicyclic aryl and heteroaryl are optionally substituted with one or more —R², which are the same or different;
—R¹, —R^{1a} and each —R² are independently selected from the group consisting of —H, —C(O)OH, -halogen, —NO₂, —CN, —OH, C₁₋₆ alkyl, C₂₋₆ alkenyl and C₂₋₆ alkynyl; wherein C₁₋₆ alkyl, C₂₋₆ alkenyl and C₂₋₆ alkynyl are optionally substituted with one or more —R³, which are the same or different; and wherein C₁₋₆ alkyl, C₂₋₆ alkenyl and C₂₋₆ alkynyl are optionally interrupted by one or more groups selected from the group consisting of -T-, —C(O)O—, —O—, —C(O)—, —C(O)N(R⁴)—, —S(O)₂N(R⁴)—, —S(O)N(R⁴)—, —S(O)₂—, —S(O)—, —N(R⁴)S(O)₂N(R^{4a})—, —S—, —N(R⁴)—, —OC(OR⁴)(R^{4a})—, —N(R⁴)C(O)N(R^{4a})— and —OC(O)N(R⁴)—;
   each -T- is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each -T- is independently optionally substituted with one or more —R³, which are the same or different;
   wherein —R³ is selected from the group consisting of —H, —NO₂, —OCH₃, —CN, —N(R⁴)(R^{4a}), —OH, —C(O)OH and C₁₋₆ alkyl; wherein C₁₋₆ alkyl is optionally substituted with one or more halogen, which are the same or different;
   wherein —R⁴ and —R^{4a} are independently selected from the group consisting of —H and C₁₋₆ alkyl; wherein C₁₋₆ alkyl is optionally substituted with one or more halogen, which are the same or different;
—Y is selected from the group consisting of:

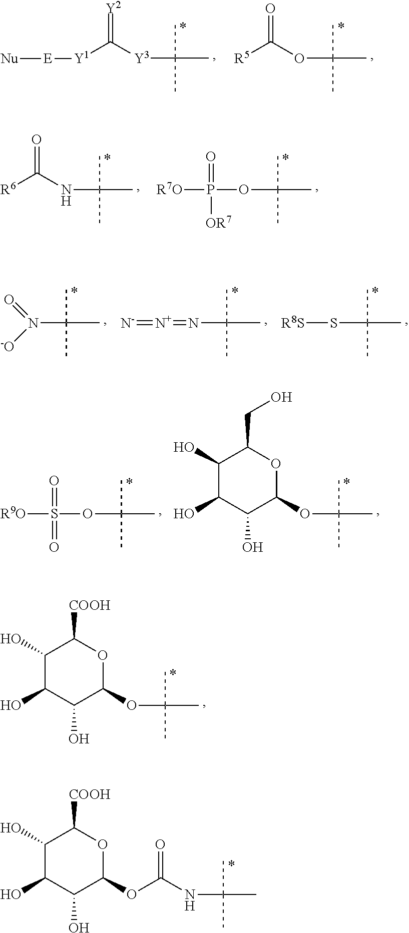

and a peptidyl moiety;
wherein
the dashed line marked with an asterisk indicates the attachment to -A-;
-Nu is a nucleophile;
Y¹— is selected from the group consisting of —O—, —C(R¹⁰)(R^{10a})—, —N(R¹¹)— and —S—;
═Y² is selected from the group consisting of ═O, ═S and ═N(R¹²);
—Y³— is selected from the group consisting of —O—, —S— and —N(R¹³);
-E- is selected from the group consisting of C₁₋₆ alkyl, C₂₋₆ alkenyl, C₂₋₆ alkynyl and -Q-; wherein C₁₋₆ alkyl, C₂₋₆ alkenyl, C₂₋₆ alkynyl are optionally substituted with one or more —R¹⁴, which are the same or different;
—R⁵, —R⁶, each —R⁷, —R⁸, —R⁹, —R¹⁰, —R^{10a}, —R¹¹, —R¹² and —R¹³ are independently selected from the group consisting of C₁₋₂₀ alkyl, C₂₋₂₀ alkenyl, C₂₋₂₀ alkynyl and -Q; wherein C₁₋₂₀ alkyl, C₂₋₂₀ alkenyl and C₂₋₂₀ alkynyl are optionally substituted with one or more —R¹⁴, which are the same or different; and wherein C₁₋₁₀ alkyl, C₂₋₁₀ alkenyl and C₂₋₁₀ alkynyl are optionally interrupted by one or more groups selected from the group consisting of Q, —C(O)O—, —O—, —C(O)—, —C(O)N(R¹⁵)—, —S(O)₂N(R¹⁵), —S(O)N(R¹⁵)—, —S(O)₂—, —S(O)—, —N(R¹⁵)S(O)₂N(R^{15a})—, —S—, —N(R¹⁵)—, —OC(OR¹⁵)R^{15a}—, —N(R¹⁵)C(O)N(R^{15a})— and —OC(O)N(R¹⁵)—;
   each Q is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q is independently optionally substituted with one or more —R¹⁴, which are the same or different;
   wherein —R¹⁴, —R¹⁵ and —R^{15a} are independently selected from the group consisting of —H and C₁₋₆ alkyl; wherein C₁₋₆ alkyl is optionally substituted with one or more halogen, which are the same or different; and
each -L¹- is substituted with -L²- and optionally further substituted.

It is understood that in certain embodiments -D⁺ may comprise both an electron-donating heteroaromatic N⁺ and a quaternary ammonium cation and analogously the corresponding D may comprise both an electron-donating heteroaromatic N and a tertiary amine. It is also understood that if D is conjugated to -L¹-, then -D⁺ and -L¹- form a quaternary ammonium cation, for which there may be a counter anion. Examples of counter anions include, but are not limited to, chloride, bromide, acetate, bicarbonate, sulfate, bisulfate, nitrate, carbonate, alkyl sulfonate, aryl sulfonate and phosphate.

The optional further substituents of -L¹- of formula (XII) are as described elsewhere herein.

In certain embodiments -L¹- of formula (XII) is not further substituted.

Such drug moiety -D⁺ comprises at least one, such as one, two, three, four, five, six, seven, eight, nine or ten electron-donating heteroaromatic N⁺ or quaternary ammonium cations and analogously the corresponding released drug D comprises at least one, such as one, two, three, four, five, six, seven, eight, nine or ten electron-donating heteroaromatic N or tertiary amines. Examples of chemical structures including heteroaromatic nitrogens i.e. N⁺ or N, that donate an electron to the aromatic π-system include, but are not limited to, pyridine, pyridazine, pyrimidine, quinoline, quinazoline, quinoxaline, pyrazole, imidazole, isoindazole, indazole, purine, tetrazole, triazole and triazine. For example, in the imidazole ring below the heteroaromatic nitrogen which donates one electron to the aromatic 7T-system is marked with “§”:

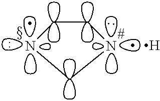

Such electron-donating heteroaromatic nitrogen atoms do not comprise heteroaromatic nitrogen atoms which donate one electron pair (i.e. not one electron) to the aromatic n-system, such as for example the nitrogen that is marked with “#” in the abovementioned imidazole ring structure. The drug D may exist in one or more tautomeric forms, such as with one hydrogen atom moving between at least two heteroaromatic nitrogen atoms. In all such cases, the linker moiety is covalently and reversibly attached at a heteroaromatic nitrogen that donates an electron to the aromatic π-system.

As used herein, the term “monocyclic or bicyclic aryl” means an aromatic hydrocarbon ring system which may be monocyclic or bicyclic, wherein the monocyclic aryl ring consists of at least 5 ring carbon atoms and may comprise up to 10 ring carbon atoms and wherein the bicylic aryl ring consists of at least 8 ring carbon atoms and may comprise up to 12 ring carbon atoms. Each hydrogen atom of a monocyclic or bicyclic aryl may be replaced by a substituent as defined below.

As used herein, the term “monocyclic or bicyclic heteroaryl” means a monocyclic aromatic ring system that may comprise 2 to 6 ring carbon atoms and 1 to 3 ring heteroatoms or a bicyclic aromatic ring system that may comprise 3 to 9 ring carbon atoms and 1 to 5 ring heteroatoms, such as nitrogen, oxygen and sulfur. Examples for monocyclic or bicyclic heteroaryl groups include, but are not limited to, benzofuranyl, benzothiophenyl, furanyl, imidazolyl, indolyl, azaindolyl, azabenzimidazolyl, benzoxazolyl, benzthiazolyl, benzthiadiazolyl, benzotriazolyl, tetrazinyl, tetrazolyl, isothiazolyl, oxazolyl, isoxazolyl, pyrazinyl, pyrazolyl, pyridazinyl, pyridinyl, pyrimidinyl, pyrrolyl, quinolinyl, quinazolinyl, quinoxalinyl, triazolyl, thiazolyl and thiophenyl. Each hydrogen atom of a monocyclic or bicyclic heteroaryl may be replaced by a substituent as defined below.

As used herein, the term “nucleophile” refers to a reagent or functional group that forms a bond to its reaction partner, i.e. the electrophile by donating both bonding electrons.

In certain embodiments t of formula (XII) is 0. In certain embodiments t of formula (XII) is 1. In certain embodiments t of formula (XII) is 2. In certain embodiments t of formula (XII) is 3. In certain embodiments t of formula (XII) is 4. In certain embodiments t of formula (XII) is 5. In certain embodiments t of formula (XII) is 6.

In certain embodiments -A- of formula (XII) is a ring selected from the group consisting of monocyclic or bicyclic aryl and heteroaryl. In certain embodiments -A- of formula (XII) is substituted with one or more —R² which are the same or different. In certain embodiments -A- of formula (XII) is not substituted with —R². In certain embodiments -A- of formula (XII) is selected from the group consisting of:

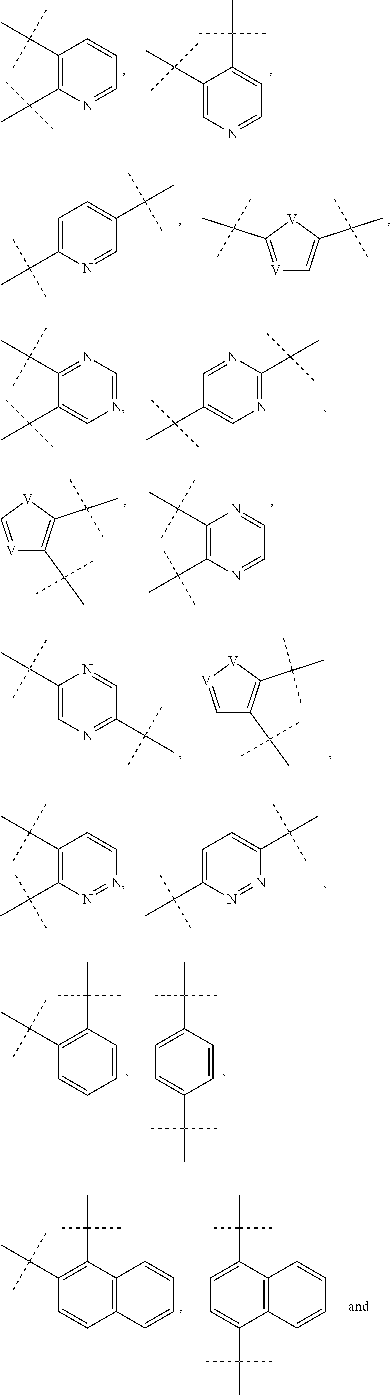

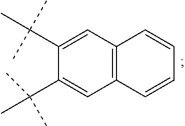

wherein each V is independently selected from the group consisting of O, S and N.

In certain embodiments —R¹, —R^{1a} and each —R² of formula (XII) are independently selected from the group consisting of —H, —C(O)OH, -halogen, —CN, —NO₂, —OH, C₁₋₆ alkyl, C₂₋₆ alkenyl and C₂₋₆ alkynyl. In certain embodiments —R¹ of formula (XII) is —H. In certain embodiments —R¹ of formula (XII) is —C(O)OH. In certain embodiments —R¹ of formula (XII) is -halogen. In certain embodiments —R¹ of formula (XII) is —F. In certain embodiments —R¹ of formula (XII) is —CN. In certain embodiments —R¹ of formula (XII) is —NO₂. In certain embodiments —R¹ of formula (XII) is —OH. In certain embodiments —R¹ of formula (XII) is C₁₋₆ alkyl. In certain embodiments —R¹ of formula (XII) is C₂₋₆ alkenyl. In certain embodiments —R¹ is C₂₋₆ alkynyl. In certain embodiments —R^{1a} of formula (XII) is —H. In certain embodiments —R^{1a} of formula (XII) is —C(O)OH. In certain embodiments —R^{1a} of formula (XII) is -halogen. In certain embodiments —R^{1a} of formula (XII) is —F. In certain embodiments —R^{1a} of formula (XII) is —CN. In certain embodiments —R^{1a} of formula (XII) is —NO₂. In certain embodiments —R^{1a} of formula (XII) is —OH. In certain embodiments —R^{1a} of formula (XII) is C₁₋₆ alkyl. In certain embodiments —R^{1a} of formula (XII) is C₂₋₆ alkenyl. In certain embodiments —R^{1a} of formula (XII) is C₂₋₆ alkynyl.

In certain embodiments each of —R² of formula (XII) is independently selected from the group consisting of —H, —C(O)OH, -halogen, —CN, —NO₂, —OH, C₁₋₆ alkyl, C₂₋₆ alkenyl and C₂₋₆ alkynyl. In certain embodiments each of —R² of formula (XII) is —H. In certain embodiments each of —R² of formula (XII) is —C(O)OH. In certain embodiments each of —R² of formula (XII) is -halogen. In certain embodiments each of —R² of formula (XII) is —F. In certain embodiments each of —R² of formula (XII) is —CN. In certain embodiments each of —R² of formula (XII) is —NO₂. In certain embodiments each of —R² of formula (XII) is —OH. In certain embodiments each of —R² of formula (XII) is C₁₋₆ alkyl. In certain embodiments each of —R² of formula (XII) is C₂₋₆ alkenyl. In certain embodiments each of —R² of formula (XII) is C₂₋₆ alkynyl.

In certain embodiments T of formula (XII) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments T of formula (XII) is phenyl. In certain embodiments T of formula (XII) is naphthyl. In certain embodiments T of formula (XII) is indenyl. In certain embodiments T of formula (XII) is indanyl. In certain embodiments T of formula (XII) is tetralinyl. In certain embodiments T of formula (XII) is C₃₋₁₀ cycloalkyl. In certain embodiments T of formula (XII) is 3- to 10-membered heterocyclyl. In certain embodiments T of formula (XII) is 8- to 11-membered heterobicyclyl.

In certain embodiments T of formula (XII) is substituted with one or more —R³, which are the same or different. In certain embodiments T of formula (XII) is substituted with one —R³. In certain embodiments T of formula (XII) is not substituted with —R³.

In certain embodiments —R³ of formula (XII) is selected from the group consisting of —H, —NO₂, —OCH₃, —CN, —N(R⁴)(R^{4a}), —OH, —C(O)OH and C₁₋₆ alkyl. In certain embodiments —R³ of formula (XII) is —H. In certain embodiments —R³ of formula (XII) is —NO₂. In certain embodiments —R³ of formula (XII) is —OCH₃. In certain embodiments —R³ of formula (XII) is —CN. In certain embodiments —R³ of formula (XII) is —N(R⁴)(R^{4a}). In certain embodiments —R³ of formula (XII) is —OH. In certain embodiments —R³ of formula (XII) is —C(O)OH. In certain embodiments —R³ of formula (XII) is C₁₋₆ alkyl. In certain embodiments —R⁴ and —R^{4a} of formula (XII) are independently selected from the group consisting of —H and C₁₋₆ alkyl. In certain embodiments —R⁴ of formula (XII) is —H. In certain embodiments —R⁴ is C₁₋₆ alkyl. In certain embodiments —R^{4a} of formula (XII) is —H. In certain embodiments —R^{4a} of formula (XII) is C₁₋₆ alkyl.

In certain embodiments —Y of formula (XII) is

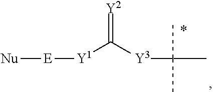

wherein -Nu, -E, —Y¹—, ═Y² and —Y³— are as defined elsewhere herein and the dashed line marked with an asterisk indicates the attachment to -A- of formula (XII).

In certain embodiments -Nu of formula (XII) is a nucleophile selected from the group consisting of primary, secondary, tertiary amine and amide. In certain embodiments -Nu of formula (XII) is a primary amine. In certain embodiments -Nu of formula (XII) is a secondary amine. In certain embodiments -Nu of formula (XII) is a tertiary amine. In certain embodiments -Nu of formula (XII) is an amide.

In certain embodiments —Y¹— of formula (XII) is selected from the group consisting of —O—, —C(R¹⁰)(R^{10a})—, —N(R¹¹)- and —S—. In certain embodiments —Y¹— of formula (XII) is —O—. In certain embodiments —Y¹— of formula (XII) is —C(R¹⁰)(R^{10a})—. In certain embodiments —Y¹— of formula (XII) is —N(R¹¹)—. In certain embodiments —Y¹— is —S—.

In certain embodiments ═Y² of formula (XII) is selected from the group consisting of ═O, ═S and ═N(R¹²). In certain embodiments ═Y² of formula (XII) is ═O. In certain embodiments ═Y² of formula (XII) is ═S. In certain embodiments ═Y² of formula (XII) is ═N(R¹²).

In certain embodiments —Y³— of formula (XII) is selected from the group consisting of —O—, —S— and —N(R¹³). In certain embodiments —Y³— of formula (XII) is —O—. In certain embodiments —Y³— of formula (XII) is —S—. In certain embodiments —Y³— of formula (XII) is —N(R¹³).

In certain embodiments —Y¹— of formula (XII) is —N(R¹¹)—, ═Y of formula (XII) is ═O and —Y³— is —O—.

In certain embodiments —Y¹— of formula (XII) is —N(R¹¹)—, ═Y² of formula (XII) is ═O, —Y³— of formula (XII) is —O— and -Nu of formula (XII) is —N(CH₃)₂.

In certain embodiments -E- of formula (XII) is selected from the group consisting of C₁₋₆ alkyl, C₂₋₆ alkenyl, C₂₋₆ alkynyl and -Q-. In certain embodiments -E- of formula (XII) is C₁₋₆ alkyl. In certain embodiments -E- of formula (XII) is C₂₋₆ alkenyl. In certain embodiments -E- of formula (XII) is C₂₋₆ alkynyl. In certain embodiments -E- of formula (XII) is -Q-.

In certain embodiments Q of formula (XII) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments Q of formula (XII) is phenyl. In certain embodiments Q of formula (XII) is naphthyl. In certain embodiments Q of formula (XII) is indenyl. In certain embodiments Q of formula (XII) is indanyl. In certain embodiments Q of formula (XII) is tetralinyl. In certain embodiments Q of formula (XII) is C₃₋₁₀ cycloalkyl. In certain embodiments Q of formula (XII) is 3- to 10-membered heterocyclyl. In certain embodiments Q of formula (XII) is 8- to 11-membered heterobicyclyl. In certain embodiments Q of formula (XII) is substituted with one or more —R¹⁴. In certain embodiments Q of formula (XII) is not substituted with —R¹⁴.

In certain embodiments —R⁵, —R⁶, each —R⁷, —R⁸, —R⁹, —R¹⁰, —R^{10a}, —R¹¹, —R¹² and —R¹³ of formula (XII) are independently selected from the group consisting of C₁₋₂₀ alkyl, C₂₋₂₀ alkenyl, C₂₋₂₀ alkynyl and -Q.

In certain embodiments —R⁵ of formula (XII) is C₁₋₂₀ alkyl. In certain embodiments —R⁵ of formula (XII) is C₂₋₂₀ alkenyl. In certain embodiments —R⁵ of formula (XII) is C₂₋₂₀ alkynyl. In certain embodiments —R⁵ of formula (XII) is -Q.

In certain embodiments —R⁶ of formula (XII) is C₁₋₂₀ alkyl. In certain embodiments —R⁶ of formula (XII) is C₂₋₂₀ alkenyl. In certain embodiments —R⁶ of formula (XII) is C₂₋₂₀ alkynyl. In certain embodiments —R⁶ is -Q.

In certain embodiments each of —R⁷ of formula (XII) is independently selected from the group consisting of C₁₋₂₀ alkyl, C₂₋₂₀ alkenyl, C₂₋₂₀ alkynyl and -Q. In certain embodiments each of —R⁷ of formula (XII) is C₁₋₂₀ alkyl. In certain embodiments each of —R⁷ of formula (XII) is C₂₋₂₀ alkenyl. In certain embodiments each of —R⁷ of formula (XII) is C₂₋₂₀ alkynyl. In certain embodiments each of —R⁷ of formula (XII) is -Q.

In certain embodiments —R⁸ of formula (XII) is C₁₋₂₀ alkyl. In certain embodiments —R⁸ of formula (XII) is C₂₋₂₀ alkenyl. In certain embodiments —R⁸ of formula (XII) is C₂₋₂₀ alkynyl. In certain embodiments —R⁸ of formula (XII) is -Q.

In certain embodiments —R⁹ of formula (XII) is C₁₋₂₀ alkyl. In certain embodiments —R⁹ of formula (XII) is C₂₋₂₀ alkenyl. In certain embodiments —R⁹ of formula (XII) is C₂₋₂₀ alkynyl. In certain embodiments —R⁹ of formula (XII) is -Q.

In certain embodiments —R¹⁰ of formula (XII) is C₁₋₂₀ alkyl. In certain embodiments —R¹⁰ of formula (XII) is C₂₋₂₀ alkenyl. In certain embodiments —R¹⁰ of formula (XII) is C₂₋₂₀ alkynyl. In certain embodiments —R¹⁰ of formula (XII) is -Q.

In certain embodiments —R^{10a} of formula (XII) is C₁₋₂₀ alkyl. In certain embodiments —R^{10a} of formula (XII) is C₂₋₂₀ alkenyl. In certain embodiments —R^{10a} of formula (XII) is C₂₋₂₀ alkynyl. In certain embodiments —R^{10a} of formula (XII) is -Q.

In certain embodiments —R¹¹ of formula (XII) is C₁₋₂₀ alkyl. In certain embodiments —R¹¹ of formula (XII) is C₂₋₂₀ alkenyl. In certain embodiments —R¹¹ of formula (XII) is C₂₋₂₀ alkynyl. In certain embodiments —R¹¹ of formula (XII) is -Q.

In certain embodiments —R¹² of formula (XII) is C₁₋₂₀ alkyl. In certain embodiments —R¹² of formula (XII) is C₂₋₂₀ alkenyl. In certain embodiments —R¹² of formula (XII) is C₂₋₂₀ alkynyl. In certain embodiments —R¹² of formula (XII) is -Q.

In certain embodiments —R¹³ of formula (XII) is C₁₋₂₀ alkyl. In certain embodiments —R¹³ of formula (XII) is C₂₋₂₀ alkenyl. In certain embodiments —R¹³ of formula (XII) is C₂₋₂₀ alkynyl. In certain embodiments —R¹² of formula (XII) is -Q.

In certain embodiments —R¹⁴, —R¹⁵ and —R^{15a} of formula (XII) are selected from the group consisting of —H and C₁₋₆ alkyl.

In certain embodiments —R¹⁴ of formula (XII) is —H. In certain embodiments —R¹⁴ of formula (XII) is C₁₋₆ alkyl.

In certain embodiments —R¹⁵ of formula (XII) is —H. In certain embodiments —R¹⁵ of formula (XII) is C₁₋₆ alkyl.

In certain embodiments —R^{15a} of formula (XII) is —H. In certain embodiments —R^{15a} of formula (XII) is C₁₋₆ alkyl.

In certain embodiments —Y of formula (XII) is

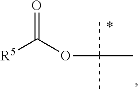

wherein —R⁵ is as defined above and the dashed line marked with an asterisk indicates the attachment to -A-.

In certain embodiments —Y of formula (XII) is

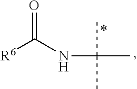

wherein —R⁶ is as defined above and the dashed line marked with an asterisk indicates the attachment to -A-.

In certain embodiments —R⁶ of formula (XII) is of formula (XIIa):

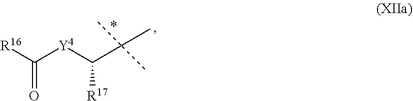

wherein —Y⁴— is selected from the group consisting of C₃₋₁₀ cycloalkyl, 3- to 10-membered heterocyclyl and 8- to 11-membered heterobicyclyl, which are optionally substituted with one or more —R¹⁸ which are the same or different;
—R¹⁶ and —R¹⁷ are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R¹⁸ which are the same or different; and wherein C₁₋₁₀ alkyl, C₂₋₁₀ alkenyl and C₂₋₁₀ alkynyl are optionally interrupted by one or more groups selected from the group consisting of -A′-, —C(O)O—, —O—, —C(O)—, —C(O)N(R¹⁹)—, —S(O)₂N(R¹⁹), —S(O)N(R¹⁹)—, —S(O)₂—, —S(O)—, —N(R¹⁹)S(O)₂N(R^{19a})—, —S—, —N(R¹⁹)—, —OC(OR¹⁹)R^{19a}—, —N(R¹⁹)C(O)N(R^{19a})—, —OC(O)N(R¹⁹)— and —N(R¹⁹)C(NH)N(R^{19a})—;
   each A′ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each A′ is independently optionally substituted with one or more —R¹⁸ which are the same or different;
   wherein —R¹⁸, —R¹⁹ and —R^{19a} are independently selected from the group consisting of —H and C₁₋₆ alkyl; wherein C₁₋₆ alkyl is optionally substituted with one or more halogen, which are the same or different; and
wherein the dashed line marked with an asterisk indicates the attachment to the rest of —Y.

In certain embodiments —Y⁴— of formula (XIIa) is selected from the group consisting of C₃₋₁₀ cycloalkyl, 3- to 10-membered heterocyclyl and 8- to 11-membered heterobicyclyl. In certain embodiments —Y⁴— of formula (XIIa) is C₃₋₁₀ cycloalkyl. In certain embodiments —Y⁴— of formula (XIIa) is 3- to 10-membered heterocyclyl. In certain embodiments —Y⁴— of formula (XIIa) is 8- to 11-membered heterobicyclyl. In certain embodiments —Y⁴— of formula (XIIa) is substituted with one or more —R¹⁸ which are the same or different. In certain embodiments —Y⁴— of formula (XIIa) is not substituted with —R¹⁸.

In certain embodiments —R¹⁶ and —R¹⁷ of formula (XIIa) are selected from the group consisting of C₁₋₁₀ alkyl, C₂₋₁₀ alkenyl and C₂₋₁₀ alkynyl. In certain embodiments —R¹⁶ of formula (XIIa) is C₁₋₁₀ alkyl. In certain embodiments —R¹⁶ of formula (XIIa) is C₂₋₁₀ alkenyl. In certain embodiments —R¹⁶ of formula (XIIa) is C₂₋₁₀ alkynyl. In certain embodiments —R¹⁷ of formula (XIIa) is C₁₋₁₀ alkyl. In certain embodiments —R¹⁷ of formula (XIIa) is C₂₋₁₀ alkenyl. In certain embodiments —R¹⁷ of formula (XIIa) is C₂₋₁₀ alkynyl.

In certain embodiments A′ of formula (XIIa) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments A′ of formula (XIIa) is phenyl. In certain embodiments A′ of formula (XIIa) is naphthyl. In certain embodiments A′ of formula (XIIa) is indenyl. In certain embodiments A′ of formula (XIIa) is indanyl. In certain embodiments A′ of formula (XIIa) is tetralinyl. In certain embodiments A′ of formula (XIIa) is C₃₋₁₀ cycloalkyl. In certain embodiments A′ of formula (XIIa) is 3- to 10-membered heterocyclyl. In certain embodiments A′ of formula (XIIa) is 8- to 11-membered heterobicyclyl.

In certain embodiments A′ of formula (XIIa) is substituted with one or more —R¹⁸, which are the same or different. In certain embodiments A′ of formula (XIIa) is not substituted with —R¹⁸.

In certain embodiments —R¹⁸, —R¹⁹ and —R^{19a} of formula (XIIa) are selected from the group consisting of —H and C₁₋₆ alkyl.

In certain embodiments —R¹⁸ of formula (XIIa) is —H. In certain embodiments —R¹⁸ of formula (XIIa) is C₁₋₆ alkyl. In certain embodiments —R¹⁹ of formula (XIIa) is —H. In certain embodiments —R¹⁹ of formula (XIIa) is C₁₋₆ alkyl. In certain embodiments —R^{19a} of formula (XIIa) is —H. In certain embodiments —R^{19a} of formula (XIIa) is C₁₋₆ alkyl.

In certain embodiments —R⁶ of formula (XII) is of formula (XIIb):

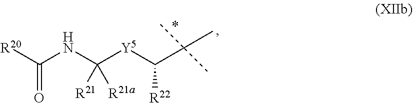

wherein —Y⁵— is selected from the group consisting of -Q′-, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²³, which are the same or different; and wherein C₁₋₁₀ alkyl, C₂₋₁₀ alkenyl and C₂₋₁₀ alkynyl are optionally interrupted by one or more groups selected from the group consisting of -Q′-, —C(O)O—, —O—, —C(O)—, —C(O)N(R²⁴)—, —S(O)₂N(R²⁴), —S(O)N(R²⁴)—, —S(O)₂—, —S(O)—, —N(R²⁴)S(O)₂N(R^{24a})—, —S—, —N(R²⁴)—, —OC(OR²⁴)R^{24a}—, —N(R²⁴)C(O)N(R^{24a})—, —OC(O)N(R²⁴)— and —N(R²⁴)C(NH)N(R^{24a})—; —R²⁰, —R²¹, —R^{21a} and —R²² are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²³ which are the same or different; and wherein C₁₋₁₀ alkyl, C₂₋₁₀ alkenyl and C₂₋₁₀ alkynyl are optionally interrupted by one or more groups selected from the group consisting of -Q-, —C(O)O—, —O—, —C(O)—,
—C(O)N(R²⁴)—, —S(O)₂N(R²⁴), —S(O)N(R²⁴)—, —S(O)₂—, —S(O)—, —N(R²⁴)S(O)₂N(R^{24a})—, —S—, —N(R²⁴)—, —OC(OR²⁴)R^{24a}—, —N(R²⁴)C(O)N(R^{24a})—, —OC(O)N(R²⁴)— and —N(R²⁴)C(NH)N(R^{24a})—;
   each Q′ is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q′ is independently optionally substituted with one or more —R²³, which are the same or different;
   wherein —R²³, —R²⁴ and —R^{24a} are independently selected from the group consisting of —H and C₁₋₆ alkyl; wherein C₁₋₆ alkyl is optionally substituted with one or more halogen, which are the same or different;
optionally, the pair —R²¹/—R^{21a} is joined together with the atoms to which is attached to form a C₃₋₁₀ cycloalkyl, 3- to 10-membered heterocyclyl or an 8- to 11-membered heterobicyclyl; and
wherein the dashed line marked with an asterisk indicates the attachment to the rest of —Y.

In certain embodiments —Y⁵— of formula (XIIb) is selected from the group consisting of -Q-, C₁₋₁₀ alkyl, C₂₋₁₀ alkenyl and C₂₋₁₀ alkynyl. In certain embodiments —Y⁵— of formula (XIIb) is -Q′-. In certain embodiments —Y⁵— of formula (XIIb) is C₁₋₁₀ alkyl. In certain embodiments —Y⁵— of formula (XIIb) is C₂₋₁₀ alkenyl. In certain embodiments —Y⁵— of formula (XIIb) is C₂₋₁₀ alkynyl.

In certain embodiments Q′ of formula (XIIb) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments Q′ of formula (XIIb) is phenyl. In certain embodiments Q′ of formula (XIIb) is naphthyl. In certain embodiments Q′ of formula (XIIb) is indenyl. In certain embodiments Q′ of formula (XIIb) is indanyl. In certain embodiments Q′ of formula (XIIb) is C₃₋₁₀ cycloalkyl. In certain embodiments Q′ of formula (XIIb) is 3- to 10-membered heterocyclyl. In certain embodiments Q′ of formula (XIIb) is 8- to 11-membered heterobicyclyl. In certain embodiments Q′ of formula (XIIb) is substituted with one or more —R²³ which are the same or different. In certain embodiments Q′ of formula (XIIb) is not substituted with —R²³.

In certain embodiments —R²⁰, —R²¹, —R^{21a} and —R²² of formula (XIIb) are selected from the group consisting of —H, C₁₋₁₀ alkyl, C₂₋₁₀ alkenyl and C₂₋₁₀ alkynyl. In certain embodiments —R of formula (XIIb) is —H. In certain embodiments —R²⁰ of formula (XIIb) is C₁₋₁₀ alkyl. In certain embodiments —R²⁰ of formula (XIIb) is C₂₋₁₀ alkenyl. In certain embodiments —R²⁰ of formula (XIIb) is C₂₋₁₀ alkynyl. In certain embodiments —R²¹ of formula (XIIb) is —H. In certain embodiments —R²¹ of formula (XIIb) is C₁₋₁₀ alkyl. In certain embodiments —R²¹ of formula (XIIb) is C₂₋₁₀ alkenyl. In certain embodiments —R^{21a} of formula (XIIb) is C₂₋₁₀ alkynyl. In certain embodiments —R^{21a} of formula (XIIb) is —H. In certain embodiments —R^{21a} of formula (XIIb) is C₁₋₁₀ alkyl. In certain embodiments —R^{21a} of formula (XIIb) is C₂₋₁₀ alkenyl. In certain embodiments —R^{21a} of formula (XIIb) is C₂₋₁₀ alkynyl. In certain embodiments —R²² of formula (XIIb) is —H. In certain embodiments —R²² of formula (XIIb) is C₁₋₁₀ alkyl. In certain embodiments —R²² of formula (XIIb) is C₂₋₁₀ alkenyl. In certain embodiments —R²² of formula (XIIb) is C₂₋₁₀ alkynyl.

In certain embodiments —R²³, —R²⁴ and —R^{24a} of formula (XIIb) are selected from the group consisting of —H and C₁₋₆ alkyl. In certain embodiments —R²³ of formula (XIIb) is —H. In certain embodiments —R²³ of formula (XIIb) is C₁₋₆ alkyl. In certain embodiments —R²⁴ of formula (XIIb) is —H. In certain embodiments —R²⁴ of formula (XIIb) is C₁₋₆ alkyl. In certain embodiments —R^{24a} of formula (XIIb) is —H. In certain embodiments —R^{24a} of formula (XIIb) is C₁₋₆ alkyl.

In certain embodiments the pair —R²¹/—R^{21a} of formula (XIIb) is joined together with the atoms to which is attached to form a C₃₋₁₀ cycloalkyl.

In certain embodiments —R⁶ of formula (XIIb) is of formula (XIIc):

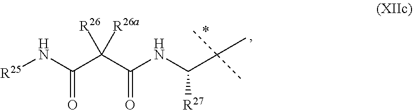

wherein
—R²⁵, —R²⁶, —R^{26a} and —R²⁷ are independently selected from the group consisting of —H, C₁₋₁₀ alkyl, C₂₋₁₀ alkenyl and C₂₋₁₀ alkynyl; wherein C₁₋₁₀ alkyl, C₂₋₁₀ alkenyl and C₂₋₁₀ alkynyl are optionally substituted with one or more —R²⁸ which are the same or different; and wherein C₁₋₁₀ alkyl, C₂₋₁₀ alkenyl and C₂₋₁₀ alkynyl are optionally interrupted by one or more groups selected from the group consisting of -Q*-, —C(O)O—, —O—, —C(O)—, —C(O)N(R²⁹)—, —S(O)₂N(R²⁹), —S(O)N(R²⁹)—, —S(O)₂—, —S(O)—, —N(R²⁹)S(O)₂N(R^{29a})—, —S—, —N(R²⁹)—, —OC(OR²⁹)R^{29a}—, —N(R²⁹)C(O)N(R^{29a})—, —OC(O)N(R²⁹)— and —N(R²⁹)C(NH)N(R^{29a})—;
   each Q* is independently selected from the group consisting of phenyl, naphthyl, indenyl, indanyl, tetralinyl, C₃₋₁₀ cycloalkyl, 3- to 10-membered heterocyclyl and 8- to 11-membered heterobicyclyl, wherein each Q* is independently optionally substituted with one or more —R²⁸, which are the same or different;
   wherein —R²⁸, —R²⁹ and —R^{29a} are independently selected from the group consisting of —H and C₁₋₆ alkyl; wherein C₁₋₆ alkyl is optionally substituted with one or more halogen, which are the same or different;
optionally, the pair —R²⁶/—R^{26a} is joined together with the atoms to which is attached to form a C₃₋₁₀ cycloalkyl, 3- to 10-membered heterocyclyl or an 8- to 11-membered heterobicyclyl; and
wherein the dashed line marked with an asterisk indicates the attachment to the rest of —Y.

In certain embodiments —R²⁵, —R²⁶, —R^{26a} and —R²⁷ of formula (XIIc) are selected from the group consisting of —H, C₁₋₁₀ alkyl, C₂₋₁₀ alkenyl and C₂₋₁₀ alkynyl. In certain embodiments —R of formula (XIIc) is —H. In certain embodiments —R²⁵ of formula (XIIc) is C₁₋₁₀ alkyl. In certain embodiments —R²⁵ of formula (XIIc) is C₂₋₁₀ alkenyl. In certain embodiments —R²⁵ of formula (XIIc) is C₂₋₁₀ alkynyl. In certain embodiments —R²⁶ of formula (XIIc) is —H. In certain embodiments —R²⁶ of formula (XIIc) is C₁₋₁₀ alkyl. In certain embodiments —R²⁶ of formula (XIIc) is C₂₋₁₀ alkenyl. In certain embodiments —R²⁶ of formula (XIIc) is C₂₋₁₀ alkynyl. In certain embodiments —R^{26a} of formula (XIIc) is —H. In certain embodiments —R^{26a} of formula (XIIc) is C₁₋₁₀ alkyl. In certain embodiments —R^{26a} of formula (XIIc) is C₂₋₁₀ alkenyl. In certain embodiments —R^{26a} of formula (XIIc) is C₂₋₁₀ alkynyl. In certain embodiments —R²⁷ of formula (XIIc) is —H. In certain embodiments —R²⁷ of formula (XIIc) is C₁₋₁₀ alkyl. In certain embodiments —R²⁷ of formula (XIIc) is C₂₋₁₀ alkenyl. In certain embodiments —R²⁷ of formula (XIIc) is C₂₋₁₀ alkynyl.

In certain embodiments Q* of formula (XIIc) is selected from the group consisting of phenyl, naphthyl, indenyl, indanyl, tetralinyl, C₃₋₁₀ cycloalkyl, 3- to 10-membered heterocyclyl and 8- to 11-membered heterobicyclyl. In certain embodiments Q* of formula (XIIc) is phenyl. In certain embodiments Q* of formula (XIIc) is naphthyl. In certain embodiments Q* of formula (XIIc) is indenyl. In certain embodiments Q* of formula (XIIc) is indanyl. In certain embodiments Q* of formula (XIIc) is tetralinyl. In certain embodiments Q* of formula (XIIc) is C₃₋₁₀ cycloalkyl. In certain embodiments Q* of formula (XIIc) is 3- to 10-membered heterocyclyl. In certain embodiments Q* of formula (XIIc) is 8- to 11-membered heterobicyclyl. In certain embodiments Q* of formula (XIIc) is substituted with one or more —R²⁸, which are the same or different. In certain embodiments Q* of formula (XIIc) is not substituted with —R²⁸.

In certain embodiments —R²⁸, —R²⁹ and —R^{29a} of formula (XIIc) are selected from the group consisting of —H and C₁₋₆ alkyl. In certain embodiments —R²⁸ of formula (XIIc) is —H. In certain embodiments —R²⁸ of formula (XIIc) is C₁₋₆ alkyl. In certain embodiments —R²⁹ of formula (XIIc) is —H. In certain embodiments —R²⁹ of formula (XIIc) is C₁₋₆ alkyl. In certain embodiments —R^{29a} of formula (XIIc) is —H. In certain embodiments —R^{29a} of formula (XIIc) is C₁₋₆ alkyl.

In certain embodiments the pair —R²⁶/—R^{26a} of formula (XIIc) is joined together with the atoms to which is attached to form a C₃₋₁₀ cycloalkyl. In certain embodiments the pair —R²⁶/—R^{26a} of formula (XIIc) is joined together with the atoms to which is attached to form a cyclobutyl.

In certain embodiments —Y of formula (XII) is

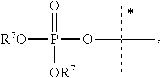

wherein each —R⁷ is as defined above and the dashed line marked with an asterisk indicates the attachment to -A-. It is understood that in this instance the release of the drug D may be triggered by an enzyme, such as phosphatase.

In certain embodiments —Y of formula (XII) is

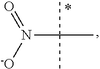

wherein the dashed line marked with an asterisk indicates the attachment to -A-.

In certain embodiments —Y of formula (XII) is

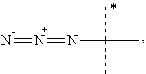

wherein the dashed line marked with an asterisk indicates the attachment to -A-.

In certain embodiments —Y of formula (XII) is

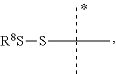

wherein —R⁸ is as defined above and the dashed line marked with an asterisk indicates the attachment to -A-.

In certain embodiments —Y of formula (XII) is

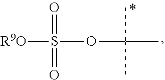

wherein —R⁹ is as defined above and the dashed line marked with an asterisk indicates the attachment to -A-. It is understood that in this instance the release of the drug D may be triggered by an enzyme, such as sulfatase.

In certain embodiments —Y of formula (XII) is

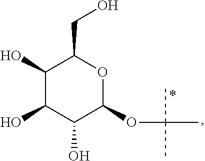

wherein the dashed line marked with an asterisk indicates the attachment to -A-. It is understood that in this instance the release of the drug D may be triggered by an enzyme, such as α-galactosidase.

In certain embodiments —Y of formula (XII) is

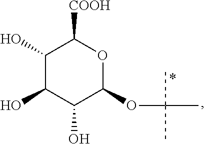

wherein the dashed line marked with an asterisk indicates the attachment to -A-. It is understood that in this instance the release of the drug D may be triggered by an enzyme, such as β-glucuronidase.

In certain embodiments —Y of formula (XII) is

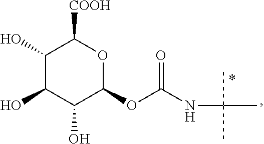

wherein the dashed line marked with an asterisk indicates the attachment to -A-. It is understood that in this instance the release of the drug D may be triggered by an enzyme, such as β-glucuronidase.

In certain embodiments —Y of formula (XII) is a peptidyl moiety.

It is understood that if —Y of formula (XII) is a peptidyl moiety, then the release of the drug D may be triggered by an enzyme, such as protease. In certain embodiments the protease is selected from the group consisting of cathepsin B and cathepsin K. In certain embodiments the protease is cathepsin B. In certain embodiments the protease is cathepsin K.

In certain embodiments —Y of formula (XII) is a peptidyl moiety, such as a dipeptidyl, tripeptidyl, tetrapeptidyl, pentapeptidyl or hexapeptidyl moiety. In certain embodiments —Y of formula (XII) is a dipeptidyl moiety. In certain embodiments —Y of formula (XII) is a tripeptidyl moiety. In certain embodiments —Y of formula (XII) is a tetrapeptidyl moiety. In certain embodiments —Y of formula (XII) is a pentapeptidyl moiety. In certain embodiments —Y of formula (XII) is a hexapeptidyl moiety.

In certain embodiments —Y of formula (XII) is a peptidyl moiety selected from the group consisting of:

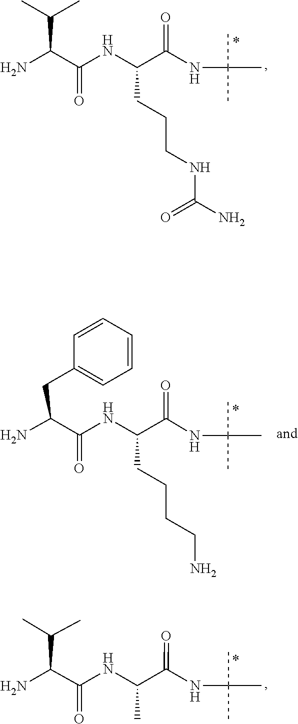

wherein the dashed line marked with an asterisk indicates the attachment to -A-.

In certain embodiments —Y of formula (XII) is

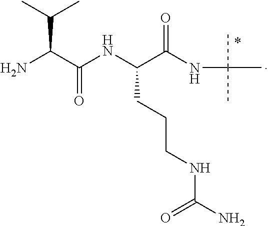

In certain embodiments —Y of formula (XII) is

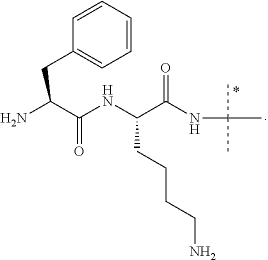

In certain embodiments —Y of formula (XII) is

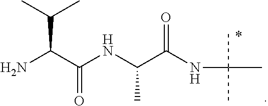

In certain embodiments one hydrogen given by —R^{1a} of formula (XII) is replaced by -L²- and -L¹- is of formula (XII′):

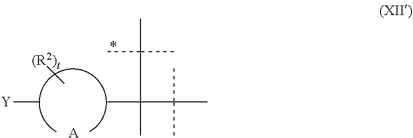

wherein
the unmarked dashed line indicates the attachment to the N⁺ of -D⁺, the dashed line marked with an asterisk indicates the attachment to -L²-; and
—R¹, —Ar—, —Y, R² and t are defined as in formula (XII).

In certain embodiments one hydrogen given by —R² of formula (XII) is replaced by -L²- and -L¹- is of formula (XII″):

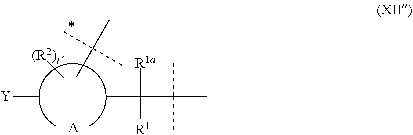

wherein
the unmarked dashed line indicates the attachment to the N⁺ of -D⁺, the dashed line marked with an asterisk indicates the attachment to -L²-;
—R¹, —Ar—, —Y and R² are defined as in formula (XII); and
t′ is selected from the group consisting of 0, 1, 2, 3, 4 and 5.

In certain embodiments t′ of formula (XII″) is 0. In certain embodiments t′ of formula (XII″) is 1. In certain embodiments t′ of formula (XII″) is 2. In certain embodiments t′ of formula (XII″) is 3. In certain embodiments t′ of formula (XII″) is 4. In certain embodiments t′ of formula (XII″) is 5.

It is understood that the phrase “-L¹- is substituted with —X^{0D}-L²-” means that -L²- is attached to -L¹- via —X^{0D}—, which is either absent or a linkage, and that the moiety —X^{0D}-L²- is not attached to -L¹- via -L²-.

-L²- is a chemical bond or a spacer moiety. In certain embodiments -L²- does not comprise a reversible linkage, i.e. all linkages in -L²- are stable linkages. -L¹- is connected to -L²- via a stable linkage. -L²- is connected to —Z via a stable linkage.

In certain embodiments -L²- is a chemical bond.

In certain embodiments -L²- is a spacer moiety.

In certain embodiments -L²- is a spacer moiety selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₅₀ alkyl, C₂₋₅₀ alkenyl, and C₂₋₅₀ alkynyl; wherein -T, C₁₋₅₀ alkyl, C₂₋₅₀ alkenyl, and C₂₋₅₀ alkynyl are optionally substituted with one or more —R^{y2}, which are the same or different, and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

each —R^{y2} is independently selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5})C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently selected from the group consisting of —H, and C₁₋₆ alkyl, wherein C₁₋₆ alkyl is optionally substituted with one or more halogen, which are the same or different.

In certain embodiments -L²- is a spacer moiety selected from -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₂₀ alkyl, C₂₋₂₀ alkenyl, and C₂₋₂₀ alkynyl are optionally substituted with one or more —R^{y2}, which are the same or different and wherein C₁₋₂₀ alkyl, C₂₋₂₀ alkenyl, and C₂₋₂₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently of each other selected from the group consisting of —H, -T, C₁₋₁₀ alkyl, C₂₋₁₀ alkenyl, and C₂₋₁₀ alkynyl; wherein -T, C₁₋₁₀ alkyl, C₂₋₁₀ alkenyl, and C₂₋₁₀ alkynyl are optionally substituted with one or more —R^{y2}, which are the same or different, and wherein C₁₋₁₀ alkyl, C₂₋₁₀ alkenyl, and C₂₋₁₀ alkynyl are optionally interrupted by one or more groups selected from the group consisting of -T-, —C(O)O—, —O—, —C(O)—, —C(O)N(R^{y4})—, —S(O)₂N(R^{y4})—, —S(O)N(R^{y4})—, —S(O)₂—, —S(O)—, —N(R^{y4})S(O)₂N(R^{y4a})—, —S—, —N(R^{y4})—, —OC(OR^{y4})(R^{y4a})—, —N(R^{y4})C(O)N(R^{y4a})—, and —OC(O)N(R^{y4})—;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl; wherein each T is independently optionally substituted with one or more —R^{y2}, which are the same or different;

—R^{y2} is selected from the group consisting of halogen, —CN, oxo (═O), —COOR^{y5}, —OR^{y5}, —C(O)R^{y5}, —C(O)N(R^{y5}R^{y5a}), —S(O)₂N(R^{y5}R^{y5a}), —S(O)N(R^{y5}R^{y5a}), —S(O)₂R^{y5}, —S(O)R^{y5}, —N(R^{y5})S(O)₂N(R^{y5a}R^{y5b}), —SR^{y5}, —N(R^{y5}R^{y5a}), —NO₂, —OC(O)R^{y5}, —N(R^{y5}) C(O)R^{y5a}, —N(R^{y5})S(O)₂R^{y5a}, —N(R^{y5})S(O)R^{y5a}, —N(R^{y5})C(O)OR^{y5a}, —N(R^{y5})C(O)N(R^{y5a}R^{y5b}), —OC(O)N(R^{y5}R^{y5a}), and C₁₋₆ alkyl; wherein C₁₋₆ alkyl is optionally substituted with one or more halogen, which are the same or different; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is a spacer moiety selected from the group consisting of -T-, —C(O)O—, —O—, —C(O)—, —C(O)N(R^{y1})—, —S(O)₂N(R^{y1})—, —S(O)N(R^{y1})—, —S(O)₂—, —S(O)—, —N(R^{y1})S(O)₂N(R^{y1a})—, —S—, —N(R^{y1})—, —OC(OR^{y1})(R^{y1a})—, —N(R^{y1})C(O)N(R^{y1a})—, —OC(O)N(R^{y1})—, C₁₋₅₀ alkyl, C₂₋₅₀ alkenyl, and C₂₋₅₀ alkynyl; wherein -T-, C₁₋₅₀ alkyl, C₂₋₅₀ alkenyl, and C₂₋₅₀ alkynyl are optionally substituted with one or more —R^{y2}, which are the same or different and wherein C₁₋₅₀ alkyl, C₂₋₅₀ alkenyl, and C₂₋₅₀ alkynyl are optionally interrupted by one or more groups selected from the group consisting of -T-, —C(O)O—, —O—, —C(O)—, —C(O)N(R^{y3})—, —S(O)₂N(R^{y3})—, —S(O)N(R^{y3})—, —S(O)₂—, —S(O)—, —N(R^{y3})S(O)₂N(R^{y3a})—, —S—, —N(R^{y3})—, —OC(OR^{y3})(R^{y3a})—, —N(R^{y3})C(O)N(R^{y3a})—, and —OC(O)N(R^{y3})—;

—R^{y1} and —R^{y1a} are independently selected from the group consisting of —H, -T, C₁₋₁₀ alkyl, C₂₋₁₀ alkenyl, and C₂₋₁₀ alkynyl;

each T is independently selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl;

each —R^{y2} is independently selected from the group consisting of halogen, and C₁₋₆ alkyl; and

each —R^{y3}, —R^{y3a}, —R^{y4}, —R^{y4a}, —R^{y5}, —R^{y5a} and —R^{y5b} is independently of each other selected from the group consisting of —H, and C₁₋₆ alkyl; wherein C₁₋₆ alkyl is optionally substituted with one or more halogen, which are the same or different.

In certain embodiments -L²- is a C₁₋₂₀ alkyl chain, which is optionally interrupted by one or more groups independently selected from —O—, -T- and —C(O)N(R^{y1})—; and which C₁₋₂₀ alkyl chain is optionally substituted with one or more groups independently selected from —OH, -T and —C(O)N(R^{y6}R^{y6a}); wherein —R^{y1}, —R^{y6}, —R^{y6a} are independently selected from the group consisting of H and C₁₋₄ alkyl and wherein T is selected from the group consisting of phenyl, naphthyl, indenyl, indanyl, tetralinyl, C₃₋₁₀ cycloalkyl, 3- to 10-membered heterocyclyl, 8- to 11-membered heterobicyclyl, 8- to 30-membered carbopolycyclyl, and 8- to 30-membered heteropolycyclyl.

In certain embodiments -L²- has a molecular weight in the range of from 14 g/mol to 750 g/mol.

In certain embodiments -L²- comprises a moiety selected from

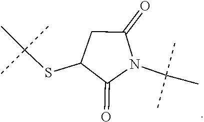

In certain embodiments -L²- has a chain lengths of 1 to 20 atoms.

As used herein the term “chain length” with regard to the moiety -L²- refers to the number of atoms of -L²- present in the shortest connection between -L¹- and —Z.

In certain embodiments -L²- is of formula (i)

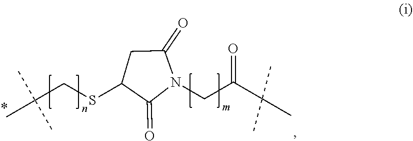

wherein
the dashed line marked with the asterisk indicates attachment to -L¹-;
the unmarked dashed line indicates attachment to —Z;
n is selected from the group consisting of 0, 1, 2, 3, 4, 5, 6, 7, 8, 9, 10, 11, 12, 13, 14, 15, 16, 17 and 18;
m is selected from the group consisting of 0, 1, 2, 3, 4, 5, 6, 7, 8, 9, 10, 11, 12, 13, 14, 15, 16, 17 and 18; and
wherein the moiety of formula (i) is optionally further substituted.

In certain embodiments n of formula (i) is selected from the group consisting of 3, 4, 5, 6, 7, 8, and 9. In certain embodiments n of formula (i) is 3, 4, 5, 6, or 7. In certain embodiments n of formula (i) is 3. In certain embodiments n of formula (i) is 4. In certain embodiments n of formula (i) is 5. In certain embodiments n of formula (i) is 6.

In certain embodiments m of formula (i) is selected from the group consisting of 1, 2, 3, 4, 5, 6 or 7. In certain embodiments m of formula (i) is 1, 2, 3, 4 or 5. In certain embodiments m of formula (i) is 1. In certain embodiments m of formula (i) is 2. In certain embodiments m of formula (i) is 3. In certain embodiments m of formula (i) is 4.

In certain embodiments the moiety -L¹-L²- is selected from the group consisting of

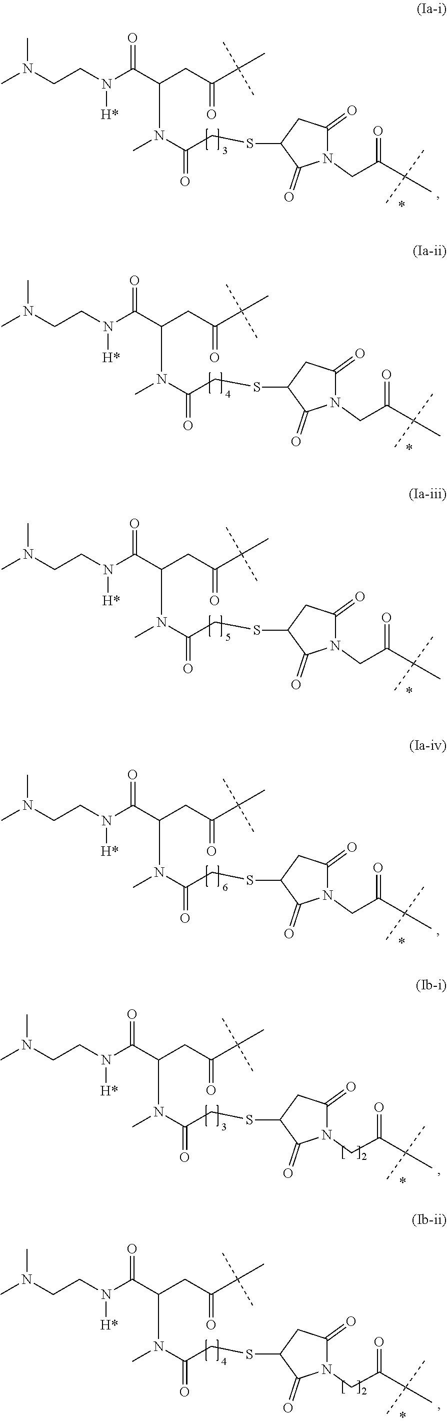

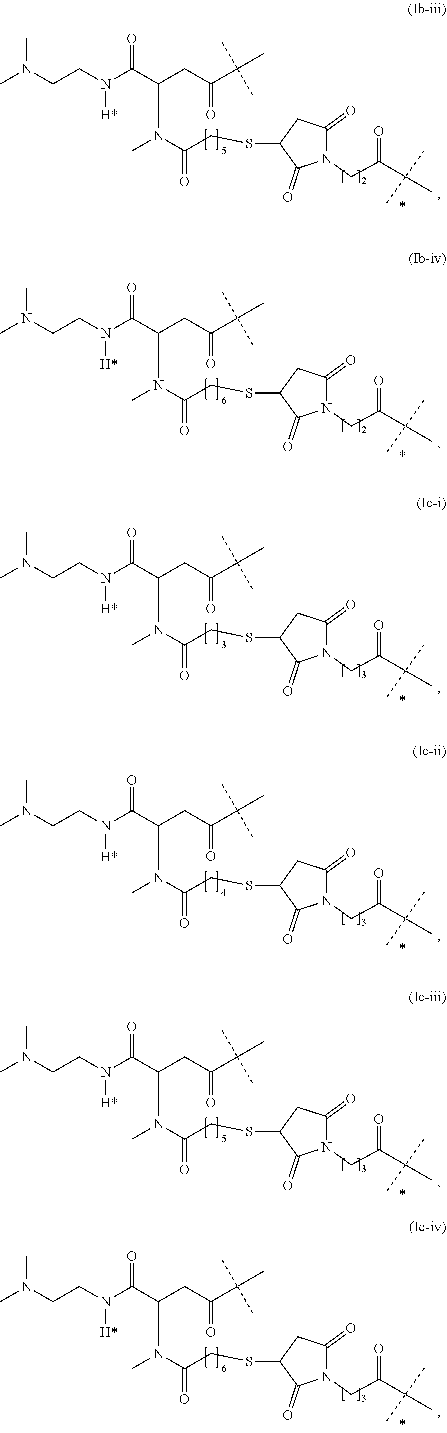

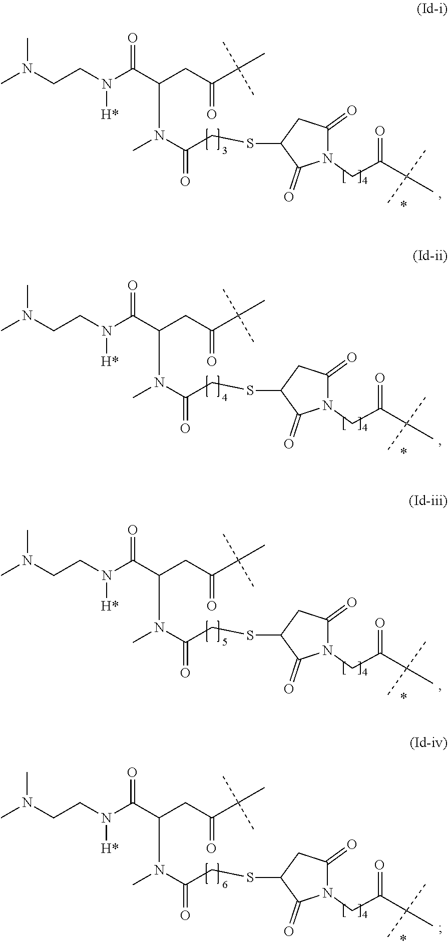

wherein
the unmarked dashed line indicates the attachment to a nitrogen of -D by forming an amide bond; and
the dashed line marked with the asterisk indicates attachment to —Z.

In certain embodiments the moiety -L¹-L²- is of formula (IIa-i). In certain embodiments the moiety -L¹-L²- is of formula (IIa-ii). In certain embodiments the moiety -L¹-L²- is of formula (IIa-iii). In certain embodiments the moiety -L¹-L²- is of formula (IIa-iv). In certain embodiments the moiety -L¹-L²f- is of formula (IIb-i). In certain embodiments the moiety -L¹-L²- is of formula (IIb-ii). In certain embodiments the moiety -L¹-L²- is of formula (IIb-iii). In certain embodiments the moiety -L¹-L²- is of formula (IIb-iv). In certain embodiments the moiety -L¹-L²- is of formula (IIc-i). In certain embodiments the moiety -L¹-L²- is of formula (IIc-ii). In certain embodiments the moiety -L¹-L²- is of formula (IIc-iii). In certain embodiments the moiety -L¹-L²- is of formula (IIc-iv). In certain embodiments the moiety -L¹-L²- is of formula (IId-i). In certain embodiments the moiety -L¹-L²- is of formula (IId-ii). In certain embodiments the moiety -L¹-L²- is of formula (IId-iii). In certain embodiments the moiety -L¹-L²- is of formula (IId-iv).

The conjugates of the present invention release one or more type of drug, such as an antibiotic or other type of drug, over an extended period of time, i.e. they are sustained-release conjugates. In certain embodiments the release occurs with a release half-life ranging between 1 day and 1 month. In certain embodiments the release occurs with a release half-life ranging between 1 day and 20 days. In certain embodiments the release occurs with a release half-life between 1 day and 15 days. In certain embodiments the release half-life may also range from 2 to 20 days or from 4 to 15 days.

In another aspect the present invention relates to a pharmaceutical composition comprising a conjugate of the present invention and at least one excipient. It is understood that more than one type of conjugate of the present invention may be present in such pharmaceutical composition.

Such pharmaceutical composition may have a pH ranging from pH 3 to pH 8, such as ranging from pH 4 to pH 6 or ranging from pH 4 to pH 5. In certain embodiments the pH of such pharmaceutical composition is about 4. In certain embodiments the pH of such pharmaceutical composition is about 4.5. In certain embodiments the pH of such pharmaceutical composition is about 5.

In certain embodiments such pharmaceutical composition is a suspension formulation.

In certain embodiments such pharmaceutical is a dry composition. It is understood that such dry composition may be obtained by drying, such as lyophilizing, a suspension composition.

If the pharmaceutical composition is a parenteral composition, suitable excipients may be categorized as, for example, buffering agents, isotonicity modifiers, preservatives, stabilizers, anti-adsorption agents, oxidation protection agents, viscosifiers/viscosity enhancing agents, anti-agglomeration agents or other auxiliary agents. However, in some cases, one excipient may have dual or triple functions. Excipient may be selected from the group consisting of

(i) Buffering agents: physiologically tolerated buffers to maintain pH in a desired range, such as sodium phosphate, bicarbonate, succinate, histidine, citrate and acetate, sulphate, nitrate, chloride, pyruvate; antacids such as Mg(OH)₂ or ZnCO₃ may be also used;

(ii) Isotonicity modifiers: to minimize pain that can result from cell damage due to osmotic pressure differences at the injection depot; glycerin and sodium chloride are examples; effective concentrations can be determined by osmometry using an assumed osmolality of 285-315 mOsmol/kg for serum;

(iii) Preservatives and/or antimicrobials: multidose parenteral formulations require the addition of preservatives at a sufficient concentration to minimize risk of patients becoming infected upon injection and corresponding regulatory requirements have been established; typical preservatives include m-cresol, phenol, methylparaben, ethylparaben, propylparaben, butylparaben, chlorobutanol, benzyl alcohol, phenylmercuric nitrate, thimerosol, sorbic acid, potassium sorbate, benzoic acid, chlorocresol, and benzalkonium chloride;

(iv) Stabilizers: Stabilisation is achieved by strengthening of the protein-stabilising forces, by destabilisation of the denatured state, or by direct binding of excipients to the protein; stabilizers may be amino acids such as alanine, arginine, aspartic acid, glycine, histidine, lysine, proline, sugars such as glucose, sucrose, trehalose, polyols such as glycerol, mannitol, sorbitol, salts such as potassium phosphate, sodium sulphate, chelating agents such as EDTA, hexaphosphate, ligands such as divalent metal ions (zinc, calcium, etc.), other salts or organic molecules such as phenolic derivatives; in addition, oligomers or polymers such as cyclodextrins, dextran, dendrimers, PEG or PVP or protamine or HS A may be used;

(v) Anti-adsorption agents: Mainly ionic or non-ionic surfactants or other proteins or soluble polymers are used to coat or adsorb competitively to the inner surface of the formulation's container; e.g., poloxamer (Pluronic F-68), PEG dodecyl ether (Brij 35), polysorbate 20 and 80, dextran, polyethylene glycol, PEG-polyhistidine, BSA and EISA and gelatins; chosen concentration and type of excipient depends on the effect to be avoided but typically a monolayer of surfactant is formed at the interface just above the CMC value;

(vi) Oxidation protection agents: antioxidants such as ascorbic acid, ectoine, methionine, glutathione, monothioglycerol, morin, polyethylenimine (PEI), propyl gallate, and vitamin E; chelating agents such as citric acid, EDTA, hexaphosphate, and thioglycolic acid may also be used;

(vii) Viscosifiers or viscosity enhancers: retard settling of the particles in the vial and syringe and are used in order to facilitate mixing and resuspension of the particles and to make the suspension easier to inject (i.e., low force on the syringe plunger); suitable viscosifiers or viscosity enhancers are, for example, carbomer viscosifiers like Carbopol 940, Carbopol Ultrez 10, cellulose derivatives like hydroxypropylmethylcellulose (hypromellose, EIPMC) or diethylaminoethyl cellulose (DEAE or DEAE-C), colloidal magnesium silicate (Veegum) or sodium silicate, hydroxyapatite gel, tricalcium phosphate gel, xanthans, carrageenans like Satia gum UTC 30, aliphatic poly(hydroxy acids), such as poly(D,L- or L-lactic acid) (PLA) and poly(glycolic acid) (PGA) and their copolymers (PLGA), terpolymers of D,L-lactide, glycolide and caprolactone, poloxamers, hydrophilic poly(oxyethylene) blocks and hydrophobic poly(oxypropylene) blocks to make up a triblock of poly(oxyethylene)-poly(oxypropylene)-poly(oxyethylene) (e.g. Pluronic®), polyetherester copolymer, such as a polyethylene glycol terephthalate/polybutylene terephthalate copolymer, sucrose acetate isobutyrate (SAIB), dextran or derivatives thereof, combinations of dextrans and PEG, polydimethylsiloxane, collagen, chitosan, polyvinyl alcohol (PVA) and derivatives, polyalkylimides, poly (acrylamide-co-diallyldimethyl ammonium (DADMA)), polyvinylpyrrolidone (PVP), glycosaminoglycans (GAGs) such as dermatan sulfate, chondroitin sulfate, keratan sulfate, heparin, heparan sulfate, hyaluronan, ABA triblock or AB block copolymers composed of hydrophobic A-blocks, such as polylactide (PLA) or poly(lactide-co-glycolide) (PLGA), and hydrophilic B-blocks, such as polyethylene glycol (PEG) or polyvinyl pyrrolidone; such block copolymers as well as the abovementioned poloxamers may exhibit reverse thermal gelation behavior (fluid state at room temperature to facilitate administration and gel state above sol-gel transition temperature at body temperature after injection);

(viii) Spreading or diffusing agent: modifies the permeability of connective tissue through the hydrolysis of components of the extracellular matrix in the intrastitial space such as but not limited to hyaluronic acid, a polysaccharide found in the intercellular space of connective tissue; a spreading agent such as but not limited to hyaluronidase temporarily decreases the viscosity of the extracellular matrix and promotes diffusion of injected drugs;

(ix) anti-agglomeration agents, such as propylene glycol; and

(x) Other auxiliary agents: such as wetting agents, viscosity modifiers, antibiotics, hyaluronidase; acids and bases such as hydrochloric acid and sodium hydroxide are auxiliary agents necessary for pH adjustment during manufacture.

In a further aspect the present invention relates to the use of the conjugates of the present invention or the pharmaceutical compositions comprising said conjugates as a medicament.

If -D is an antibiotic moiety said medicament is an antibiotic.

In a further aspect the present invention relates to the conjugates of the present invention or the pharmaceutical compositions comprising said conjugates for use in the diagnosis, prophylaxis or treatment of a disease that can be treated with the conjugates of the present invention.

If -D is an antibiotic moiety the disease that can be treated may be an infection, such as an infection in a body compartment, in particular a joint infection, such as a joint infection related to surgical implants.

In certain embodiments the infection is in a body compartment. Such body compartment may be selected from the group consisting of body cavities, body spaces, brain or parts thereof, ear or parts thereof, nose, throat, sinuses, lung or parts thereof, abdomen, bone, skin, muscle, abscess, small intestine, large intestine, cyst, uterus, amniotic sac and joint.

In certain embodiments such body compartment is any cavity of the human body, such as the oral cavity, cranial cavity, spinal cavity, dorsal cavity, thoracic cavity, pericardial cavity, abdominal cavity, ventral cavity, retroperitoneal space, abdominopelvic cavity, pelvic cavity and its enclosed organs.

In certain embodiments the body compartment is selected from the group consisting of the retropharyngeal space, retropalatial space, mediastinal space, retrosternal space, pleural space, retroperitoneal space, prevesical space, paravesical space, vesicocervical space, rectovaginal space, pararectal space, presacral space, subphrenic space, subhepatic space, supramesocolic space and inframesocolic space.

In certain embodiments the body compartment is the brain or one or more parts of it.

In certain embodiments the body compartment is the ear or one or more parts of it, such as the middle ear.

In certain embodiments the body compartment is the nose, throat, and sinuses.

In certain embodiments the body compartment is the lung or parts of it.

In certain embodiments the body compartment is the abdomen.

In certain embodiments the body compartment is bone, such as the pelvis.

In certain embodiments the body compartment is the skin.

In certain embodiments the body compartment is muscles.

In certain embodiments the body compartment is an abscess.

In certain embodiments the body compartment is the small intestine, such as the duodenum, ileum and jejunum.

In certain embodiments the body compartment is the large intestine, such as the colon, appendix and rectum.

In certain embodiments the body compartment is a cyst.

In certain embodiments the body compartment is the uterus.

In certain embodiments the body compartment is the amniotic sac.

In certain embodiments the body compartment is a joint.

If the infection is in a joint, the conjugate of the present invention may be administered via intraarticular injection.

In certain embodiments a single injection into the intra-articular compartment of at least one conjugate of the present invention provides a concentration of at least 1 μg antibiotic/ml synovial fluid for at least 3 days, such as for 3 days, 4 days or 5 days.

In certain embodiments a single injection into the intra-articular compartment of at least one conjugate of the present invention provides a concentration of at least 5 μg antibiotic/ml synovial fluid for at least 3 days, such as for 3 days, 4 days or 5 days.

In certain embodiments a single injection into the intra-articular compartment of at least one conjugate of the present invention provides a concentration of at least 25 μg antibiotic/ml synovial fluid for at least 3 days, such as for 3 days, 4 days or 5 days.

In certain embodiments a single injection into the intra-articular compartment of at least one conjugate of the present invention provides a concentration of at least 50 μg antibiotic/ml synovial fluid for at least 3 days, such as for 3 days, 4 days or 5 days.

In certain embodiments a single injection into the intra-articular compartment of at least one conjugate of the present invention provides a concentration of at least 75 μg antibiotic/ml synovial fluid for at least 3 days, such as for 3 days, 4 days or 5 days.

In certain embodiments a single injection into the intra-articular compartment of at least one conjugate of the present invention provides a concentration of at least 100 μg antibiotic/ml synovial fluid for at least 3 days, such as for 3 days, 4 days or 5 days.

In certain embodiments a single injection into the intra-articular compartment of at least one conjugate of the present invention provides a concentration of at least 150 μg antibiotic/ml synovial fluid for at least 3 days, such as for 3 days, 4 days or 5 days.

In certain embodiments a single injection into the intra-articular compartment of at least one conjugate of the present invention provides a concentration of at least 200 μg antibiotic/ml synovial fluid for at least 3 days, such as for 3 days, 4 days or 5 days.

In certain embodiments a single injection into the intra-articular compartment of at least one conjugate of the present invention provides a concentration of at least 250 μg antibiotic/ml synovial fluid for at least 3 days, such as for 3 days, 4 days or 5 days.

In certain embodiments a single injection into the intra-articular compartment of at least one conjugate of the present invention provides a concentration of at least 300 μg antibiotic/ml synovial fluid for at least 3 days, such as for 3 days, 4 days or 5 days.

In certain embodiments a single injection into the intra-articular compartment of at least one conjugate of the present invention provides a concentration of at least 400 μg antibiotic/ml synovial fluid for at least 3 days, such as for 3 days, 4 days or 5 days.

In certain embodiments a single injection into the intra-articular compartment of at least one conjugate of the present invention provides a concentration of at least 500 μg antibiotic/ml synovial fluid for at least 3 days, such as for 3 days, 4 days or 5 days.

In certain embodiments the antibiotic moieties released from the conjugate after a single intra-articular injection provide a concentration of said antibiotic in the intra-articular compartment that is at least 1.1-fold above the minimal biofilm eradicating concentration of the respective antibiotic for at least 3 days, such as for at least 4 days, at least 5 days, at least 6 days or at least 7 days.

In certain embodiments the antibiotic moieties released from the conjugate after a single intra-articular injection provide a concentration of said antibiotic in the intra-articular compartment that is at least 1.2-fold above the minimal biofilm eradicating concentration of the respective antibiotic for at least 3 days, such as for at least 4 days, at least 5 days, at least 6 days or at least 7 days.

In certain embodiments the antibiotic moieties released from the conjugate after a single intra-articular injection provide a concentration of said antibiotic in the intra-articular compartment that is at least 1.3-fold above the minimal biofilm eradicating concentration of the respective antibiotic for at least 3 days, such as for at least 4 days, at least 5 days, at least 6 days or at least 7 days.

In certain embodiments the antibiotic moieties released from the conjugate after a single intra-articular injection provide a concentration of said antibiotic in the intra-articular compartment that is at least 1.4-fold above the minimal biofilm eradicating concentration of the respective antibiotic for at least 3 days, such as for at least 4 days, at least 5 days, at least 6 days or at least 7 days.

In certain embodiments the antibiotic moieties released from the conjugate after a single intra-articular injection provide a concentration of said antibiotic in the intra-articular compartment that is at least 1.5-fold above the minimal biofilm eradicating concentration of the respective antibiotic for at least 3 days, such as for at least 4 days, at least 5 days, at least 6 days or at least 7 days.

In certain embodiments the antibiotic moieties released from the conjugate after a single intra-articular injection provide a concentration of said antibiotic in the intra-articular compartment that is at least 1.6-fold above the minimal biofilm eradicating concentration of the respective antibiotic for at least 3 days, such as for at least 4 days, at least 5 days, at least 6 days or at least 7 days.

In certain embodiments the antibiotic moieties released from the conjugate after a single intra-articular injection provide a concentration of said antibiotic in the intra-articular compartment that is at least 1.7-fold above the minimal biofilm eradicating concentration of the respective antibiotic for at least 3 days, such as for at least 4 days, at least 5 days, at least 6 days or at least 7 days.

In certain embodiments the joint of a joint infection may be a synovial joint. Such synovial joint may be selected from the group consisting of hinge joints and ball and socket joints. In certain embodiments the joint is a hinge joint. In certain embodiments the joint is a ball and socket joint.

Examples for a synovial joint are knee, hip, shoulder, elbow, foot, hand, sternoclavicular joint and vertebral articulations.

Examples for a joint of the knee are tibiofemoral joint and patellofemoral joint.

Examples for a joint of the shoulder are glenohumeral joint and acromioclavicular joint.

Examples for a joint of the elbow are humero-ulnar joint, humero-radial joint and radio-ulnar joint.

It is understood that the term “joints of the foot” also covers joints of the toes. Examples for a joint of the foot are ankle, subtalar and talocalcaneal joint.

It is understood that the term “joints of the hand” also covers joints of the fingers. Example for a joint of the hand are wrist, intercarpal joint, midcarpal joint, carpometacarpal joint and metacarpophalangeal joint.

Examples for a vertebral articulation are zygapophyseal joints, temporomandibular joints and sacroiliac joints.

In certain embodiments the joint is selected from the group consisting of knee, hip, shoulder, elbow and ankle. In certain embodiments the joint is a knee. In certain embodiments the joint is a hip. In certain embodiments the joint is a shoulder.

In certain embodiments the infection, such as an infection in a body compartment, such as a joint infection, is an infection related to a surgical implant.

Examples for such surgical implant are pins, rods, screws, artificial joints, mesh, clips, sutures, wires, tubes, catheters, pumps, filters, prostheses, plates, fasteners, washers, bolts, seeds, beads, staples, nails, shunts, cuffs, buttons, ports, cement, fixators, stents, fillers, wax, wraps, weights, stimulators, anchors, expanders, guidewires, fillers, polymers, film, fixators, drains, lines and cones.

In certain embodiments the surgical implant is an artificial joint. In certain embodiments the surgical implant is a prosthesis.

In certain embodiments surgical implants are at least partially made from one or more material selected from the group consisting of metals, ceramics, natural polymers, artificial polymers, bone cement, foreign organic material, artificial tissue and natural tissue. Such natural tissue may be selected from the group consisting of ligament, skin, muscle and bone. In certain embodiments the natural tissue is bone.

In certain embodiments the conjugate or the pharmaceutically acceptable salt thereof or the pharmaceutical composition comprising said conjugate or its pharmaceutically acceptable salt are for use in a method of preventing an infection, such as an infection in a body compartment, such as in a joint, and in particular a joint infection related to a surgical implant.

In such case the conjugate of the present invention or the pharmaceutically acceptable salt thereof or the pharmaceutical composition comprising said conjugate or its pharmaceutically acceptable salt may be administered at or close to the infection site prior, during or after the implantation of the surgical implant. In certain embodiments it is administered prior to the implantation a surgical implant. In certain embodiments it is administered during the implantation of a surgical implant. In certain embodiments it is administered after the implantation of a surgical implant, such as for example no more than 1 hour after the implantation, no later than 2 hours after the implantation, no later than 5 hours after the implantation, no later than 10 hours after the implantation, no later than 24 hours after the implantation, no later than 48 hours after the implantation or no later than 72 hours after the implantation, no later than 96 hours after the implantation, no later than a week after the implantation, no later than two weeks after the implantation, no later than three weeks after the implantation, no later than four weeks after the implantation, no later than six weeks after the implantation or no later than eight weeks after the implantation. In certain embodiments it may be administered later than two months after the implantation.

In certain embodiments the conjugate or the pharmaceutically acceptable salt thereof or the pharmaceutical composition comprising said conjugate or its pharmaceutically acceptable salt are for use in a method of treating an infection, such as an infection in a body compartment, such as a in a joint, and in particular a joint infection related to a surgical implant.

In certain embodiments the infection comprises the presence of a biofilm in said infected compartment, in particular a biofilm on at least one surface of a surgical implant. Such biofilm may comprise organisms selected from the group consisting of bacteria, mycobacteria and fungi. Accordingly, in certain embodiments the method of preventing or treating a joint infection also comprises the prevention of biofilm formation or the eradication of an existing biofilm.

In certain embodiments such biofilm comprises bacteria. Such bacteria may be gram-positive or gram-negative. They may be aerobic or anaerobic bacteria. In certain embodiments the biofilm comprises multiple different species. In certain embodiments the biofilm comprises one predominant species, to which at least 80%, such as at least 85%, at least 90%, at least 92%, at least 95%, at least 96%, at least 97%, at least 98% or at least 99%, of all bacteria present in the biofilm belong.

Examples for gram-positive bacteria are Staphylococcus, Streptococcus, Enterococcus, 10 Clostridium, Bacillus, Listeria and lactic acid bacteria, such as Staphylococcus aureus, Staphylococcus epidermidis, Staphylococcus saprophyticus, Streptococcus pneumoniae, Streptococcus pyogenes, Streptococcus agalactiae, Streptococcus dysgalactiae, Streptococcus viridans, Enterococcus faecalis, Enterococcus faeeium, Clostridium tetani, Clostridium botulinum, Clostridium perfringes, Clostridium difficile, Bacillus anthracis, Listeria monocytogenes and Propionibacterium acnes.

Examples for gram-negative bacteria are Enterobacteriaceae, Vibrionaceae, Pseudomonadaceae, Baeteroidaceae, Actinomyces, Neisseria, Hemophilus, Bordetella, Legionella, Treponema, Borrelia, Chlamydia, Rickettsia, Ehrlichia, Mycoplasma and Burkholderia, such as Salmonella species, Shigella dysenteriae, Klebsiella pneumoniae, Escherichia coli, Escherichia faecalis, Vibrio cholera, Campylobacter jejuni, Pseudomonas aeruginosa, Baeteroides fragilis, Neisseria meningitidis, Neisseria gonorrhoeae, Haemophilus influenza, Actinomyces isrealli. Mycoplasma pneumoniae, Acinetobacter baumanii, Citrobacter, Achromobacter and Stenotrophomonas.

In certain embodiments the biofilm comprises mycobacteria.

In certain embodiments the biofilm comprises fungi. Such fungi may be molds or yeasts.

Examples for fungi are Candida, Aspergillus, Cryptococcus, Trichosporon, Coccidioides, and Pneumocystis, such as Candida albicans, Candida parapsilosis, Candida tropicalis, Candida parapsilosis, Candida glabrata; Aspergillus fumigatus, Coccioides immitis, Coccioides neoformans, Trichosporon asahii, and Pneumocystis carinii.

In a further aspect the present invention relates to a method of treating a patient suffering from a disease that can be treated with D-H or D-OH comprising administering an effective amount of the conjugate of the present invention or the pharmaceutical compositions comprising said conjugates to the patient.

If D-H or D-OH is an antibiotic the disease that can be treated is preferably an infection, such as a joint infection, such as a joint infection related to surgical implants, as described above.

### EXAMPLES

### Materials and Methods

All materials were commercially available except where stated otherwise.

RP-HPLC purification:

For preparative RP-HPLC a Waters 600 controller and a 2487 Dual Absorbance Detector was used, equipped with the following column: Waters XBridge™ BEH300 Prep C₁₋₈ 10 μm, 150×30 mm, flow rate 40 mL/min. Gradients of solvent system A (water containing 0.1% TFA v/v) and solvent system B (acetonitrile containing 0.1% TFA v/v) were used. Products were detected at 215 nm. HPLC fractions containing product were pooled and lyophilized if not stated otherwise.

Flash Chromatography:

Flash chromatography purifications were performed on an Isolera One system or an Isolera Four system from Biotage AB, Sweden, using Biotage KP-Sil silica cartridges and CH₂C₁₋₂/MeOH, CH₂Cl₂/ACN, CH₂Cl₂/THF, n-heptane/ethyl acetate or n-heptane/methyl acetate as eluents. Products were detected at 215 nm, 254 nm or 280 nm.

RP-LPLC purification:

Low pressure RP chromatography purifications were performed on an Isolera One system or an Isolera Four system from Biotage AB, Sweden, using Biotage SNAP C18 cartridges. Gradients of solvent system A (water containing 0.1% TFA v/v) and solvent system B (acetonitrile containing 0.1% TFA v/v) were used. Products were detected at 215 nm. LPLC fractions containing product were pooled and lyophilized if not stated otherwise.

### Analytical Methods

UPLC-MS analysis:

Analytical ultra-performance LC (UPLC)-MS was performed on a Waters Acquity system or an Agilent 1290 Infinity II equipped with a Waters BEH300 C18 column (2.1×50 mm, 1.7 μm particle size or 2.1×100 mm, 1.7 μm particle size; solvent A: water containing 0.04% TFA (v/v), solvent B: acetonitrile containing 0.05% TFA (v/v) or solvent A: water containing 0.1% FA (v/v), solvent B: acetonitrile containing 0.1% FA (v/v)) coupled to an LTQ Orbitrap Discovery mass spectrometer from Thermo Scientific or coupled to a Waters Micromass ZQ or coupled to Single Quad MS System from Agilent or coupled to an Agilent Triple Quad 6460 system.

SEC analysis:

Size-exclusion chromatography (SEC) was performed on an Agilent 1260 system, equipped with a Sepax Zenix SEC-150 column (150 Å, 7.8×300 mm; isocratic: 60:40 v/v mixture of water containing 0.05% TFA and acetonitrile containing 0.04% TFA) with detection at 215 nm and 280 nm.

Amine content determination on the PEG-hydrogel beads:

Amino group content of the PEG-hydrogel was determined by conjugation of an Fmoc-amino acid to the free amino groups on the hydrogel and subsequent Fmoc-determination as described by Gude, M., J. Ryf, et al. (2002) Letters in Peptide Science 9(4): 203-206.

Maleimide content determination on the PEG-hydrogel beads:

Maleimide group content of the PEG-hydrogel was determined by conjugation of Fmoc-cysteine to the maleimide residues on the hydrogel and subsequent Fmoc-determination following a procedure, which is based on Gude, M., Ryf, J. et al. (2002) Letters in Peptide Science 9(4): 203-206 and Smyth, D. G., Blumenfeld, O. O., Konigsberg, W. (1964) Biochemical Journal 91: 589.

### Quantitative Amino Acid Analysis (QAAA):

Quantitative amino acid analysis was performed to determine the amount of daptomycin in a sample matrix with unknown content. For the content determination, a material sample containing daptomycin was hydrolysed using a TFA/HCl mixture and microwave irradiation. The resulting single amino acids was dye labelled and analysed chromatographically. The contents of aspartic acid, alanine and ornithine were calculated using calibration curves of the respective amino acid standards. The amount of daptomycin was calculated using the averaged content values of aspartic acid, alanine and ornithine.

### Hydrogel Degradation Kinetics:

A hydrogel sample was incubated with degradation buffer of the desired pH in a water bath at the desired temperature. For each sampling time-point, the reaction mixture was homogenized, centrifuged, supernatant was withdrawn, filtered through a syringe filter and transferred into a sterile Eppendorf tube. Samples were further incubated at the same temperature. At the end of the incubation time, all samples were quenched with acetic acid, and analysed chromatographically. The obtained peak areas of the individual samples were used to calculate degradation kinetics.

### Example 1

### Synthesis of Linker Reagent 1f

Tinker reagent 1f was synthesized according to the following scheme:

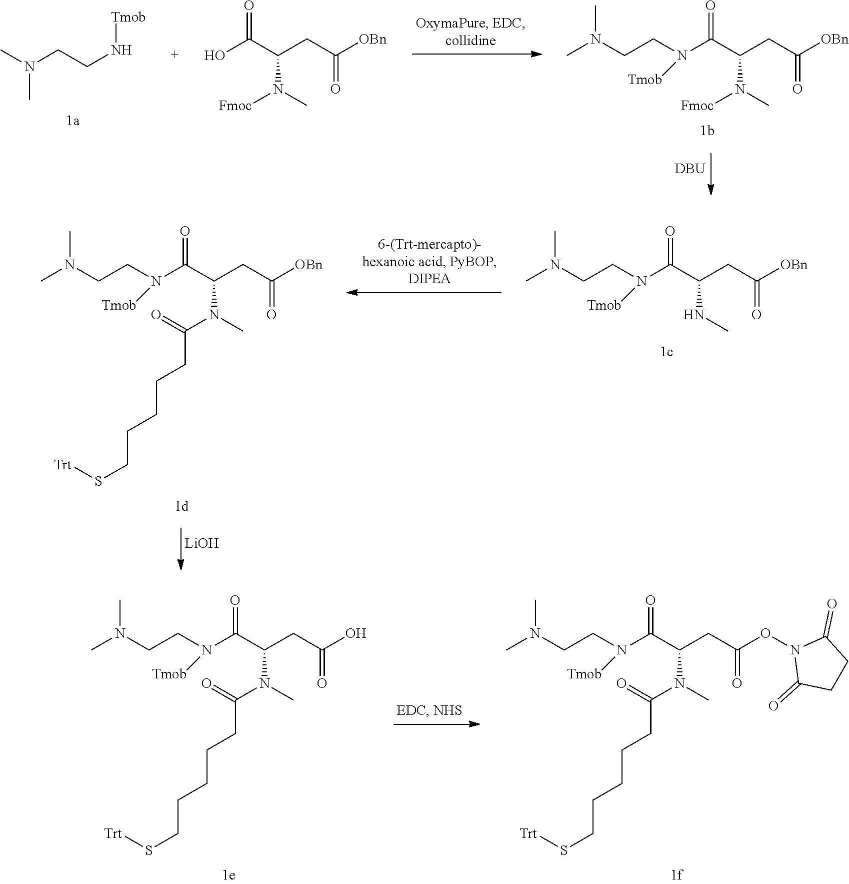

To a solution of N,N′-di methylethylenediamine (2.00 g, 22.69 mmol) and NaCNBH₃ (1.35 g, 21.55 mmol) in MeOH (40 mL) was added 2,4,6-trimethoxybenzaldehyde (4.23 g, 21.55 mmol) over two hours. After complete addition, the mixture was stirred at r.t. for 1 hour, acidified with 1 M HCl (60 mL) and stirred for further 30 min. To the reaction mixture saturated NaHCO₃ solution (70 mL) was added and the solution was extracted with CH₂Cl₂ (5×150 mL). The combined organic phases were dried over Na₂SO₄, filtered and the solvents were evaporated in vacuo. The resulting N,N-dimethyl-N′-Tmob-ethylenediamine 1a was dried in high vacuum and used in the next reaction step without further purification.

To a solution of Fmoc-A-Me-Asp(OBn)-OH (4.63 g, 10.07 mmol) in CH₂Cl₂ (108 mL) EDC (2.51 g, 13.09 mmol), OxymaPure® (2.00 g, 14.09 mmol) and 2,4,6-collidine (2.53 mL, 2.32 g, 19.13 mmol) were added and the mixture was stirred for 5 min. A solution of crude 1a (3.00 g, max. 11.18 mmol) in CH₂Cl₂ (27 mL) was added and the solution was stirred at r.t. for 1 hour. The reaction was quenched by addition of 0.1 M HCl (300 mL) and the acidified mixture was extracted with CH₂Cl₂ (5×40 mL). The combined organic layers were washed with saturated NaHCQ₃ solution (2×90 mL). The organic phase was dried over Na₂SQ₄, filtered and the solvent was evaporated in vacuo. Crude 1b was purified by flash chromatography.

Yield: 5.31 g (7.48 mmol, 74% over two steps)

MS: m/z 710.23=[M+H]⁺, (calculated monoisotopic mass: [M]=709.34.)

To a solution of 1b (5.31 g, 7.48 mmol) in THF (53 mL) DBU (1.31 mL, 1.33 g, 8.75 mmol) was added and the solution was stirred at r.t. for 12 min. The reaction mixture was submitted to flash chromatography and 1c was isolated from the product fractions by evaporation of the solvents in vacuo.

Yield: 3.16 g (6.48 mmol, 87%)

MS: m/z 488.13=[M+H]⁺, (calculated monoisotopic mass: [M]=487.27.)

To a solution of 1c (3.16 g, 6.48 mmol), PyBOP (4.05 g, 7.78 mmol) and DIPEA (3.39 mL, 2.51 g, 19.44 mmol) in CH₂Cl₂ (32 mL), a solution of 6-tritylmercaptohexanoic acid (3.04 g, 7.78 mmol) in CH₂Cl₂ (32 mL) was added and the mixture was stirred for 24 hours. Additional 6-tritylmercaptohexanoic acid (633 mg, 1.62 mmol) and PyBOP (843 mg, 1.62 mmol) were added and the mixture was stirred for additional 5 hours. After dilution with CH₂Cl₂ (600 mL), the organic layer was washed with 0.1 M HCl (3×300 mL) and brine (300 mL), dried over Na₂SO₄, filtered and the solvent was evaporated in vacuo. Crude Id was purified by flash chromatography.

Yield: 5.06 g (5.88 mmol, 91%)

MS: m/z 860.45=[M+H]⁺, (calculated monoisotopic mass: [M]=859.42.)

To a solution of Id in a mixture of THF (61 mL) and water (61 mL) LiOH (423 mg, 17.66 mmol) was added and the solution was stirred at r.t. for six hours. After dilution with CH₂Cl₂ (500 mL), the organic layer was washed with a mixture of 0.1 M HCl/brine (1:1 v/v, 3×300 mL). The aqueous layers were re-extracted with CH₂Cl₂ (5×100 mL). The combined organic layers were washed with brine (200 mL), dried over Na₂SO₄, filtered and the solvents were evaporated in vacuo. Crude 1e was dried in high vacuum and used without further purification in the next step.

To a solution of crude 1e (5.05 g, max. 6.56 mmol) in CH₂Cl₂ (60 mL), NHS (1.13 g, 9.85 mmol) and EDC (1.89 g, 9.85 mmol) were added and the mixture was stirred at r.t. for 130 min. After evaporation of the solvent in vacuo, the residue was dissolved in a mixture of MeCN/water/TFA (8:2:0.002 v/v, 10 mL) and the resulting solution was purified by automated RP-LPLC to yield pure 1f after lyophilization.

Yield: 4.15 g (4.52 mmol, 76%, 96% purity by UV215)

MS: m/z 867.44=[M+H]⁺, (calculated monoisotopic mass: [M]=866.39.)

### Example 2

### Synthesis of Daptomycin Linker Thiol 2b

Daptomycin linker thiol 2b was synthesized according to the following scheme:

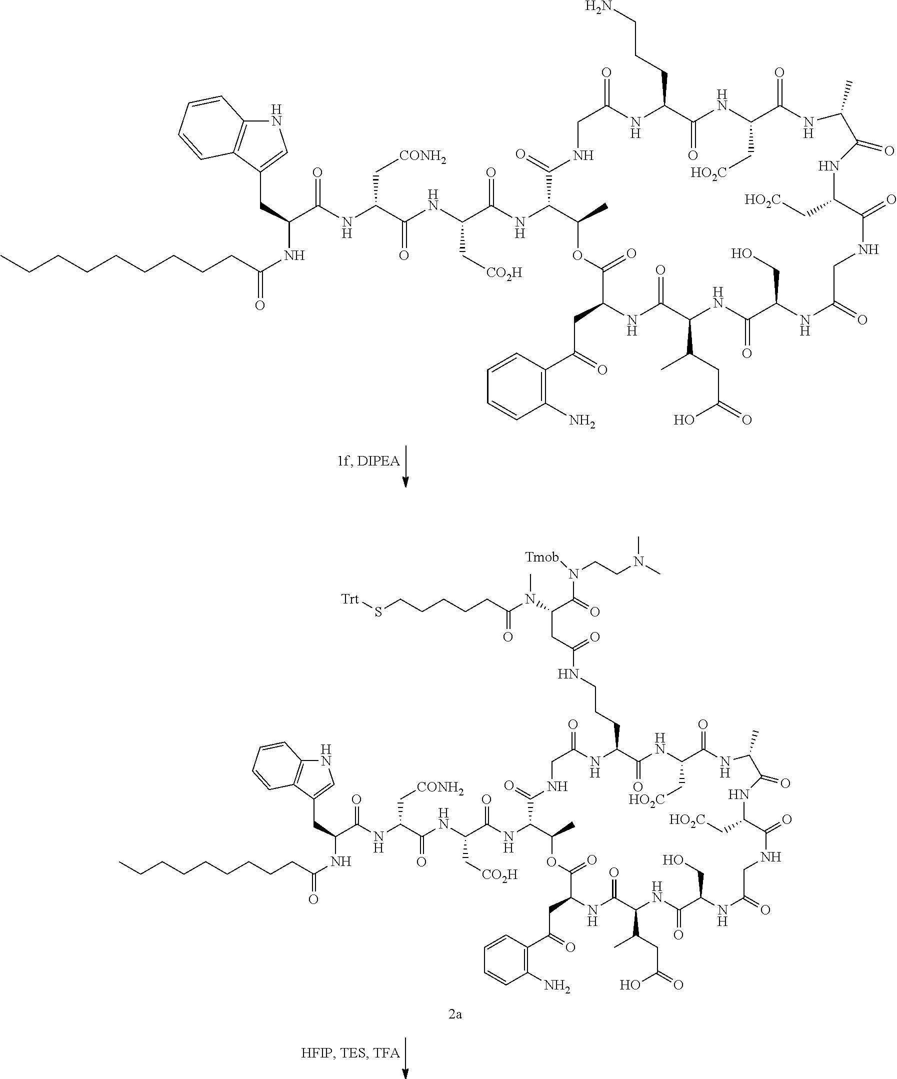

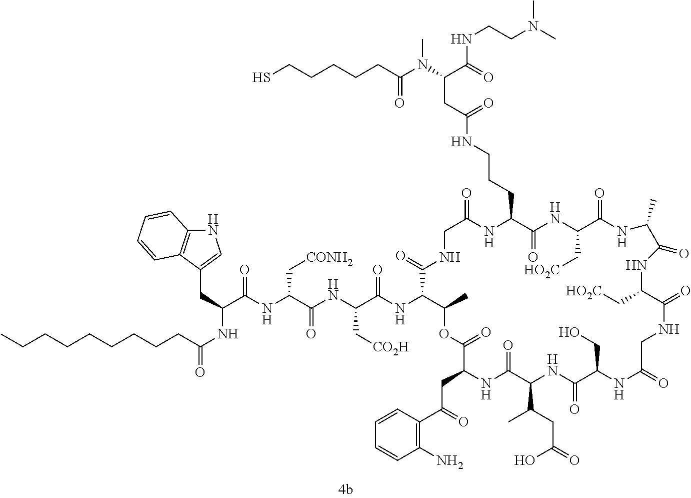 [SMILES: CCCCCCCCCC(=O)N[C@@H](Cc1c[nH]c2ccccc12)C(=O)N[C@H](CC(N)=O)C(=O)N[C@@H](CC(=O)O)C(=O)N[C@@H]1C(=O)NCC(=O)N[C@@H](CCCNC(=O)C[C@@H](C(=O)NCCN(C)C)N(C)C(=O)CCCCCS)C(=O)N[C@@H](CC(=O)O)C(=O)N[C@H](C)C(=O)N[C@@H](CC(=O)O)C(=O)NCC(=O)N[C@H](CO)C(=O)N[C@@H](C(C)CC(=O)O)C(=O)N[C@@H](CC(=O)c2ccccc2N)C(=O)O[C@@H]1C]

To a mixture of daptomycin (1.08 g, approx. 0.63 mmol) and 1f (0.99 g, 1.01 mmol) in DMSO (38 mL) DIPEA (0.97 mL, 0.72 g, 5.69 mmol) was added and it was stirred for 380 min. After quenching with TFA (0.44 mL, 0.66 g, 5.69 mmol), the mixture was added to MTBE in 50 mL Falcon tubes (1 mL solution and 40 mL MTBE per tube) to precipitate the conjugate. The tubes were shaken and centrifuged. After decanting the supernatants, the residues were combined and dried in high vacuum overnight. Crude 2a was used for the next step without further purification.

Crude 2a (2.50 g, max. 0.63 mmol) was dissolved in a mixture of HFIP/TES (39:1 v/v, 57 mL) and the solution was stirred at r.t. for 5 min. TFA (4.01 mL) was added and the reaction mixture was stirred at r.t. for two hours. All volatiles were removed in vacuo and the residue was dissolved in a mixture of DCM/TFA (98:2 v/v, 3.0 mL). The solution was added to MTBE in 50 mL Falcon tubes (1 mL solution and 40 mL MTBE per tube) to precipitate the material. The tubes were shaken and centrifuged. After decanting the supernatants, the combined residues were dried in high vacuum overnight. Crude 2b was purified by RP-LPLC to afford pure and mixed product fractions. Pure product fractions were lyophilized to afford a first crop of pure linker thiol. The mixed fractions were additionally purified by preparative RP-HPLC to afford a second crop of pure linker thiol. Both product batches were combined to afford pure 2b.

Yield: 1.00 g (0.46 mmol, 72%, 99% purity at 215 nm)

MS: m/z 975.92=[M+2H]²⁺, (calculated monoisotopic mass: [M]=1948.89.)

### Example 3

### Synthesis of Cross-Linker Reagent 3d

Cross-linker reagent 3d was synthesized according to the following scheme. Theoretical calculations of the Mw of the polydisperse PEG conjugates were exemplarily performed for a PEG 1000 with 23 ethylene glycol units that has a Mw of 1031.22 g/mol (exact mass: 1030.61 g/mol):

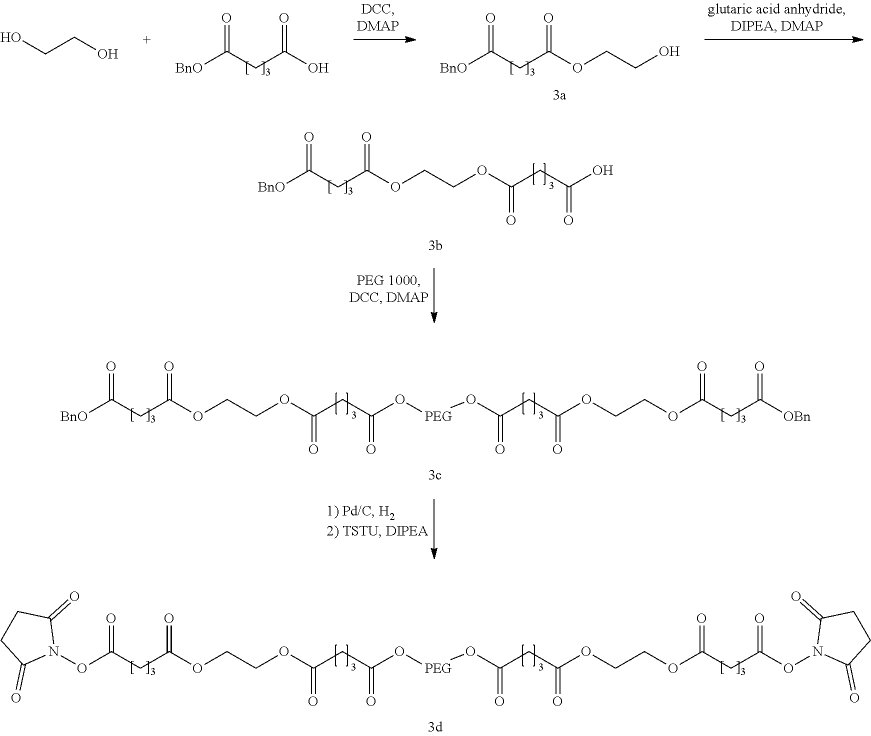

Glutaric acid monobenzyl ester (40.0 g, 180 mmol), ethylene glycol (101 mL, 1.80 mol) and DMAP (2.20 g; 18.0 mmol) were dissolved in CH₂Cl₂ (400 mL). DCC (44.6 g, 216 mmol) was added to the solution, and the mixture was stirred at room temperature for one hour. The reaction mixture was filtered and the filter cake was washed with additional CH₂Cl₂ (50 mL). The filtrate was washed with 0.1 N hydrochloric acid (2×250 mL) and brine (1×250 mL). The organic phase was dried over MgSO₄, filtered and all volatiles were evaporated in vacuo.

The residue was purified by flash chromatography to afford intermediate 3a.

Yield: 41.9 g (157 mmol, 87%)

MS: m/z 267.00=[M+H]⁺, (calculated monoisotopic mass: [M]=266.16.)

Intermediate 3a (41.0 g, 154 mmol), glutaric acid anhydride (31.6 g, 277 mmol) and DMAP (3.76 g, 30.8 mmol) were dissolved in CH₂Cl₂ (164 mL). DIPEA, (53.8 mL, 308 mmol) was added and the mixture was stirred at r.t. for two hours. The mixture was washed with 1 M hydrochloric acid (1×400 mL, 1×200 mL) and brine (200 mL). The organic phase was dried over MgSO₄, filtered and all volatiles were evaporated in vacuo. The residue was purified by flash chromatography to afford intermediate 3b.

Yield: 34.9 g (91.7 mmol, 60%)

MS: m/z 381.05=[M+H]⁺, (calculated monoisotopic mass: [M]=380.15.)

Poly(ethylene glycol) (PEG 1000, 19.0 g), intermediate 3b (25.3 g, 66.5 mmol) and DMAP (116 mg, 0.95 mmol) were dissolved in CH₂Cl₂ (95 mL). DCC (13.7 g, 66.50 mmol) was added at 0° C. and the mixture was afterwards stirred at r.t. for 16 hours. The mixture was diluted with MTBE (95 mL), filtered and all volatiles of the filtrate were evaporated in vacuo. The residue was dissolved in CH₂Cl₂ (120 mL) and the solution was diluted with MTBE (1800 mL) and n-heptane (100 mL) and split in two halves. The mixtures were cooled to −20° C. for 20 h. The supernatants were decanted and the precipitates suspended in a −20° C. cold mixture of MTBE/n-heptane (9:1 v/v, 2× approx. 900 mL). The mixtures were stored at −20° C. for one hour before supernatants were decanted. The precipitates were again suspended in a −20° C. cold mixture of MTBE/n-heptane (9:1 v/v, 2× approx. 900 mL) and the resulting suspensions were combined and filtered. The filter cake was washed with a −20° C. cold mixture of MTBE/n-heptane (9:1 v/v, 500 mL) and was afterwards dried in high vacuum to afford pure intermediate 3c.

Yield: 28.2 g

MS: m/z 878.33=[M+2H]²⁺, (calculated monoisotopic mass: [M]=1754.89.) Compound 3c (28.1 g, 16.0 mmol) was dissolved in THE (281 mL) and palladium on charcoal (10% Pd, 0.68 g) was added. The reaction mixture was stirred at 50° C. under a hydrogen atmosphere for one hour. The mixture was filtered through a pad of Celite 503, which was flushed with additional THE (50 mL). To the combined filtrates, TSTU (19.3 g, 64.0 mmol) and DIPEA (11.2 mL, 64.0 mmol) were added and the reaction mixture was stirred at r.t. for three hours. The mixture was filtered and the filter cake was washed with THL (50 mL). All volatiles were removed from the combined filtrates in vacuo and the residue was dissolved in CH₂Cl₂ (1200 mL). The solution was washed with 0.5 M phosphate buffer pH 7.4 (2×600 mL) and brine (2×200 mL) and was afterwards dried over MgSO₄.

After filtration, all volatiles were removed in vacuo to afford crude NHS ester. The crude material was dissolved in toluene (1000 mL) and the solution was split in two halves. To each portion MTBE (450 mL) was added and the resulting mixtures were stored at −20° C. overnight. The supernatants were decanted and the solids were collected by filtration and washed with −20° C. cold MTBE (500 mL). The filter residue was transferred into a 100 mL flask and dried for 4 h in high vacuum. The residue was dissolved in CH₂Cl₂ (600 mL) and the solution was split in three portions. To each portion MTBE (800 mL) was added and the resulting mixtures were were stored at −20° C. overnight. The supernatants were decanted from the precipitated oils and all volatiles were removed. The residues were combined with the precipitated oils and the combined crude material was dissolved in THL (1200 mL) and the solution was split in four portions. To each portion MTBE (700 mL) was added and the resulting mixtures were were stored at −25° C. overnight. The supernatants were decanted and the solids were collected by filtration and washed with −20° C. cold MTBE (1000 mL). Pure cross-linker reagent 3d was obtained after drying in high vacuum.

Yield: 17.5 g

MS: m/z 885.25=[M+2H]²⁺, (calculated monoisotopic mass: [M]=1768.83.)

### Example 4

### Synthesis of Backbone Reagent 4

Backbone reagent 4 was synthesized as HCl salt using L-lysine building blocks, analogously to an earlier described procedure (WO2013/053856, example 1, compound 1g therein):

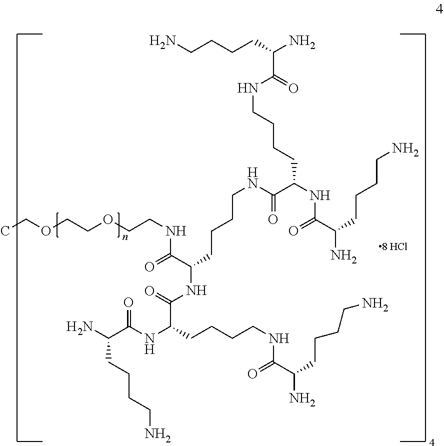

### Example 5

### Synthesis of PEG-Hydrogel Beads 5a, 5b, and 5c Containing Free Amino Groups

The weights of the PEG-hydrogel beads 5a, 5b and 5c were estimated by the volume of the aqueous hydrogel bead suspensions, calculating with 1 g of the dry PEG-hydrogel beads 5a, 5b or 5c swelling to a volume of approx. 20 mL under aqueous conditions. All liquids, solvents and reagent solutions were filtered through 0.2 μm PES filters (for aqueous solutions) or 0.2 μm PTFE filters (all others) before use.

A cylindrical 250 mL reactor with bottom outlet, diameter 60 mm, equipped with baffles, was charged with an emulsion of Cithrol™ DPHS (0.25 g) in heptane (75 mL). The reactor content was stirred with a pitch-blade stirrer, diameter 45 mm, at 520 rpm, at r.t. A solution of cross-linker 3d (3129 mg) and backbone reagent 4 (2600 mg) in DMSO (22.92 g) was added to the reactor and stirred for 10 min to form an emulsion. TMEDA (11.6 mL) was added to effect polymerization and the mixture was stirred at r.t. for 16 h. Acetic acid (17.8 mL) was added while stirring. After 10 min, a sodium chloride solution (15 wt %, 90 mL) was added under stirring. After 10 min, the stirrer was stopped and phases were allowed to separate. After 30 min, the aqueous phase containing the PEG-hydrogel beads was drained.

For bead size fractionation, the water-hydrogel suspension was diluted with ethanol (40 mL) and wet-sieved on 125, 100, 75, 63, and 50 μm (mesh opening) stainless steel sieves, diameter 200 mm using a sieving machine for 15 min. Sieving amplitude was 1.5 mm, liquid flow was 300 mL/min. First, a sodium chloride solution (20 wt %, 3000 mL), then water (1000 mL) was used as the liquid for wet-sieving. The bead fractions on the different sieves were transferred into 50 mL Falcon tubes (max. 14 mL bead suspension per tube) and successively washed with AcOH (0.1% v/v, 3× ˜40 mL) and ethanol (5-7× ˜40 mL) by addition, shaking, centrifugation and decantation. The bead fractions were transferred into 20 mL syringes with PE frits (max. ≈600 mg hydrogel beads per syringe) and dried in high vacuum for 16 hours to yield amine hydrogels 5a, 5b and 5c. The amine content of the hydrogels was determined for bead fraction 5a, representatively for all batches, by conjugation of an Fmoc-amino acid to the free amino groups on the hydrogel and subsequent Fmoc determination.

Yields: 5a (63 μm sieve fraction): ≈125 mg
5b (75 μm sieve fraction): ≈600 mg
5c (100 μm sieve fraction): ≈1400 mg

Amine content: 0.877 mmol/g

### Example 6

Synthesis of transient daptomycin-linker PEG-hydrogel conjugate 6b Amine hydrogel beads 5c (approx. 600 mg) were placed into a 20 mL syringe reactor with PE frit. The beads were washed with NMP (3×12 mL) and NMP/DIPEA (98:2 v/v, 2×12 mL) and all solvents were expelled afterwards. N-succinimidyl 3-maleimidopropionate (416 mg, 1.56 mmol) was dissolved in NMP (7.2 mL) and the resulting solution was drawn to the hydrogel in the syringe reactor. The suspension was allowed to incubate for two hours at r.t. under gentle agitation. The liquids were expelled and the hydrogel beads were washed with NMP (5×12 mL), AcOH (0.1% v/v, 5×12 mL) and ethanol (5×12 mL). Maleimide hydrogel 6a was obtained by drying in high vacuum for 5 days. The maleimide content of the functionalized PEG-hydrogel beads 6a was determined by conjugation of Fmoc-cysteine to the maleimide residues on the hydrogel and subsequent Fmoc determination.

Yield: not determined

Maleimide content: 0.7166 mmol/g

A suspension of the maleimide functionalized hydrogel beads 6a (346 mg, 0.248 mmol maleimides) in buffer (100 mM succinate, 0.05% Tween 20, pH 5.5, 15.0 mL) in a 50 mL Falcon tube was agitated for 5 min and then centrifuged. A part of the supernatant (approx. 11 mL) was discarded and a solution of daptomycin linker thiol 2b (820 mg, 0.376 mmol) in buffer (100 mM succinate, 0.05% Tween 20, pH 5.5, 32.8 mL) was added to the hydrogel suspension. The tube was agitated at r.t. and protected from light for 22 hours. The tube was centrifuged and the supernatant was partially removed to leave approx. 2 mL supernatant above the dense bead suspension. The beads were transferred into a 20 ml syringe reactor with a PE frit. The hydrogel beads were successively washed with buffer (100 mM succinate, 0.05% Tween 20, pH 5.5, 10×10 mF), AcOH (0.1% v/v, 10×10 mF), NMP/AcOH (97:3 v/v, 10×10 mL) and ethanol (10×10 mL). The transient daptomycin-linker PEG-hydrogel conjugate 6b was obtained after drying in high vacuum overnight. The daptomycin content of 6b was determined by QAAA.

Yield: 821 mg (99%, daptomycin content: 470.1 mg/g)

### Example 7

### Linker Release Kinetics for a Transient Daptomycin-Linker Hydrogel Conjugate

The linker kinetics with respect to the daptomycin species release from a transient daptomycin-linker hydrogel conjugate was investigated by incubation of transient daptomycin-linker PEG-conjugate 6b at pH 7.4 and 37° C. Daptomycin is prone to hydrolytic degradation and some minor different degradation pathways upon aqueous incubation. For determination of the linker kinetics on the carrier, the supernatant of the incubated suspension was analyzed by UPLC at 215 nm and all daptomycin-related peaks were taken into account for the calculation of the linker kinetics. The half-life of the linker with respect to daptomycin species release has been determined to be two weeks for the transient daptomycin-linker PEG-hydrogel conjugate 6b.

### Example 8

### Stability of Daptomycin in a Transient Daptomycin-Linker Hydrogel Conjugate

The relative stability of the covalently bound daptomycin in a transient daptomycin-linker hydrogel conjugate towards hydrolytic and other degradation pathways in comparison to free daptomycin was investigated. For that purpose, free daptomycin and transient daptomycin-linker PEG-conjugate 6b were incubated at pH 7.4 and 37° C. The supernatant of the carrier sample was exchanged five times within a week and the daptomycin purity in these samples was analyzed by UPLC. In parallel, analytical samples of the free daptomycin control solution were also analyzed by UPLC at the same incubation times. The purity of daptomycin in the samples was calculated as the ratio of the peak area of the intact daptomycin peak at 215 nm relative to the area sum of all daptomycin-related peaks identified at 215 nm. It was found that within the first 7 days of incubation under physiological conditions, the purity of the daptomycin, which was continuously released from transient daptomycin-linker hydrogel conjugate was constantly at around 85%, whereas the purity of the free daptomycin in the solution control sample dropped to 72% at day seven.

### Example 9

### Degradation Study of a Transient Daptomycin-Linker Hydrogel Conjugate

The transient daptomycin-linker hydrogel conjugate was analyzed regarding carrier degradation. For that purpose, the transient daptomycin-linker PEG-conjugate 6b was incubated at pH 7.4 and 37° C. The sample was visually checked for the presence of the solid carrier particles on a daily basis. As soon as no particles could be detected in the sample anymore, the material was deemed to be fully degraded to soluble products. It was found that the transient daptomycin-linker PEG-hydrogel conjugate 6b was fully degraded after about 40 days.

### Example 10

### Quantification of Daptomycin Concentrations in Rabbit Plasma

Daptomycin concentrations in rabbit plasma were determined after plasma protein precipitation via liquid chromatography separation and detection by LC-MS. As internal standard deuterated daptomycin-D5 peptide was used. LC-MS analysis was carried out by using a UHPLC system coupled to a triple quadrupole mass spectrometer via an ESI probe. Chromatography was performed on a C18 analytical UHPLC column. UPLC grade water containing 0.1% formic acid (v/v) was used as mobile phase A and UPLC grade acetonitrile with 0.1% formic acid as mobile phase B. The gradient system comprised a linear increase from 20% B to 45% B in 10 min. Mass analysis was performed in MRM mode with the selected transitions for daptomycin and the internal standard daptomycin-D5.

Calibration standards of daptomycin in blank plasma were prepared as follows: thawed K₂-EDTA rabbit plasma was homogenized. The daptomycin formulation was spiked into blank plasma at concentrations between 1000 ng/mL and 2 ng/mL. These solutions were used for the generation of a calibration curve. Calibration curves were weighted 1/×².

For sample preparation, 70 μL of sample were spiked with 20 μL of internal standard solution. Subsequently, the mixture was spiked with 40 μL of 0.5 M citrate buffer pH 4.0 and incubated for 30 min at room temperature. Protein precipitation was carried out by addition of 270 μL of room temperature methanol. 200 μL of the supernatant were transferred into a new well-plate and evaporated to dryness (under a gentle nitrogen stream at 45° C.). 50 μL of reconstitution solvent (H₂O/MeOH 1:1+1.0% FA) were used to dissolve the residue by intensive shaking. 10 μL were injected into the LC-MS system.

### Example 11

Pharmacokinetic Profiles of Daptomycin in New Zealand White Rabbits after Intraarticular (IA) Injections with a Transient Daptomycin-Linker Hydrogel Conjugate

This study was performed in order to investigate the systemic pharmacokinetics of daptomycin in male New Zealand White (NZW) rabbits following intraarticular administration of transient daptomycin-linker PEG-hydrogel conjugate 6b. Animals (n=9 per group) received a single IA injection of 300 μL transient daptomycin-linker PEG-hydrogel conjugate 6b formulation (15 mg daptomycin nominal) in the right knee and 300 μL vehicle in the left knee. Three animals from each group were sacrificed three days, two weeks, and six weeks after dosing. Blood samples for PK analysis were collected and processed to plasma at predose and 0.5, 1, 2, 4, 8, 12, 24, 48, 72, 96, 120, 144, 168, 336 hours post dose (PK blood samples were only collected until 72 hours post dose from animals with three days inlife). Moreover, blood was collected for clinical chemistry and hematology at predose, day three, day seven*, week two*, and week six* (*in the appropriate groups). Visual inspection and palpation (such as reddening/swelling) were performed in the first seven days after injection. Hereafter, visual inspection and palpation was done once a week. Upon sacrifice all knees were sampled for histopathological examination.

Results: Dose administrations were well tolerated with no visible signs of discomfort during administration and following administration. No dose site reactions were observed any time throughout the study and all animals showed normal behavior and no knee swelling or warming. After intraarticular injection of the transient daptomycin-linker PEG-hydrogel conjugate 6b, sustained PK plasma concentrations above 100 ng/mL were detected over the time course of one week after injection.

### Abbreviations

ACN Acetonitrile
AcOH Acetic Acid
Asp Aspartic Acid
Bn Benzyl
Crl Charles River Laboratories
DBU 1,8-Diazabicyclo[5.4.0]undec-7-ene
DCC Dicyclohexylcarbodiimide
DCM Dichloromethane
DIPEA N, A-Diisopropylethylamine
DMAP 4-(Dimethylamino)pyridine
DMSO Dimethyl Sulfoxide
DPHS Dipolyhydroxystearate
EDC N-(3-Dimcthylaminopropyl)-N′-ethylcarbodiimide Hydrochloride
EDTA Ethylenediaminetetraacetic Acid
eqv. Equivalents
ESI Electrospray Ionization
EtOH Ethanol
FA Formic Acid
Fmoc Fluorenylmethyloxycarbonyl
HFIP 1,1,1,3,3,3-Hexafluoro-2-propanol
HOBt 1-Flydroxybenzotriazole
FIPLC High-Performance Liquid Chromatography
IA Intraarticular
LC-MS Mass Spectrometry Coupled Liquid Chromatography
LPLC Low Pressure Liquid Chromatography
MeCN Acetonitrile
MeOH Methanol
MES 2-(N-Morpholino)ethanesulfonic acid
MRM Multiple Reaction Monitoring
MTBE tert-Butyl Methyl Ether
Mw Molecular Weight
NHS A-Hydroxysuccinimide
NMP M- Methyl-2-pyrrolidone
NZW New Zealand White Rabbits
OD600 Optical Density Measured at 600 nm Wavelength
OPA o-Phthalaldehyde
OxymaPure® Ethyl cyano(hydroxyimino)acetate
PE Polyethylene
PEG Poly(ethylene glycol)
PK Pharmacokinetic/s
PTFE Polytetrafluoroethylene
PyBOP Benzotriazol-1-yl-oxytripyrrolidinophosphonium Hexafluorophosphate
QAAA Quantitative Amino Acid Analysis
RP-HPLC Reversed Phase High-Performance Liquid Chromatography
RP-LPLC Reversed Phase Low Pressure Liquid Chromatography
r.t. Room Temperature
SEC Size-exclusion chromatography
TES Triethylsilane
TFA Trifluoroacetic Acid
THF Tetrahydro furane
TMEDA N, N, N′,N′-Tetramethylethylenediamine
Tmob 2,4,6-Trimethoxybenzyl
Trt Trityl
TSTU N,N,N′,N′-Tetramethyl-O—(N-succinimidyl)uronium Tetrafluorborate
Tween 20 Polyethylene Glycol Sorbitan Monolaurate
UHPLC Ultra High Performance Liquid Chromatography
UPLC Ultra Performance Liquid Chromatography
UPLC-MS Mass Spectrometry Coupled Ultra Performance Liquid Chromatography

1. A conjugate comprising a water-insoluble hydrogel Z, wherein said conjugate comprises a plurality of moieties -L²-L¹-D covalently conjugated to Z,
wherein
each -D is drug moiety;
each -L¹- is independently a linker moiety to which -D is covalently and reversibly conjugated;
each -L²- is independently either a chemical bond or a spacer moiety;
Z is a PEG-based hydrogel comprising a plurality of backbone moieties that are crosslinked via crosslinker moieties —CL-, either directly or via a spacer moiety —SP— between a crosslinker moiety and —CL-, and wherein —CL- is of formula (A) 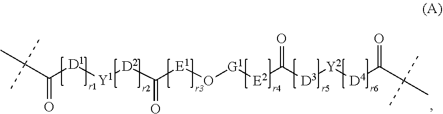
wherein
dashed lines indicate attachment to a backbone moiety or to a spacer moiety —SP—;
—Y¹— is of formula 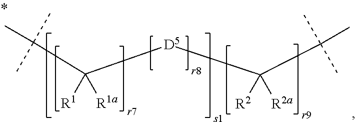
wherein the dashed line marked with the asterisk indicates attachment to -D¹- and the unmarked dashed line indicates attachment to -D²-;
—Y²— is of formula 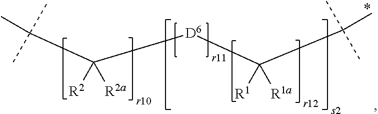
wherein the dashed line marked with the asterisk indicates attachment to -D⁴- and the unmarked dashed line indicates attachment to -D³-;
-E¹- is of formula 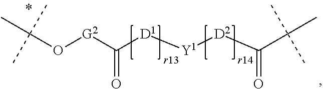
wherein the dashed line marked with the asterisk indicates attachment to —(C═O)— and the unmarked dashed line indicates attachment to —O—;
-E²- is of formula 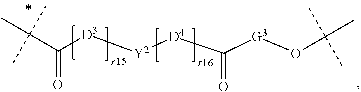
wherein the dashed line marked with the asterisk indicates attachment to -G¹- and the unmarked dashed line indicates attachment to —(C═O)—;
-G¹- is of formula 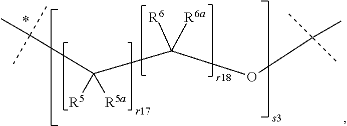
wherein the dashed line marked with the asterisk indicates attachment to —O— and the unmarked dashed line indicates attachment to -E²-;
-G²- is of formula 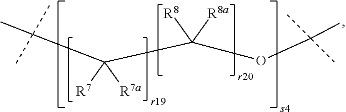
wherein the dashed line marked with the asterisk indicates attachment to —O— and the unmarked dashed line indicates attachment to —(C═O)—;
-G³- is of formula 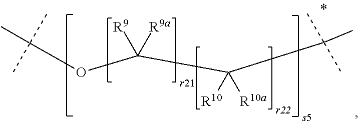
wherein the dashed line marked with the asterisk indicates attachment to —O— and the unmarked dashed line indicates attachment to —(C═O)—;
-D¹-, -D²-, -D³-, -D⁴-, -D⁵- and -D⁶- are identical or different and each is independently of the others selected from the group comprising —O—, —NR¹¹—, —N⁺R¹²R^{12a}—, —S—, —(S═O)—, —(S(O)₂)—, —C(O)—, —P(Q)R¹³—, —P(O)(OR¹³) and —CR¹⁴R^{14a}—;
—R¹, —R^{1a}, —R², —R^{2a}, —R³, —R^{3a}, —R⁴, —R^{4a}, —R⁵, —R^{5a}, —R⁶, —R^{6a}, —R⁷, —R^{7a}, —R⁸, —R^{8a}, —R⁹,
—R^{9a}, —R¹⁰, —R^{10a}, —R¹¹, —R¹², —R^{12a}, —R¹³, —R¹⁴ and —R^{14a} are identical or different and each is independently of the others selected from the group consisting of —H and C₁₋₆ alkyl;
optionally, one or more of the pairs —R¹/—R^{1a}, —R²/—R^{2a}, —R³/—R^{3a}, —R⁴/—R^{4a}, —R¹/—R², —R³/—R⁴, —R^{1a}/—R^{2a}, —R^{3a}/—R^{4a}, —R¹²/—R^{12a}, and —R¹⁴/—R^{14a} form a chemical bond or are joined together with the atom to which they are attached to form a C₃₋₈ cycloalkyl or to form a ring A or are joined together with the atom to which they are attached to form a 4- to 7-membered heterocyclyl or 8- to 11-membered heterobicyclyl or adamantyl;
A is selected from the group consisting of phenyl, naphthyl, indenyl, indanyl and tetralinyl;
r1, r2, r5, r6, r13, r14, r15 and r16 are independently 0 or 1;
r3, r4 are independently 0, 1, 2, 3, or 4, with the provision that r3+r4≥1;
r7, r8, r9, r10, r11, r12 are independently 0, 1, 2, 3, or 4;
r17, r18, r19, r20, r21 and r22 are independently 1, 2, 3, 4, 5, 6, 7, 8, 9 or 10;
s1, s2, s4, s5 are independently 1, 2, 3, 4, 5 or 6; and
s3 ranges from 1 to 900.

2. The conjugate of claim 1, wherein r3 and r4 are both 1.

3. The conjugate of claim 1 or 2, wherein r1, r2, r5 and r6 are 0.

4. The conjugate of any one of claims 1 to 3, wherein s3 ranges from 15 to 100.

5. The conjugate of any one of claims 1 to 4, wherein s3 ranges from 20 to 50.

6. The conjugate of any one of claims 1 to 5, wherein a moiety —CL- has a molecular weight ranging from 0.2 kDa to 25 kDa

7. The conjugate of any one of claims 1 to 6, wherein a moiety —CL- is selected from the group consisting of 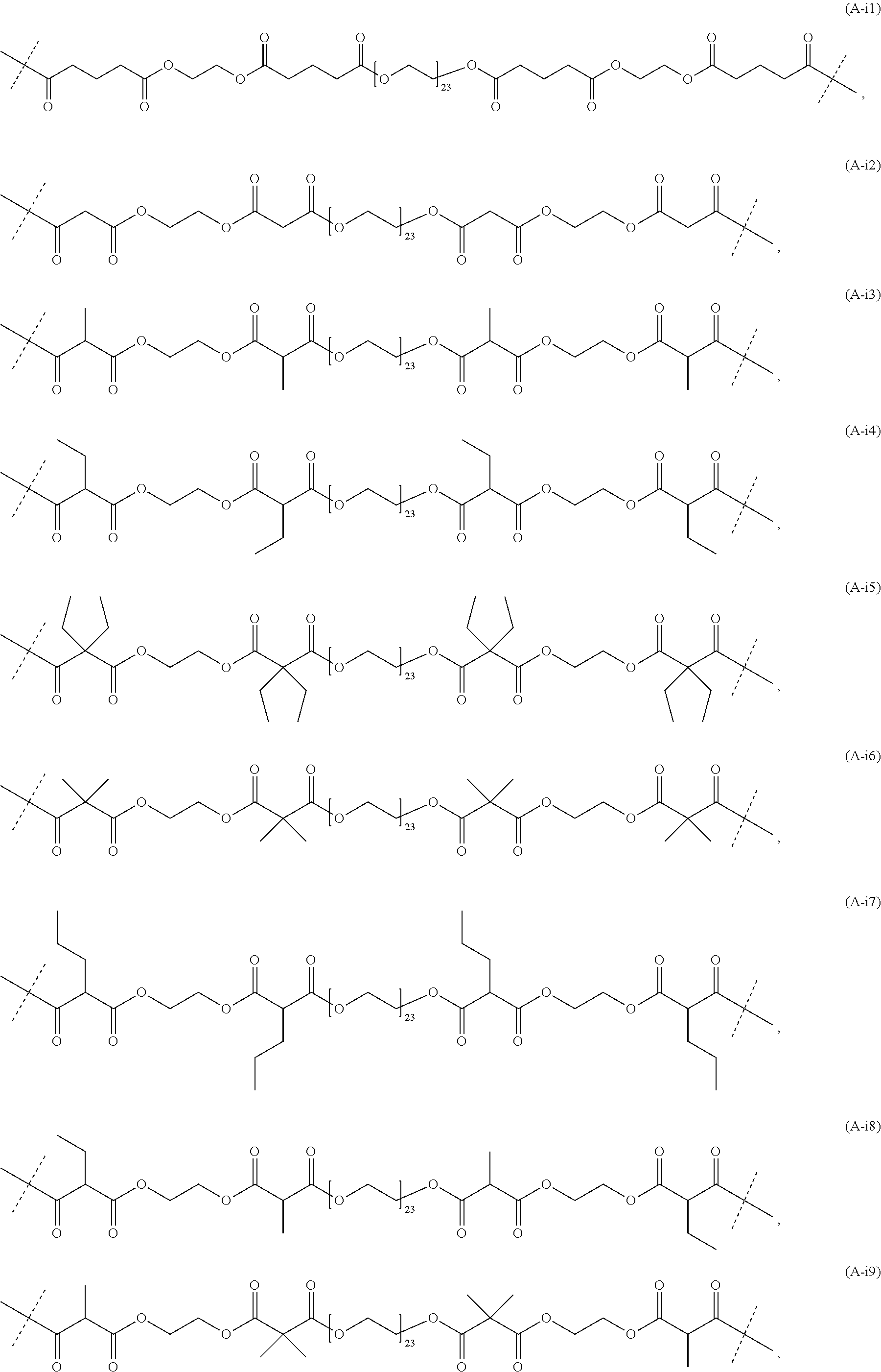 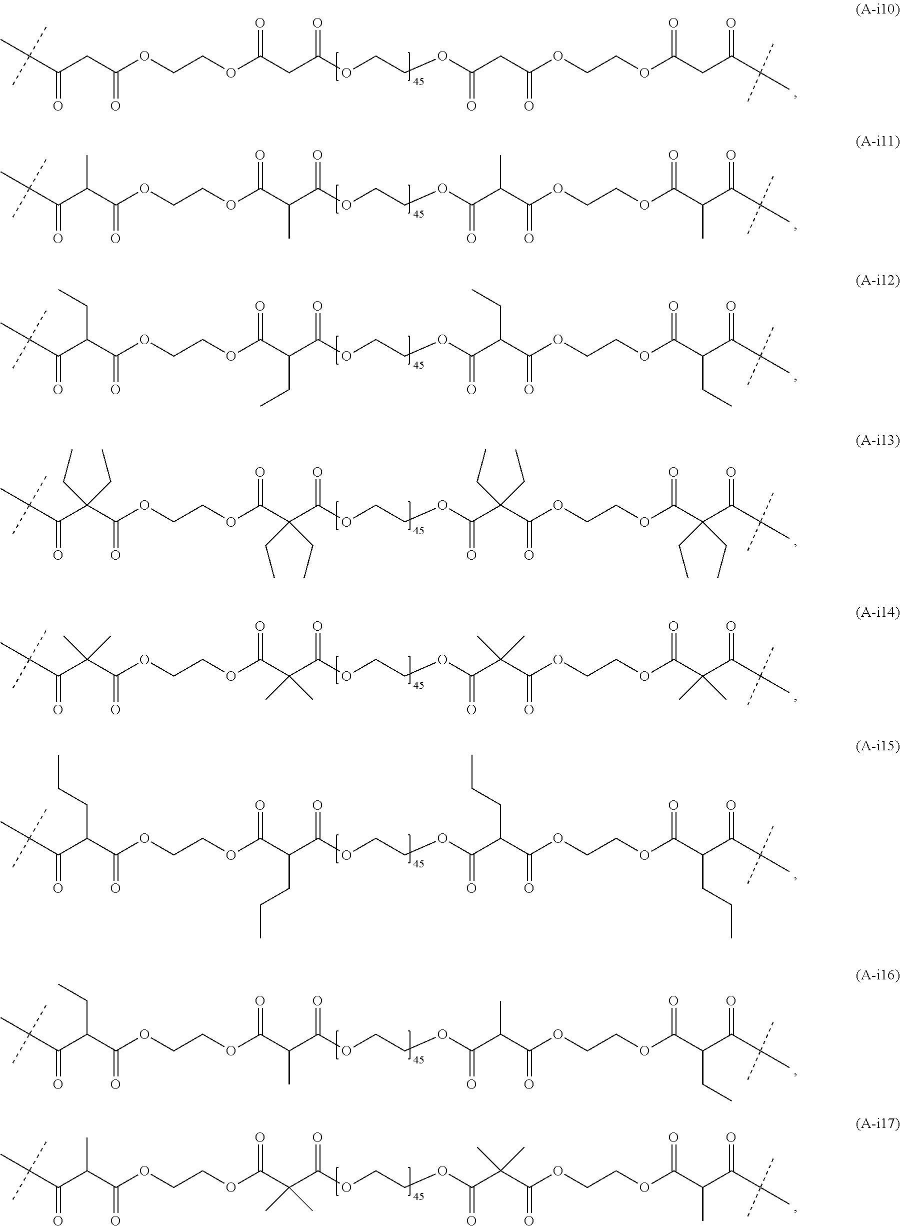 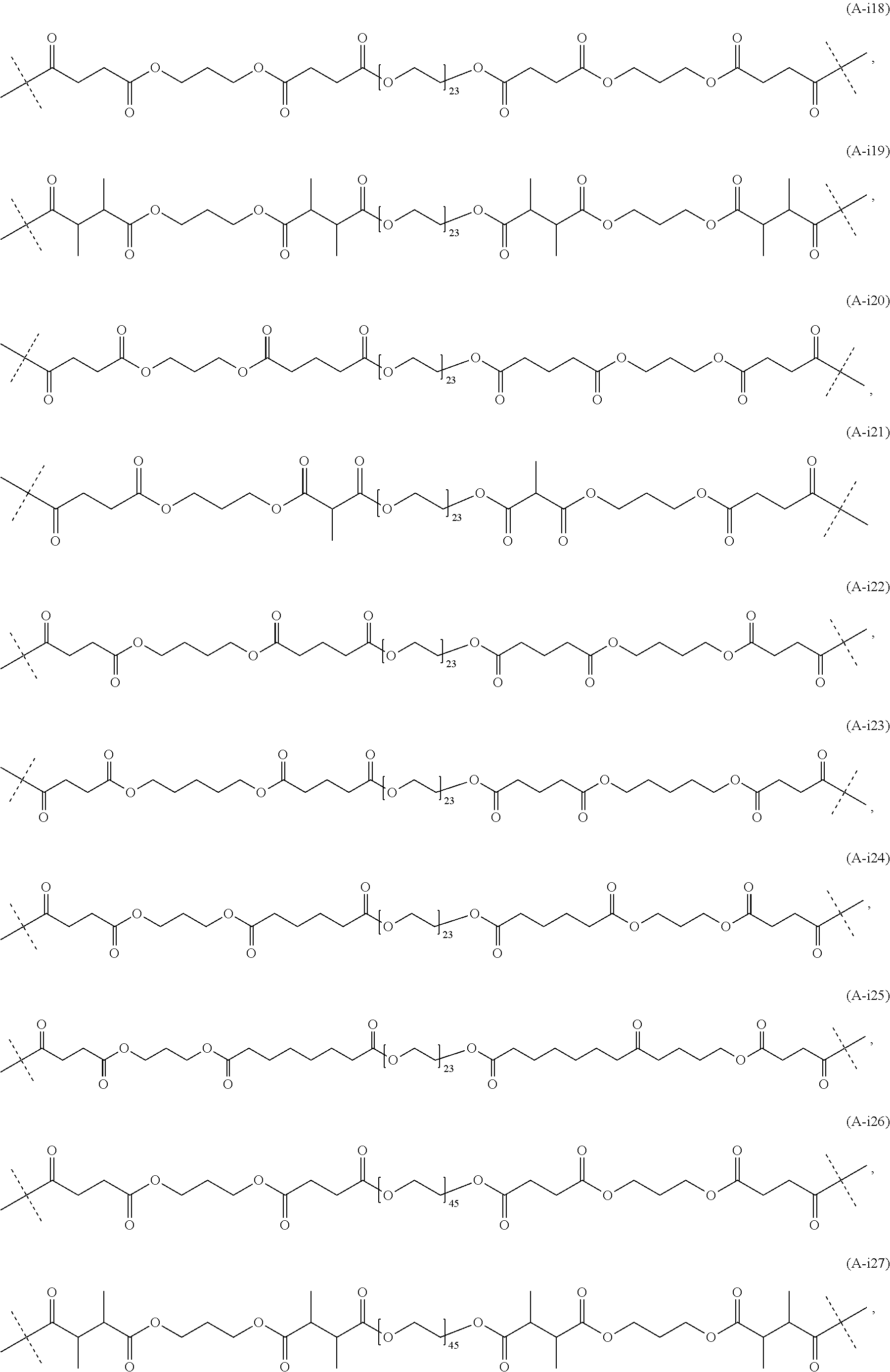 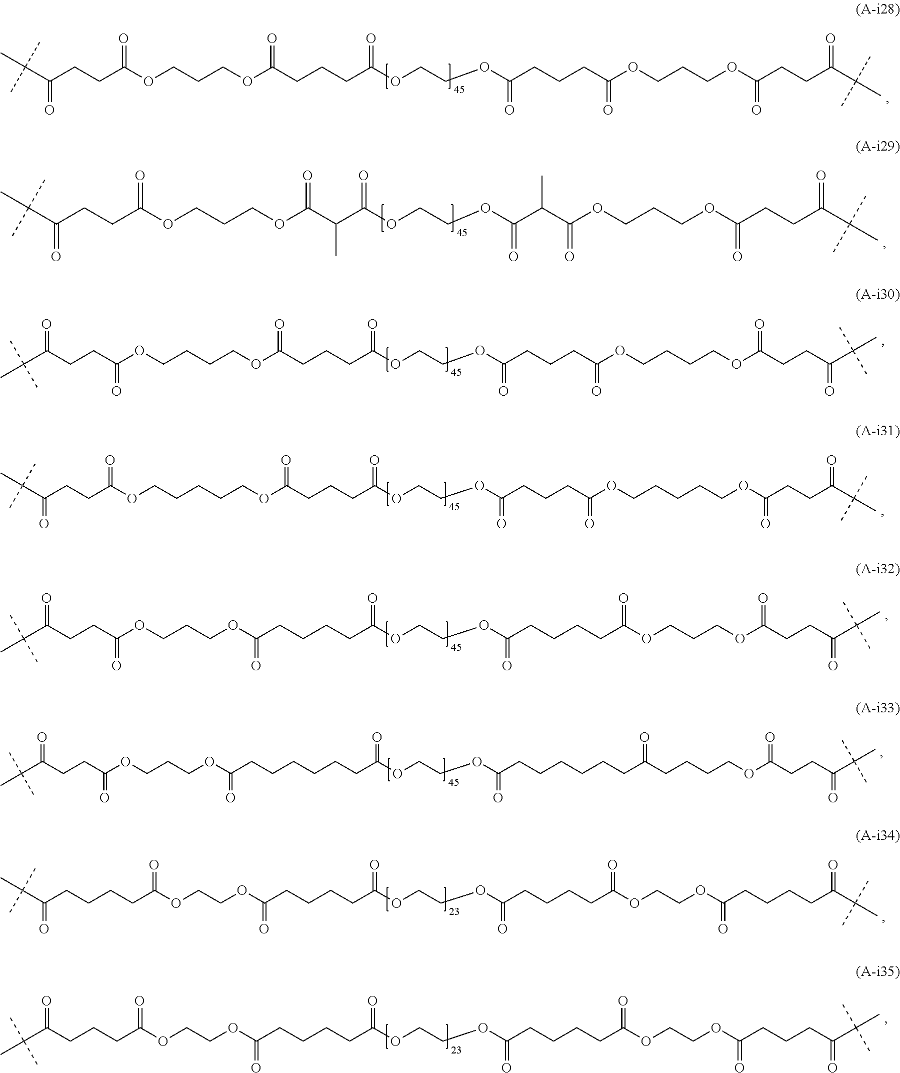 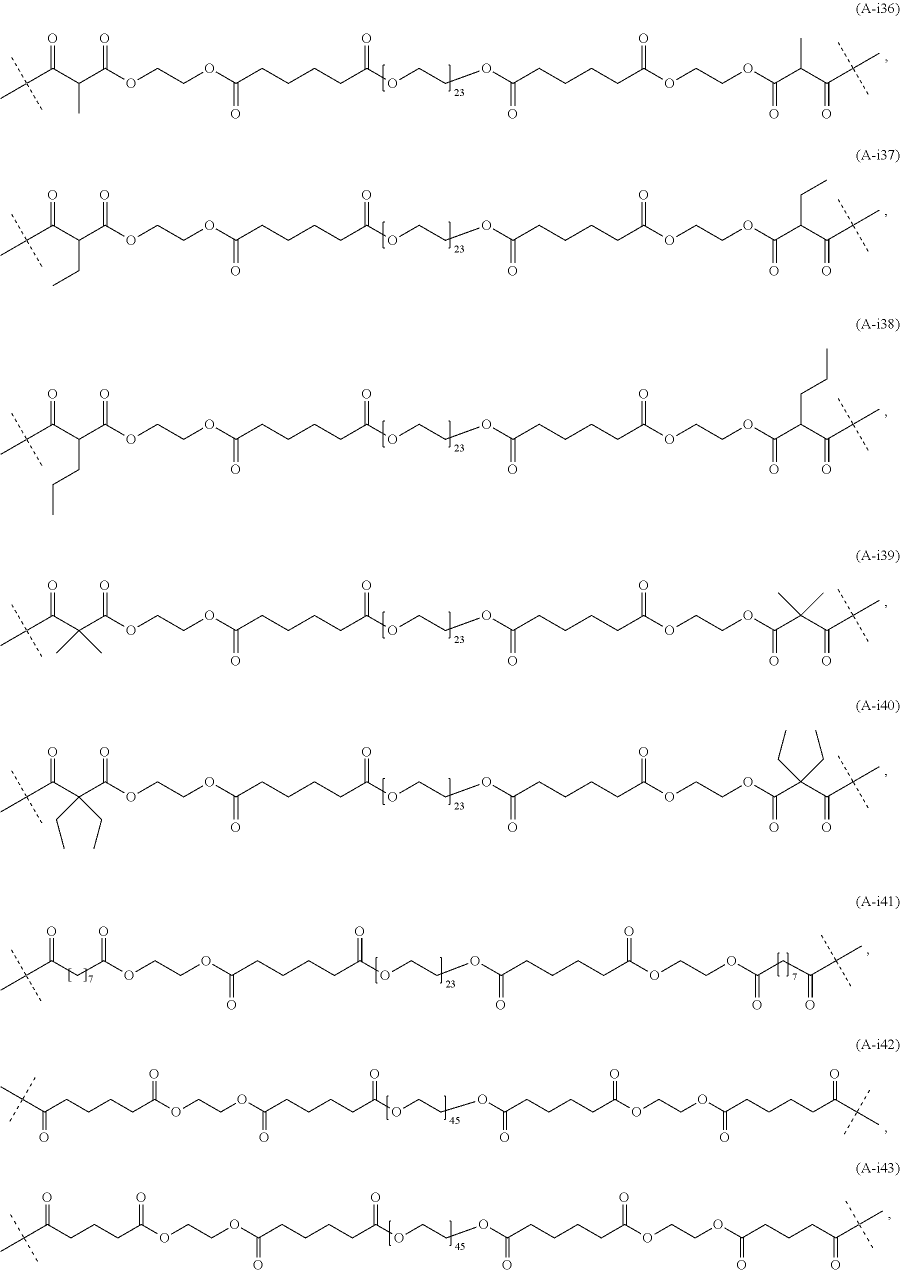 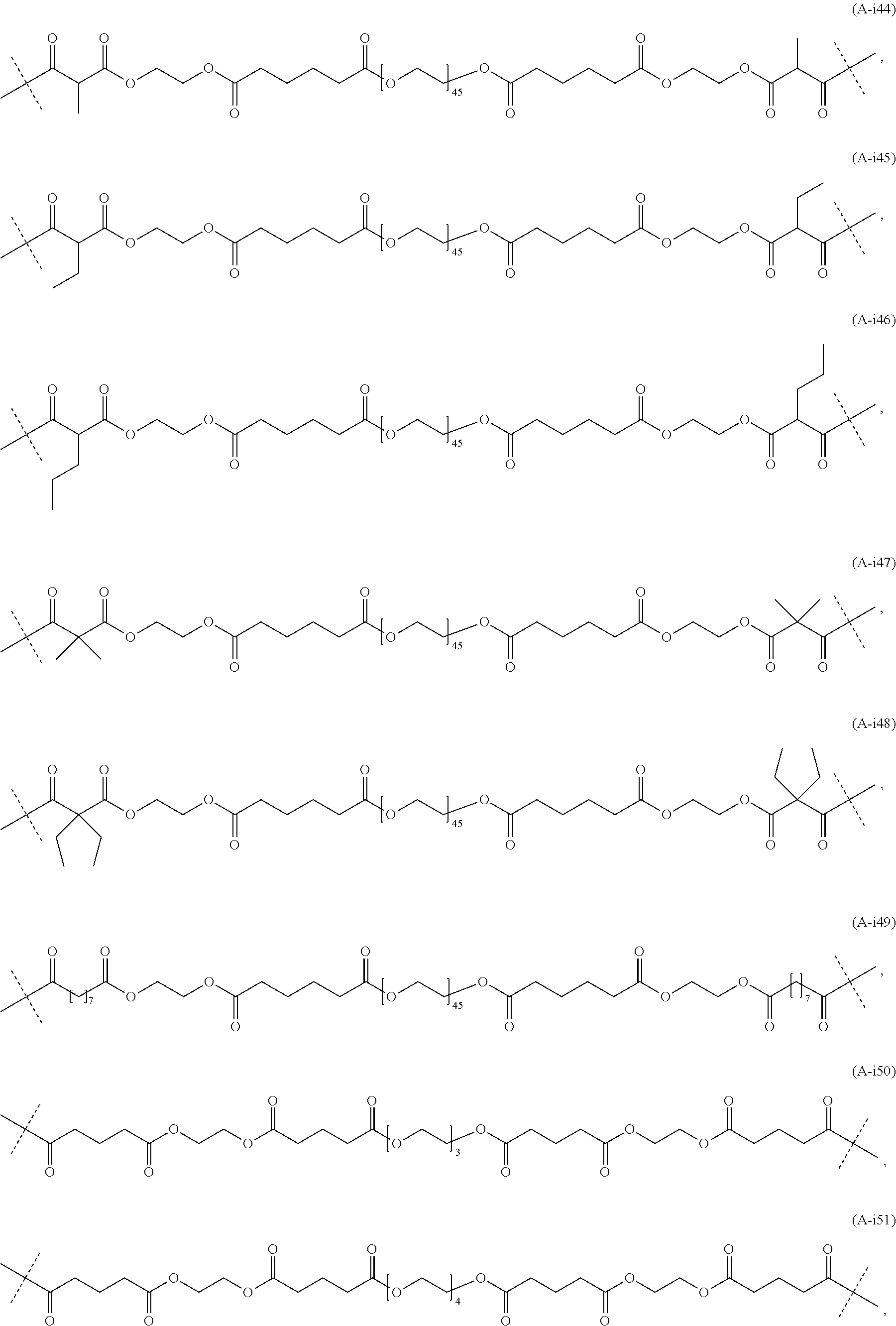 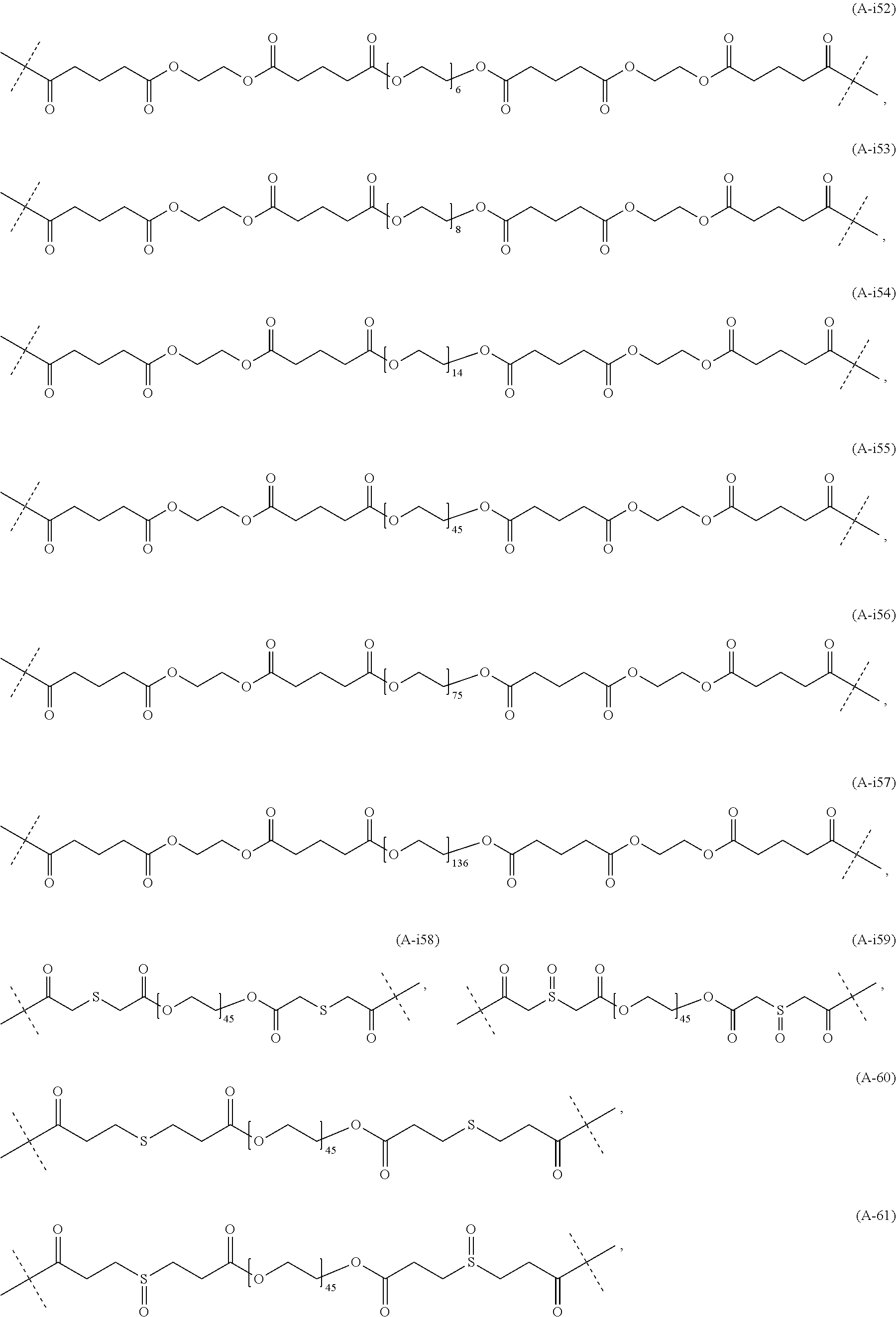 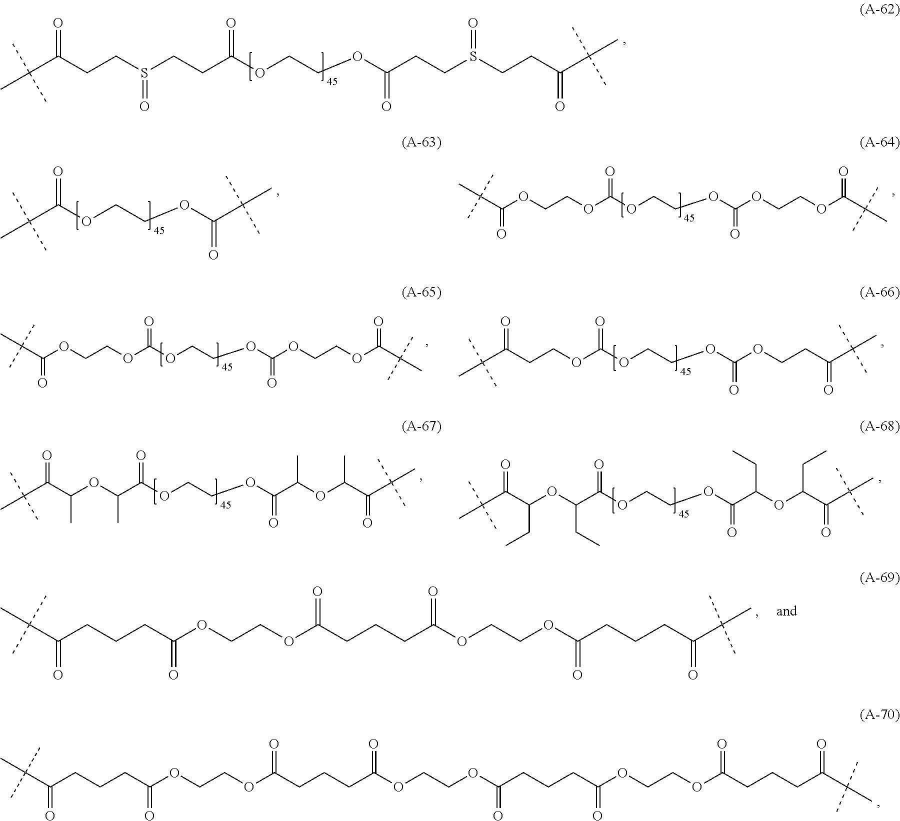
wherein
dashed lines indicate attachment to a backbone moiety or to a spacer moiety —SP—.

8. The conjugate of any any one of claims 1 to 7, wherein a backbone moiety has a molecular weight ranging from 1 kDa to 20 kDa.

9. The conjugate of any one of claims 1 to 8, wherein -L¹- is of formula (I): 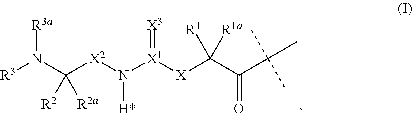
wherein the dashed line indicates the attachment to a nitrogen, hydroxyl or thiol of -D;
—X— is selected from the group consisting of —C(R⁴R^{4a})—, —N(R⁴)—, —O—, —C(R⁴R^{4a})—C(R⁵R^{5a})—, —C(R⁵R^{5a})—C(R⁴R^{4a})—, —C(R⁴R^{4a})—N(R⁶)—, —N(R⁶)—C(R⁴R^{4a})—, —C(R⁴R^{4a})—O—, —O—C(R⁴R^{4a})—, and —C(R⁷R^{7a})—,
X¹ is selected from the group consisting of C and S(O);
—X²— is selected from the group consisting of —C(R⁸R^{8a})— and —C(R⁸R^{8a})—C(R⁹R^{9a})—;
═X is selected from the group consisting of ═O, ═S, and ═N—CN;
—R¹, —R^{1a}, —R², —R^{2a}, —R⁴, —R^{4a}, —R⁵, —R^{5a}, —R⁶, —R⁸, —R^{8a}, —R⁹ and —R^{9a} are independently selected from the group consisting of —H and C₁₋₆ alkyl;
—R³ and —R^{3a} are independently selected from the group consisting of —H and C₁₋₆ alkyl, provided that in case one of —R³ and —R^{3a} or both are other than —H they are connected to N to which they are attached through an sp³-hybridized carbon atom;
—R⁷ is selected from the group consisting of —N(R¹⁰R^{10a}) and —NR¹⁰—(C═O)—R¹¹;
—R^{7a}, —R¹⁰, —R^{10a} and —R¹¹ are independently selected from the group consisting of —H and C₁₋₆ alkyl;
alternatively, one or more of the pairs —R^{1a}/—R^{4a}, —R^{1a}/—R^{5a}, —R^{1a}/—R^{7a}, —R^{4a}/—R^{5a} and —R^{8a}/—R^{9a} form a chemical bond;
alternatively, one or more of the pairs —R¹/—R^{1a}, —R²/—R^{2a}, —R⁴/—R^{4a}, —R⁵/—R^{5a}, —R⁸/—R^{8a} and —R⁹/—R^{9a} are joined together with the atom to which they are attached to form a C₃₋₁₀ cycloalkyl or 3- to 10-membered heterocyclyl;
alternatively, one or more of the pairs —R¹/—R⁴, —R¹/—R⁵, —R¹/—R⁶, —R¹/—R^{7a}, —R⁴/—R⁵, —R⁴/—R⁶, —R⁸/—R⁹ and —R²/—R³ are joined together with the atoms to which they are attached to form a ring A;
alternatively, R³/R^{3a} are joined together with the nitrogen atom to which they are attached to form a 3- to 10-membered heterocycle;
A is selected from the group consisting of phenyl; naphthyl; indenyl; indanyl; tetralinyl; C₃₋₁₀ cycloalkyl; 3- to 10-membered heterocyclyl; and 8- to 11-membered heterobicyclyl; and
wherein -L¹- is substituted with -L²- and wherein -L¹- is optionally further substituted, provided that the hydrogen marked with the asterisk in formula (II) is not replaced by -L²- or a substituent.

10. The conjugate of any one of claims 1 to 9, wherein -L²- is a spacer moiety.

11. The conjugate of any one of claims 1 to 10, wherein -D is an antibiotic moiety.

12. The conjugate of any one of claims 1 to 11, wherein -D is selected from the group consisting of aminoglycosides, tetracycline antibiotics, amphenicols, pleuromutilins, macrolid antibiotics, lincosamides, steroid antibiotics, antifolate antibiotics, sulfonamides, topoisomerase inhibitors, quinolones, fluoroquinolones, nitroimidazole antibiotics, nitrofuran antibiotics, rifamycins, glycopeptides, penicillins, cephalosporins, monobactams, beta-lactamase inhibitors, polymyxin antibiotics, lipopeptide antibiotics, oxazolidinon, antimicrobial peptides, antimicrobial proteins, porphyrins, azole antifungals, polyenes, antiprotozoal drugs, fosfomycin, cycloserine, and bacitracin.

13. The conjugate of any one of claims 1 to 12, wherein -D is daptomycin.

14. A pharmaceutical composition comprising the conjugate of any one of claims 1 to 13 and at least one excipient.

15. The conjugate of any one of claims 1 to 13 or the pharmaceutical composition of claim 14 for use as a medicament.

16. The conjugate of any one of claims 11 to 13 or the pharmaceutical composition of claim 14 for use in the in the diagnosis, prophylaxis or treatment of a disease that can be treated with the conjugates of the present invention.

17. The conjugate of any one of claims 11 to 13 or the pharmaceutical composition of claim 14 for use as an antibiotic.

18. The conjugate of any one of claims 11 to 13 or the pharmaceutical composition of claim 14 for use in a method of preventing or treating a joint infection.
